(19) United States
(12) Patent Application Publication
(10) Pub. No.: US 2010/0069927 A1
(43) Pub. Date: Mar. 18, 2010

(54) Polymeric Masking Materials for Spanning Wound Sites, and Methods of Use Thereof

(75) Inventors: Jeffrey G. Clark, Raleigh, NC (US); Robert Naslund, Raleigh, NC (US); Kenneth E. Stockman, Raleigh, NC (US)

Correspondence Address: FOLEY HOAG, LLP;PATENT GROUP, WORLD TRADE CENTER WEST, 155 SEAPORT BLVD, BOSTON, MA, 02110, US

(73) Assignee: HyperBranch Medical Technology, Inc., Durham, NC (US)

(21) Appl. No.: 12/177,512

(22) Filed: Jul. 22, 2008

Related U.S. Application Data
(60) Provisional application No. 60/951,283, filed on Jul. 23, 2007.

(51) Int. Cl. A61B 17/03

(52) U.S. Cl. 606/151; 606/214

(57) ABSTRACT

One aspect of the present invention generally relates to methods of sealing a defect, wound or void using a two component system composed of masking material, which creates an anti-adhesion barrier, and a covering material. In certain embodiment, the covering material comprises an in situ polymerizing sealant. In certain embodiments, the in situ polymerizing sealant is a hydrogel which binds to the healthy tissue but remain unadhered to the area under the masking material. In certain embodiments, the masking material is also a hydrogel. In certain embodiments, normal biological processes may dissolve away the masking material leaving a protective cover of polymerized sealant over the wound site.

### RELATED APPLICATIONS

This application claims the benefit of priority to U.S. Provisional Patent Application Ser. No. 60/951,283, filed Jul. 23, 2007.

### BACKGROUND

Bandages and bandaging methods play an important role in helping patients recover from surgery or trauma. There exists a need for bandages which are useful in treating patients suffering from a variety of internal and topical conditions, including lacerations, tears, wounds, ulcers, anastamoses, and surgical procedures. Bandages which can generally be used in any indication or application for which a suture or staple is presently used often will provide a better outcome than a suture or staple. Bandages can also be applied more quickly to the injury site and often provide a better seal over the wound and healing. Various medicinal applications for bandages and bandaging methods are described below.

### Skin Lacerations

Skin lacerations are tears in the skin produced by accidents, trauma, or as a result of a surgical procedure. Lacerations often require treatment in order to close the hole in the skin, stop bleeding, and prevent infection. Minor lacerations in the skin may be treated using an adhesive tissue to cover the wound. However, larger lacerations often require sutures or a glue to help seal the wound. For example, it is generally recommended that sutures or a glue be used to treat lacerations deeper than 0.25 inches having a jagged edge or loose flap of tissue. The location of the laceration may also affect the form of treatment. For example, it is advantageous to treat a skin laceration on a joint using a glue because adhesive tissue tends to limit mobility of the joint. The use of sutures or glues to treat skin lacerations can also reduce the chance of scar formation. The bandages and bandaging methods of the invention may be used to treat skin lacerations.

### Liver Lacerations

Lacerations of the liver can occur from trauma or as a result of a surgical procedure. The liver is a highly vascularized organ and bleeds profusely when lacerated or traumatized. Liver lacerations are difficult to repair owing to the nature of liver tissue. Liver tissue has very weak cohesive strength, and, consequently, sutures and staples are not satisfactory because they may pull through the liver tissue. The lack of satisfactory wound treatment methods for liver lacerations combined with the fact that it is difficult to reach the veins that feed the liver renders liver lacerations particularly serious. In fact, severe lacerations of the liver often result in the patient's death due to bleeding. Thus, new bandages and bandaging methods to treat liver lacerations are needed.

### Lung Surgery

The bandages and bandaging methods of the present invention are useful in lung surgery. Types of lung surgery include lobectomy, lung biopsy, lung-tissue removal, pneumonectomy, thoracoscopy, and thoracotomy. Risks associated with lung surgery include wound infection; post-surgical internal bleeding; air leaks through the lung wall; pain or numbness at the incision site; and inflammation of the lungs (pneumonia). Further, air leakage is frequently observed after thoracic procedures, such as pulmonary resection and decortication. It is important to create an air-tight seal so as to prevent or reduce severe complications, such as bronchopleural fistulas and infection resulting from extended chest tube drainage, extended recovery time, and postoperative morbidity related to pulmonary surgery. The bandages and bandaging methods of the invention should decrease or eliminate some of the problematic aspects of lung surgery, such as treatment of pneumothorax and pulmonary leaks.

### Cornea—Corneal Lacerations/Perforations

Corneal perforations are produced by a variety of medical conditions (e.g., infection, inflammation, xerosis, neurotrophication, and degeneration) and traumas (chemical, thermal, surgical, and penetrating). Unfortunately, corneal perforations often lead to loss of vision and a decrease in an individual's quality of life. Depending on the type and the origin of the perforation, different treatments may be effective, ranging from suturing the wound to a cornea graft. However, the surgical procedures are difficult given the delicate composition of the cornea and the severity of the wound which increase the likelihood for leakage and severe astigmatism after surgery. In certain cases, for example, perforations that cannot be treated by standard suture procedures, tissue adhesives (glues) are used to repair the wound. This type of treatment is very attractive because the method is simple, quick and safe, and corresponds to the requirement of a quick restoration of the integrity of the globe, avoiding further complications. Besides an easy and fast application on the wound, the characteristics of an adhesive include: 1) bind to the tissue (necrosed or not, very often wet) with an adequate adhesion force; 2) be non-toxic; 3) be biodegradable or resorbable; 4) be sterilizable; and 5) not interfere with the healing process. The bandages and bandaging methods may be helpful in such an application.

Various alkyl-cyanoacrylates are available for the repair of small perforations. However, these “super glues” present major inconveniences. Their monomers, in particular those with short alkyl chains, can be toxic, in part due to their ability to produce formaldehyde in situ. They also polymerize too quickly leading to applications that might be difficult and, once polymerized, the surface of the glue is rough and hard which leads to patient discomfort and a need to wear contact lens. Even though cyanoacrylate is tolerated as a corneal sealant, a number of complications have been reported including cataract formation, corneal infiltration, glaucoma, giant papillary conjunctivitis, and symblepharon formation. Furthermore, in more than 60% of the patients, additional surgical intervention is needed.

Other glues have also been developed. Adhesive hemostats, based on fibrin, are usually constituted of fibrinogen, thrombin and factor XIII. Systems with fibrinogen and photosensitizers activated with light are also being tested. If adhesive hemostats have intrinsic properties which meet the requirements for a tissue adhesive, then autologous products (time consuming in an emergency) or severe treatments before clinical use are needed to avoid any contamination to the patient. An ideal sealant for corneal perforations should 1) not impair normal vision, 2) quickly restore the intraocular pressure (IOP), 3) maintain the structural integrity of the eye, 4) promote healing, 5) adhere to moist tissue surfaces, 6) possess solute diffusion properties which are molecular weight dependent and favorable for normal cornea function, 7) possess rheological properties that allow for controlled placement of the polymer on the wound, and 8) polymerize under mild conditions.

The use of sutures has limitations and drawbacks. First, suture placement itself inflicts trauma to corneal tissues, especially when multiple passes are needed. Secondly, although suture material has improved, sutures such as 10-0 nylon (which is the suture of choice in the cornea and elsewhere) can act as a nidus for infection and incite corneal inflammation and vascularization. With persistent inflammation and vascularization, the propensity for corneal scarring increases. Thirdly, corneal suturing often yields uneven healing and resultant regular and irregular astigmatism. Postoperatively, sutures are also prone to becoming loose and/or broken and require additional attention for prompt removal. Finally, effective suturing necessitates an acquired technical skill that can vary widely from surgeon to surgeon and can also involve prolonged operative time.

### Oculoplastics—Blepharoplasty Incisions

Blepharoplasty is an operation to remove excess skin, fat and muscle from around the eyes to correct droopy eyelids and bagginess under the eyes. It can be performed on the upper lids and lower lids, at the same time or separately. The operation may be done using either conventional or laser techniques. For surgery on the upper eyelids, cuts are made into the natural lines and creases in the lid, and into the laughter lines at the corner of the eye. For surgery on the lower eyelids, a cut is usually made just below the eyelashes. This means the scars run along the eye's natural folds, concealing them as much as possible. Excess fat, muscle and loose skin are removed, and the cut is closed using sutures. If only fat is being removed, sometimes the cut is made on the inside of the lower eyelid, leaving no visible scar. The bandaging methods of the present invention may provide a more effective means to secure the cuts made during surgery.

### Gastrointestinal Anastomosis

The bandaging methods of the present invention should also be useful in gastrointestinal anastomosis procedures. Gastrointestinal anastomosis is the technique of joining two pieces of bowel together. There are many techniques for gastro-intestinal anastomosis, including both mechanical stapled techniques and hand-sutured procedures. The technique may involve a simple end-end anastomosis of two pieces of jejunum, a more complex colo-anal anastomosis, or a biliary enteric join. One problem with techniques employing sutures or staples is that leakage may occur around the sutures or staples. See, for example, Bruce et al. Br. J. Surg. 88:1157-1168 (2001) reporting leakage rates of 5-8%. However, sealants and methods of the invention could be used to supplement the sutures or staples used in intestinal anastomoses, providing a better seal that reduces leakage. Compositions and procedures for proper sealing the consequences of a failed anastomosis are severe and frequently life-threatening. Although failures can be caused by myriad factors, including poor surgical technique (e.g., sutures that were not inserted correctly; knots that were tied too tightly rendering the ends ischaemic; or incorrect use of a staple gun), the sealants and methods of the invention should decrease or eliminate some of the causes of failed gastrointestinal anastomosis procedures.

### Prostatectomy Urethral-Bladder Anastomosis

The bandages and bandaging methods of the present invention should be useful in prostatectomy urethral-bladder anastomosis procedures. Prostatectomy urethral-bladder anastomosis is the technique of joining together a patient's ureter and bladder after surgical removal of his prostate gland. Failures are caused by myriad factors, including poor surgical technique (e.g., sutures that were not inserted correctly; knots that were tied too tightly rendering the ends ischaemic). The sealants and methods of the invention should decrease or eliminate some of the causes of failed prostatectomy urethral-bladder anastomosis procedures.

### Tissue Plane Applications

The bandages and bandaging methods of the invention can be applied to two planes of tissue and then these two tissues can be sealed together. Over time the bandage degrades as new tissue grows into the area. Applications include a number of cosmetic and tissue restoration surgeries. The sealant is used when the procedures involve significant tissue plane separation that may result in formation of seroma with associated complications, such as infection, e.g., general surgery procedures, such as mastectomies and lumpectomies, and plastic surgery procedures, such as abdominoplastys, rhytidectomy or rhinoplastys, mammaplasty and reconstruction, forehead lifts and buttocks lifts, as well as skin grafts, biopsy closure, cleft-palate reconstruction, hernia repair, lymph node resection, groin repair, Caesarean section, laparoscopic trocar repair, vaginal tear repair, and hand surgery.

### Vascular and Cardiovascular Repair

The bandages and bandaging methods of the invention may be used for repairing, closing, and/or securing vascular and cardiovascular tissue. Representative procedures include coronary artery bypass grafts, coronary angioplasty, diagnostic cardia catheterization, carotid endarterectomy, and valve repair.

### Repair of Dura Tissue

Dura tissue is a fibrous membrane covering the brain and the spinal cord and lining the inner surface of the skull. Standard methods of dural repair involve the application of interrupted sutures and the use of dural replacement materials (duraplasty). This is a meticulous surgery and suffers from the limitation that pinholes produced by surgical needles can cause leakage. Moreover, intraoperative dehydration can shrink the dura creating a difficult closure since it is difficult to approximate the edges with sutures. In older patients, the dura is often more susceptible to tearing when stretched and/or sutured because the dura can be thin and fragile. Adhesives such as fibrin have been explored for repair of dura tissue, but have had limited success. See J. Latyngology and Otology 1992, 106, 356-57; Eur. J. Cardio-Thorc. Surg. 1992, 6, 52-54; Drug Intelligence and Clinical Pharmacy 1988, 22, 946-52; and Blood Reviews 1991, 5, 240-44. The sealants and methods of the present invention should be useful in repairing the dura after a craniotomy or laminectomy and prevent postoperative leakage of cerebrospinal fluid. See Neurosurgery 2003, 53, 1189-1199; and Balance, C.A. in “Some Points in the Surgery of the Brain and Its Membranes,” London, Macmillan & Co.

### Injection Site Wound

Many therapeutic agents are administered to a patient by injection. However, one complication of this procedure is that the tissue at the injection site can become infected or susceptible to poor healing. One clinical situation where infections are prone to occur is when a therapetic agent is injected into the eye of a patient. This mode of administration is used in the treatment of age-related macular degeneration (AMD) and results in about 2% of patients suffering from infection or endophthalmitis.

Age-related macular degeneration is a disease that blurs the sharp, central vision needed for “straight-ahead” activities such as reading and driving. Specifically, AMD is a progressive disease of the retina where the light-sensing cells in the central area of vision (the macula) stop working and eventually die. The disease is caused by a combination of genetic and environmental factors, and it is most common in people who are age sixty and over. In fact, AMD is the leading cause of visual impairment in the elderly population. It is estimated that fifteen million people in the United States have AMD, with approximately two million new cases diagnosed annually. There are two types of AMD—wet and dry. Wet AMD occurs when abnormal blood vessels behind the retina start to grow under the macula. These new blood vessels tend to be very fragile and often leak blood and fluid. The blood and fluid raise the macula from its normal place at the back of the eye. Damage to the macula occurs rapidly and loss of central vision can occur quickly. On the other hand, dry AMD occurs when the light-sensitive cells in the macula slowly break down, gradually blurring central vision in the affected eye. Central vision is gradually lost. In this disease, Vascular Endothelial Growth Factor (VEGF) is a key growth factor, which promotes the growth of new blood vessels. Currently, it is believed that that when the retinal pigment epithelial (RPE) cells begin to wither from lack of nutrition (i.e., ischemia), VEGF is up-regulated and new vessels are created. Yet, the vessels do not form properly and leaking results. This leakage causes scarring in the macula and eventual loss of central vision. To prevent or inhibit this neovascularization process, antiangiogenic drugs are given the patient. In most cases, the drugs are injected into the vitreous of the eyeball, then pass into the subretinal space where the vessels proliferate. These drugs include mucagenm squalamine lactate, combretastatin 4 prodrug, and avastin.

The sealants and methods of the present invention should be useful in sealing injection site wounds. Among the various possibilities, the injection can be given and then the sealant applied to the injection site, or alternatively the sealant can be applied and then the injection can be done through the sealant.

### Therapeutic Use of Crosslinked Polyalkyleneimines

To date poly alkyleneimines (PAIs) have been used primarily as gene transfection agents with limited success. In general, large PAIs (25,000 molecular weight and higher) are more efficient at forming complexes and condensing with polynucleic acids, but their associated toxicity has also been reported to increase with increasing molecular weight. As a strategy to reduce this toxicity, polyalkylene glycols (PAGs), such as monomethoxy-polyethylene glycols, have been grafted to the PAIs in vitro before condensation with polynucleic acids. In a few cases, PAIs have been combined with difunctionally activated PEG in dilute solution to produce linear block copolymers of PAI and PAG, or in an emulsion polymerization process to produce small PAI/PAG microspheres. In both of these cases, the PAI/PAG block copolymers were synthesized in vitro for the purpose of condensing with polynucleic acids for gene transfection.

### SUMMARY

One aspect of the present invention relates to methods of bandaging, covering, or bridging a defect, a wound, or a void in the tissue of a patient, using a two component system composed of masking material, which creates an anti-adhesion barrier, and a covering material. In certain embodiments, the covering material comprises an in situ polymerizing sealant. In certain embodiments, the in situ polymerizing sealant is a hydrogel which binds to the healthy tissue but remains unadhered to the area under the masking material. In certain embodiments, the masking material is also a hydrogel. In certain embodiments, normal biological processes may dissolve away the masking material leaving a protective cover of polymerized sealant over the defect, wound, or void.

### BRIEF DESCRIPTION OF FIGURES

FIG. 1 depicts one embodiment of the two component system of the invention.

FIG. 2 is a scheme showing a general depiction of PEI chemistry.

FIG. 3 depicts ureido, urea and acetoacetoxy moieties.

FIG. 4 depicts a scheme showing partial neutralization of free amines in polyamines.

FIG. 5 depicts schemes showing polymerization of acrylamide and acrylic acid, and subsequent modifications.

FIG. 6 depicts a double-acting, single-barrel syringe.

FIG. 7 depicts a double-barrel syringe.

FIG. 8 depicts poly amines that may be reacted with electrophile-bearing polyalkylene glycols to form a hydrogel.

FIG. 9 depicts poly alkyleneimines that may be reacted with electrophile-bearing polyalkylene glycols to form a hydrogel.

FIG. 10 depicts poly amines that may be reacted with electrophile-bearing polyalkylene glycols to form a hydrogel.

FIG. 11 depicts activated polyethylene glycols that may be reacted with nucleophile-bearing poly alkyleneimines to form a hydrogel, wherein variable w is an integer in the range of about 5 to about 200.

FIG. 12 depicts activated polyethylene glycols that may be reacted with nucleophile-bearing poly alkyleneimines to form a hydrogel, wherein variable w is an integer in the range of about 5 to about 200.

FIG. 13 depicts activated poly alkyleneimines that may be reacted with nucleophile-bearing polyalkylene glycols, e.g., PEG-(NH₂)₂, to form a hydrogel; wherein variables x, y, and z each represent an integer in the range of about 2 to about 200.

FIG. 14 depicts nucleophile-bearing polyethylene glycols that may be reacted with electrophile-bearing poly alkyleneimines to form a hydrogel, wherein variable w is an integer in the range of about 5 to about 200.

FIG. 15 depicts nucleophile-bearing polyethylene glycols that may be reacted with electrophile-bearing poly alkyleneimines to form a hydrogel, wherein variable w is an integer in the range of about 5 to about 200.

FIG. 16 depicts poly alkyleneimines containing acrylate groups for use in photopolymerization procedures.

FIG. 17 depicts poly alkyleneimines containing methacrylate groups for use in photopolymerization procedures.

FIG. 18 depicts [A] polyalkyleneimine G1 DAB-PPI, a first generation PPI dendrimer with DAB (diaminobutane) as core; and [B] polyalkyleneimine G2 DAB-PPI, a second generation PPI dendrimer with DAB (diaminobutane) as core.

FIG. 19 tabulates various PAI and activated PAG combinations (or derivatized PAG combinations) that have been used to make various hydrogels. In the table the following abbreviations are used: Polypropylene Glycol (PPG); Polyethylene Glycol (PEG); Amino Succinimidyl Glutarate (ASG); Succinimidy Propionic Acid (SPA); Succinimidy Glutarate (SG); Succinimidyl α-Methyl Butanoic Acid (SMB); Succinimidy 3-Methyl Glutarate (S3MG); Succinimidy 3,3-Dimethyl Glutarate (S3,3DMG); polyethyleneimine (PEI); First Generation polypropyleneimine dendrimer with diaminobutane core [G1-PPI(DAB)].

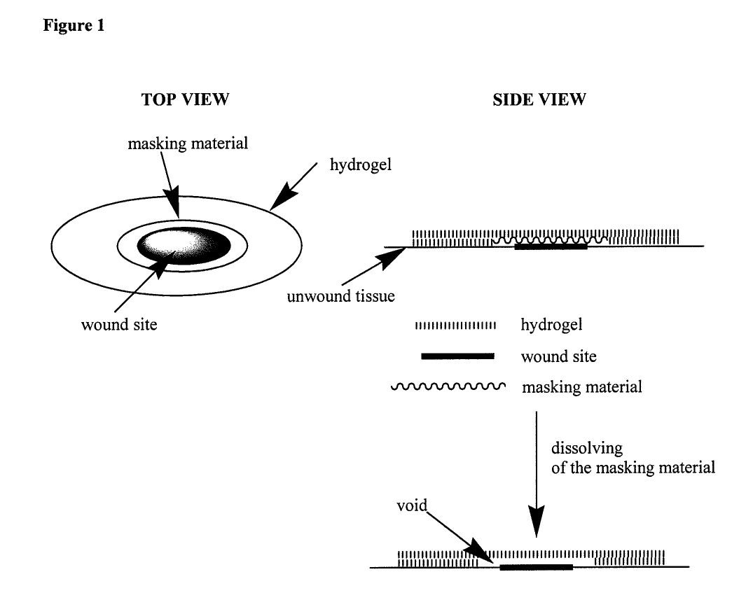

### DETAILED DESCRIPTION

Remarkably, polymeric masking materials have been invented that do not adhere to an underlying defect, wound, or void in the tissue of a patient. In certain embodiments, the polymeric masking materials may be placed on a defect, wound, or void, followed by application of a mixture that polymerizes to give a film. In certain embodiments, said mixture that polymerizes to give a film is applied as a spray.

In certain embodiments, the masking material is applied to the defect, wound, or void in the tissue of a patient and may optionally extend on to healthy (e.g., unwounded) tissue to ensure that the defect, wound, or void is completely covered. In addition, the masking material can be optionally impregnated with a visualization agent, so one can determine how much area has been covered by the masking material. In certain embodiments, after application of the masking material, an in situ polymerizing sealant may be applied to cover an area around the wound site such that the masking material is covered and unwounded tissue is covered. In certain embodiments, the in situ polymerizing sealant is a hydrogel which binds to healthy tissue but does not adhere to the area under the masking material. In certain embodiments, normal biological processes may degrade and/or absorb and excrete the masking material, leaving a protective cover of polymerized sealant over the wound site. The degradation rate of the in situ polymerizing sealant may be adjusted to be suitable for the healing rate of the underlying tissue.

The bandages formed by the use of the masking and covering compositions of the invention are applicable to sealing a large variety of defects, wounds, and voids. For example, the compositions of the invention may be used in ophthalmic applications, cardiovascular surgery, urinary tract surgery (nephrotomy closure, urethral repair, hypospadia repair), pulmonary surgery (sealing parenchymal & bronchial leaks, bronchopleural fistula repair, persistent air leak repairs), GI tract and stomach surgery (parotid cutaneous fistula, tracheo-oesophageal fistula, peptic ulcer repair), joint surgery (cartilage repair, meniscal repair), heart surgery (cardiac ventricular rupture repair), brain surgery (dural defect repairs), ear surgery (ear drum perforation), alveolar osteitis (“dry socket”) and related post-surgical oral indications, and post-surgical drainage reduction (mastectomy, auxiliary dissection).

In addition, the masking and covering compositions of the present invention can be used along with suture or staples to close or secure a wound. These wounds include those caused by trauma, surgical procedure, infection, or a health condition. When used in this manner, the covering composition may provide a leak tight barrier for liquids or air.

In certain embodiments, the compositions and methods described herein may be useful in neurosurgery, e.g., in dural closure. Specifically, while it is known that some in situ polymerizing systems may be sprayed over dural defects, these materials may adhere to the underlying pia mater and cortex (brain). However, the use of an anti-adhesion barrier on these underlying structures will prevent undesirable adhesion(s), thereby allowing an in situ polymerizing film to be formed over the defect and in contact with exposed dura to create a fast-forming, water-tight seal.

In certain embodiments, a biocompatible liquid or solution is used as the masking material agent, which is applied to the effected area via brush or spray application. Examples of masking materials suitable for use include materials which comprise PVA solutions, PEG solutions, water soluble lubricants, hyaluronic acid, or combinations thereof. In certain embodiments, in order avoid over spray or excess masking material, a sheet of sterile material can be cut such that when it is placed over the wound site that only the wound site is exposed, while leaving the remaining surgical site covered. The masking material can then be applied to the desired area. After removal of this barrier, the area surrounding the covering material (e.g., a hydrogel) will be unadulterated.

In certain embodiments, an in situ polymerizing polymer system may be used as the masking material, which will rapidly degrade/dissolve after application of the more durable covering material (e.g., a hydrogel). In such embodiments, a PEG-Succinimidyl Succinate based hydrogel or a PEG-Succinimidyl Glutarate based hydrogel may be used as the masking material. In both instances, there are at least two active esters present on the PEG based polymer and the PEG based polymer is crosslinked with another small molecule or polymer component with at least two nucleophilic groups capable of reacting with the activated esters on the PEG, at least one of the components must have three or more reactive groups. The application of such a hydrogel to the wound site would provide a more static situation for the application of the more durable covering material, in that the rapidly degradable masking material gel would reduce the flow of fluids from the wound site and therefore allow for better adhesion of the covering material resulting in a stronger bandage.

In another such embodiment of a PEG based hydrogel as an in situ polymerizing masking material, there are at least two active esters present on the PEG based polymer and the PEG based polymer is crosslinked with a polyalkyleneimine with at least three nucleophilic groups capable of reacting with the activated esters on the PEG.

In another such embodiment of a PEG based hydrogel as an in situ polymerizing masking material, there are at least two active esters present on the PEG based polymer and the PEG based polymer is crosslinked with another small molecule or polymer component containing secondary and tertiary amines with at least two nucleophilic groups capable of reacting with the activated esters on the PEG, at least one of the components must have three or more reactive groups.

In certain instances, the masking material and/or the covering material is a polyalkyleneimine-containing hydrogel. In certain embodiments, the polyalkyleneimine is polyethyleneimine. Treatment of the polyethyleneimine with a cross-linking reagent causes the polyethyleneimine polymers to polymerize forming a seal. In certain instances, the cross-linking reagent is a polyethylene glycol having reactive terminal groups. In certain instances, the reactive terminal groups are activated esters, such as N-hydroxy succinimide ester. In certain instances, the reactive terminal groups are isocyanates. In certain instances, the polyethyleneimine has a lysine, cysteine, isocysteine or other nucleophilic group attached to the periphery of the polymer. In certain instances, the polyethyleneimine is mixed with a second polymer, such as a polyethylene glycol containing nucleophilic groups. In certain instances, the compositions used to seal the wound or tissue plane are formed by reacting a polyalkyleneimine bearing electrophilic groups with a cross-linking reagent containing nucleophilic groups. In certain instances, the electrophilic groups on the polyalkyleneimine are activated esters, such as N-hydroxy succinimide ester. In certain instances, the compositions used to seal the wound or tissue plane are formed by reacting a polyalkyleneimine bearing photopolymerizable groups with ultraviolet or visible light. Importantly, compositions used as covering materials which contain PEI or a derivative of PEI are found to adhere tightly to the tissue. In certain instances, the covering material is attached to mammalian tissue.

### Hydrogel Dressings

Hydrogel dressings are semipermeable to gases and water vapor. Note that certain hydrogel dressings may contain polyurethane and thus, to a certain extent, have occlusive properties. However, one of the unique features of hydrogels (as distinguished from other dressings) is due to the presence of hydrophylic polymers in their content: The amorphous gel formed maintains a moist and hydrated environment.

Hydrogels can be produced from either natural or synthetic polymers. Natural polymers include, for example, dextran reduced with sodium borohydride and crosslinked with epichlorohydrin. Other natural polymers include, for example, keratin derivatives, glucoaminoglycans and collagen. Synthetic polymers which may be used for the production of hydrogels include, for example, polyethylene oxide and block copolymers of hydroxyl terminated propylene and ethylene oxides. Other synthetic polymers which may be used include composites of poly acrylamide and polyurethane, poly vinyl alcohols and poly 2-hydroxyethyl methacrylate (HEMA).

Further, the hydrogels of the invention may be made up of a three-dimensional matrix of hydrophylic polymers, such as the polyalkyleneimines, carboxymethylcellulose (Intrasite Gel®) or polyethylene oxide (Vigilon®), combined with a high water content. In certain embodiments, hydrogel preparations may also contain glycerin and/or pectin. In certain embodiments, hydrogels may be available in sheet form, or as a spreadable viscous gel.

Examples of commercial hydrogel dressings which may be used in the present invention include, but are not limited to, Aquaflo® (Kendall), Aquasorb® (Deroyal), Carrasyn Gel Wound Dressing® (Carrington Laboratores), Curafil® (Kendall), Cutinova Gel® (Beiersdorf-Jobst), Dermagran Hydrogel Zinc-Saline Wound Dressing® (Derma Sciences), Duoderm hydroactive Gel® (Convatec), Hydrosorb® (Hartmann), Hyfil wound Gel® (B. Braun Medical), HyperGel® (Molnlycke Health Care) Iamin hydrating Gel® (Smith & Nephew), Macropro Gel® (Brennen Medical), MPM Excel Gel® (MPM Medical), Purilon Gel® (Coloplast), StenGel® (Seton Scholl) and Viglion® (Bard Medical Division).

### Polyalkyleneimine Hydrogels

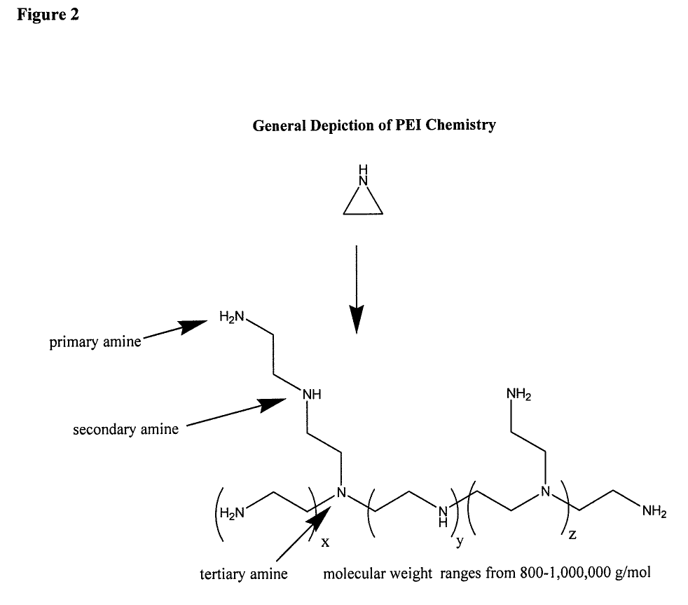

In one aspect of the present invention, the masking material, the covering material, or both, comprise polyalkyleneimine hydrogels. These gel are prepared by reacting a polyalkyleneimine (PAI) with a cross-linking agent, such as an activated polyethylene glycol. The gels of the invention are amendable to a variety of clinical treatments, such incisions created during general surgery or wounds/incisions in the dura during neurosurgery. The polyalkyleneimine gels of the invention offer the advantage that the secondary and tertiary amino groups of the gel can be converted to secondary and tertiary ammonium cations which may encourage cell attachment and cell ingrowth. In certain instances, the secondary and tertiary amines of the polyethyleneimine (PEI) can be converted to ammonium cations by placing the PEI in an aqueous solution. For example, see FIG. 2.

The polyalkyleneimine (PAI) gels of the invention have superior adhesion properties. Their superior tissue-adhesion properties may be due to two factors. First, the cationic properties of PEI promote interaction with, and possibly penetration within, an anionic tissue substrate. See Rep. Prog. Phys. 1998, 61, 1325-1365. Cationic interactions could occur through the secondary and tertiary ammonium cations of the PEI backbone or through primary amino groups that did not react with the cross-linking reagent. Second, PEI contains a large number of functional groups per molecule, thus promoting an increased number of crosslinkable sites within the polymer network. The increased number of crosslinkable sites within the polymer network affords dense, interpenetrating networks between the hydrogel and the tissue surface. The number of free amino groups in the hydrogel can be controlled by varying the ratio of PEI to activated PEG. The ability to control the number of free amino groups is significant because greater cell ingrowth was observed in tissue ingrowth experiments using hydrogels that contained a larger percentage of PEI.

In addition to increased adhesion, it has been found that as the molecular weight of the PEI increases from about 1,300 to about 2,000 g/mol the swelling of the resulting hydrogel decreases in certain instances. Thus, the molecular weight of the PEI may be adjusted in order to tune the swelling-effects of the resultant hydrogel.

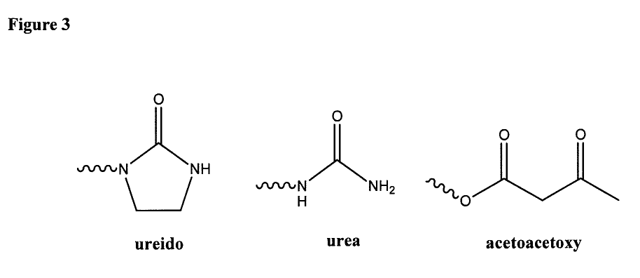

A large variety of PAI derivatives are amenable to the present invention. For example, the amino groups of the PAI may be functionalized with a fatty acid, lower alkyl, an alkenyl, or alkynyl group. In addition, the amino groups or a portion of the amino groups may be functionalized to contain active agents, pharmaceutical agents, preservatives, radio isotopic ions, magnetically detectable ions, antibodies, medical contrast agents, colorants, dyes, or other visualization agents. In certain instances, about 1% to about 70% of the primary amines of the PEI are functionalized. The PAI derivatives may contain hydrolytically and/or enzymatically degradable linkages capable of releasing the functional derivatives, active agents, pharmaceutical agents, preservatives, radio isotopic ions, magnetically detectable ions, antibodies, colorants, dyes, or other visualization agents. Alternatively, a different nucleophile can be added to the PEI, such as a cysteine, isocysteine, thiol, or other such nucleophilic group. For example, a PEI can be modified such that all the primary amines are modified with a cysteine thus affording a PEI derivative which can form crosslinked gel/networks using the amine, thiol, or both the amine and thio. In certain instances, an ureido, urea, acetoacetoxy, RGD peptide, EDTA, or carbohydrate group may be bonded to one or more of the amino groups of the PEI. Representative carbohydrates include erythrose, threose, ribose, arabinose, xylose, lyxose, allose, altrose, glucose, mannose, gulose, idose, galactose, talose, sucrose, lactose, and the like. It is possible that the ureido group and urea group will impart adhesion partially via a cation/anion interaction. The acetoacetoxy group may adhere to tissue by making a metal complex on the surface of the tissue. See FIG. 3.

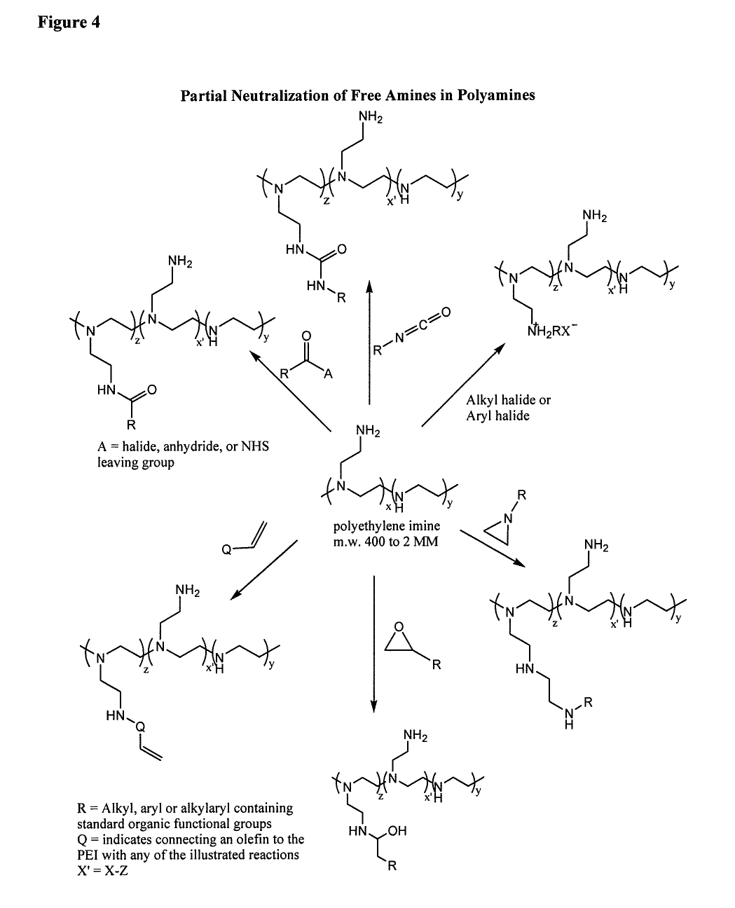

In certain instances, the PEI is functionalized so that both primary amino (—NH₂) groups and thiol (—SH) groups could react with electrophilic groups or a combination of them, such as an acrylate, succinimidyl ester, maleimide, ester, or aldehyde. The electrophilic groups can be attached to poly(alkyleneoxide) (e.g., PEG, PPG or PEG-PPG) polymers. Two or more electrophilic groups are required. Of course, the degree of PEI functionalization may be varied in order to obtain the desired physical properties of the resultant gel. In certain instances, only about 1% of the primary amino groups of the PEI are functionalized. In other instances, about 5% to about 25% of the primary amino groups of the PEI are functionalized. In other instances, about 25% to about 50% of the primary amino groups of the PEI are functionalized. In other instances, about 99% of the primary amino groups of the PEI are functionalized. In certain instances, one or more of the amino groups are reacted with an epoxide or acylating agent. In certain instances, one or more of the amino groups are reacted with an isocyanate, as shown in FIG. 4.

The molecular weight of the PEI may be adjusted to tune the physical properties of the gel formed by addition of the cross-linking agent. In certain instances, the PEI has a weight average molecular weight of about 400 g/mol to about 2,000,000 g/mol. In certain instances, the PEI has a weight average molecular weight of about 400 g/mol to about 1,000,000 g/mol. In certain instances, the PEI has a weight average molecular weight of about 400 g/mol to about 500,000 g/mol. In certain instances, the PEI has a weight average molecular weight of about 400 g/mol to about 100,000 g/mol. In certain instances, the PEI has a weight average molecular weight of about 400 g/mol to about 50,000 g/mol. In certain instances, the PEI has a weight average molecular weight of about 400 g/mol to about 10,000 g/mol. In certain instances, the PEI has a weight average molecular weight of about 400 g/mol to about 5,000 g/mol. In certain instances, the PEI has a weight average molecular weight of about 400 g/mol to about 2,000 g/mol.

In certain instances, the polyalkyleneimine has a weight average molecular weight of about 600 to about 10,000 Daltons, the polyalkylene glycol has a weight average molecular weight of about 500 to about 20,000 Daltons, and the molar ratio of the polyalkyleneimine to the polyalkylene glycol is within a molar range of about 0.025:1 to about 0.4:1. In certain instances, the hydrogel reaches equilibrium swelling in about 5 to about 30 hours. In certain instances, the hydrogel reaches equilibrium swelling in about 18 hours.

In certain instances, the aforementioned polyalkyleneimine/polyalkylene glycol hydrogels may be used or modified to non-covalently carry or contain active agents, pharmaceutical agents, preservatives, radio isotopic ions, magnetically detectable ions, antibodies, medical contrast agents, colorants, dyes, or other visualization agents.

Many prior sealant systems are not optimal because the sealant system may degrade before appreciable healing or tissue ingrowth occurs. For example, tissue ingrowth often begins within one week after application of the sealant, and complete tissue ingrowth may occur within 28 days after application of the sealant in very porous systems. However, many prior sealant systems contain degradable linkages which can cause the hydrogels to degrade before appreciable tissue ingrowth occurs. While use of these materials alone is not advantageous, these materials may be used as masking materials. Accordingly, in certain instances, when polyalkyleneimine hydrogel are used as covering materials the covering can maintain its mechanical strength for at least about 7 days. In certain instances, the polyalkyleneimine hydrogel sealants of the invention maintain mechanical strength for at least about 20 days. This rate of degradation allows the masking material to degrade, while keeping the covering material in place.

Since charged species encourage tissue growth, polyalkyleneimines as masking material are advantageous because they allow for incorporation of a large number of charged species. The charged species are created by converting unreacted primary amines, and internal secondary and tertiary amines into ammonium cations under physiological conditions. Table 1 below illustrates the number of primary, secondary and tertiary amines contained in various crosslinkers based on a polymer system having eighteen primary amines. As illustrated in Table 1, the trilysine crosslinker contains only primary amines and a pendant carboxylate while a PPI(DAB)-G1 dendrimer adds 9 units of potential cationic charge with the addition of 9 tertiary amines. The PEI₈₀₀ adds 14 units of potentially charged species (i.e., 155% more charge) compared to the PPI(DAB)-G1 dendrimer, while the PEInoo adds 26% more potentially charged species than PEI₈₀₀. Finally, PEI₂₅₀₀₀ adds 24% more potentially charged species than PEI₂₀₀₀, owing to the increased number of secondary and tertiary amines. Since the number of secondary and tertiary amino groups increases with increasing molecular weight of the polyalkyleneimine, the polyalkyleneimine hydrogels of the invention can be tuned by incorporating crosslinkers with varying molecular weights, and hence charge density, in order to affect the tissue ingrowth and degradation properties of the hydrogel.

TABLE 1

| Crosslinker | 1° amines | 2° amines | 3° amines |
|---|---|---|---|
| PEI₂₅₀₀₀ | 18 | 22 | 14 |
| PEI₂₀₀₀ | 18 | 17 | 12 |
| PEI₈₀₀ | 18 | 14 | 9 |
| PPI(DAB)-G1 | 18 | 0 | 9 |
| Trilysine | 4 | 0 | 0 |

Again, when used as masking material, polyalkyleneimine hydrogel sealants offer an advantage over prior sealant systems because polyalkyleneimines, especially derivatized polyalkyleneimines, should have antimicrobial and antiviral activity. Recent reports indicate that both polyalkyleneimines and derivatives thereof have antimicrobial properties, while lacking activity against mammalian cells. See Biotechnol. Bioeng. 2005, 90, 715-722; Biotechnol. Bioeng. 2003, 83, 168-172; Biotechnology Letters 2003, 25, 1661-1665; Biotechnol. Prog. 2002, 18, 1082-1086; Chem. Commun. 1999, 1585-1586; and Proc. Nat. Acad. Sci. USA 2006, 103, 17667-17671. Thus, hydrogels prepared from polyalkyleneimines may help fight, inhibit, prevent or even eliminate the chance for infection when applied to the tissue of a patient. Since the presence of cationic groups, especially quaternary amines, may influence the antimicrobial properties of the hydrogel, the PAI, in certain instances, may be derivatized with one or more quaternary amines. In certain instances, the PAI may be derivatized with four or more quaternary amines. In certain instances, the PAI may be derivatized with ten or more quaternary amines. Since the presence of cationic groups and hydrophobic side chains, when combined, tend to confer better antimicrobial properties, the PAI, in certain instances, may be derivatized with one or more quaternary amines and one or more fatty acid, lower alkyl, alkenyl, or alkynyl groups.

Polyalkyleneimine hydrogels as masking material and covering material offer the additional advantage that the amino groups of the polyalkyleneimine can act as a buffering agent. The ability to control the pH during preparation of the hydrogel is important because certain pHs are optimal for crosslinking of the components. In particular, the pH of a mixture of crosslinking components can affect the rate at which the crosslinking reaction takes places. In some instances, the desired pH can be achieved by adding a buffering agent, such as phosphates, carbonates, borates, and the like, to the solution containing the crosslinking components. However, when using poly alkyleneimines as a crosslinkable component, the primary, secondary, and tertiary amines act as buffering agents to provide some buffering capacity throughout a wide range of pHs. See Bioorganic Chemistry 1994, 22, 318-327. Moreover, as the crosslinkable component reacts, some of the amines are removed from solution, thereby reducing the pH. Since quick set-times can require higher pHs, it is advantageous to use a crosslinkable component which influences the pH so that the pH will lower to more physiological levels soon after mixing. This buffering feature of polyalkyleneimines eliminates the need for a strong buffer to achieve the high pH-levels sometimes used in preparing a hydrogel. Notably, addition of strong buffers may not be desirable because such buffers may remain in the sealant and cause the patient's tissue to become irritated.

### Other Amine-Containing Materials Suitable for Use as Masking/Covering Materials

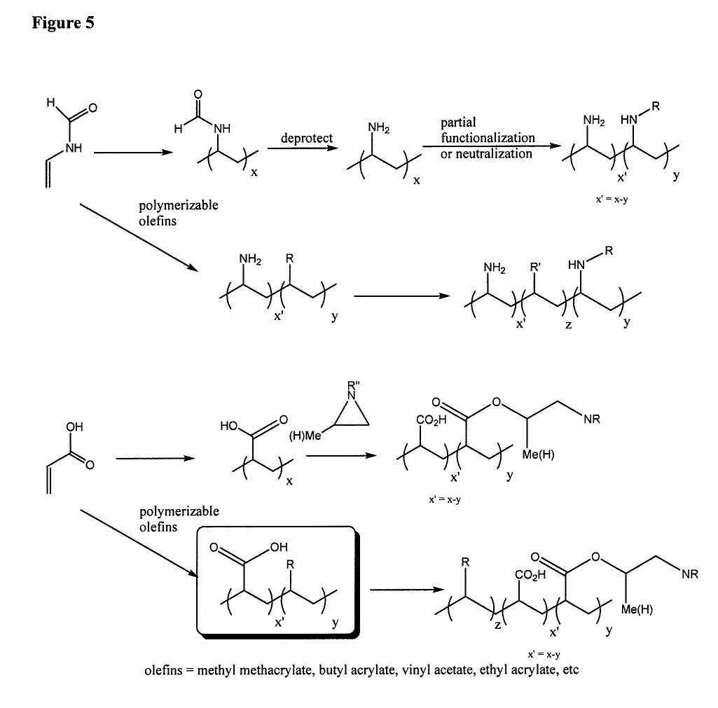

The methods of the invention are also amenable to other types of amine-containing masking materials and covering materials. For example, polymerization of acrylamide, followed by partial or complete conversion of the amide groups to amino groups, would provide a polyamine. Likewise, copolymerization of acrylamide with another monomeric olefin could be used to tune the properties of the resultant polyamine. Similarly, polymerization of acrylic acid, followed by partial or complete conversion of the carboxylic acids to amino groups, or partial or complete reaction of the carboxylic acid with an aziridine would provide a polyamine. In addition, copolymerization of acrylic acid with an olefin, followed by conversion of the carboxylic acid to an amine-containing moiety would provide a polyamine. In certain instances, a polylysine or polylysine copolymer may be used in the methods of the present invention. See FIG. 5.

### Cross-Linking Agents

In certain embodiments, the masking material and covering material of the invention may be formed by reacting a polyalkyleneimine, or other amine-containing polymer, with a cross-linking agent. A large number of cross-linking agents are amenable to the invention. In certain instances, the cross-linking agent is an activated polyethylene glycol. The activating group is preferably an electrophilic group. For example, in certain instances, the polyethylene glycol contains a N-hydroxysuccinimide group at each end of the polymer. In certain instances, the succinimide is functionalized with a sulfonic acid moiety. In certain instances, the polyethylene glycol contains an aldehyde at each end of the polyethylene glycol. In certain instances the polyethylene glycol is a star, dendritic, or branched polymer with three or more activating groups.

In certain instances, the polyethylene glycol cross-linking agent contains two or more different electrophiles. The different electrophiles may have similar or dissimilar reactivities. The different electrophiles provide linkages having similar or dissimilar degradation rates. The selection of electrophiles allows for control over the crosslinking reactions to form the hydrogels, the adhesive properties, and the degradation rate of the formed hydrogel. For example, a polyethylene glycol can be derivatized such that one end of the polyethylene glycol contains a SPA and another end contains a SG. In this example, both are activated esters, but the degradation rates of the two linkages are different. For example, a hydrogel prepared with only a PEG-SPA is generally stable at 37° C. for more than about four months, whereas a hydrogel prepared with PEG-SG is often stable for less than about one week. Notably, one hydrogel prepared from PEI and a PEG-SPA/SG having a 60:40 ratio of SPA:SG degraded in about a week.

In certain instance, more than one polyethylene glycol cross-liking agents can be used. For example, a mixture of PEI/PEG-SPA and PEI/PEG-SG. The different cross-linkers may provide linkages having similar or dissimilar degradation rates, and thus the properties of the resulting hydrogel can be controlled.

In certain instances, the polyethylene glycol cross-linking agent contains a hydrophobic moiety. In certain instances, alkyl groups are installed between the polyethylene glycol and the terminal electrophilic groups of the cross-linking agent. In certain instances, the alkyl group contains about 4 to about 30 carbon atoms. In certain instances, the alkyl group contains about 5 to about 15 carbon atoms. In certain instances, the hydrophobic moiety is an aryl or aralkyl group. In certain instances, the alkyl moiety of the aralkyl group contains between 5-10 carbon atoms.

In certain instances, the polyethylene glycol cross-linking agent is represented by the generic formula (ii) below, wherein w is an integer in the range of about 5 to 10,000, and n is an integer in the range of about 5 to about 30.

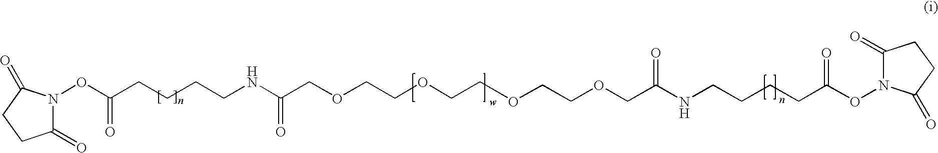

In certain instances, the polyethylene glycol cross-linking agent is represented by the generic formula (ii) below, wherein w is an integer in the range of about 5 to 10,000, and m is an integer in the range of about 1 to about 50.

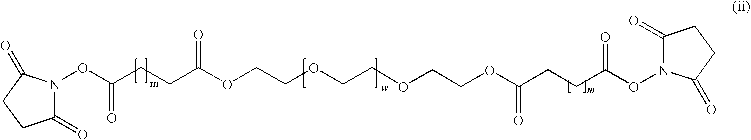

In certain instances the hydrophobic moiety may be used as a foaming agent. The linkages between the polyethylene glycol and the hydrophobic moiety can be esters, amides, carbamates, carbonates, urea, urethane, and so forth.

A further embodiment of this invention is the use of a chemical peptide ligation reaction to create a crosslinked gel involving a dendritic polymer. In this reaction an aldehyde, aldehyde-acid or aldehyde-ester reacts with a cysteine-functionalized polymer to form a gel or crosslinked network. In certain instances, the dendritic polymers have nucleophilic groups, such as primary amino groups or thiol groups, which can react with electrophilic groups, such as an acrylate, succinimidyl ester, maleimide, ester aldehyde, or aldehyde on a small molecule. In certain instances, the dendritic polymer has nucleophilic groups capable of reacting with an activated diester of sebacic acid.

### Methods of the Invention

One aspect of the invention relates to a method of bandaging, covering, or bridging a defect, a wound, or a void in the tissue of a patient, comprising the steps of:

covering the defect, wound, or void with a first material; and

covering the first material with a second material;

wherein the area covered by the first material is greater than or equal to the area of the defect, wound, or void; the second material covers the area covered by the first material; and the area covered by the second material is greater than the area covered by the first material.

In certain embodiments, the present invention relates to any one of the aforementioned methods, wherein the defect, wound, or void is located in the dura.

In certain embodiments, the present invention relates to any one of the aforementioned methods, wherein the defect, wound, or void is in the dura matter.

In certain embodiments, the present invention relates to any one of the aforementioned methods, wherein the first material is brushed onto the defect, wound, or void.

In certain embodiments, the present invention relates to any one of the aforementioned methods, wherein the first material is sprayed onto the defect, wound, or void.

In certain embodiments, the present invention relates to any one of the aforementioned methods, wherein the first material is applied via a cannula onto the defect, wound, or void.

In certain embodiments, the present invention relates to any one of the aforementioned methods, wherein the first material is biodegradable.

In certain embodiments, the present invention relates to any one of the aforementioned methods, wherein the first material is substantially degraded, displaced, or diluted faster than the second material.

In certain embodiments, the present invention relates to any one of the aforementioned methods, wherein the first material is substantially degraded, displaced, or diluted in about 30 minutes.

In certain embodiments, the present invention relates to any one of the aforementioned methods, wherein the first material is substantially degraded, displaced, or diluted in about 2 hours.

In certain embodiments, the present invention relates to any one of the aforementioned methods, wherein the first material is substantially degraded, displaced, or diluted in about 12 hours.

In certain embodiments, the present invention relates to any one of the aforementioned methods, wherein the first material is substantially degraded, displaced, or diluted in about 24 hours.

In certain embodiments, the present invention relates to any one of the aforementioned methods, wherein the first material is poly(lactic acid), poly(glycolic acid), or a copolymer thereof.

In certain embodiments, the present invention relates to any one of the aforementioned methods, wherein the first material comprises collagen, hyaluronic acid, albumin, cellulose, elastin, fibrin, fibronectin, gelatine, heparin, heparin sulfate, polylysine, poly(vinyl acetate), polyvinylpyrrolidone, poly(acrylic acid), poly(ethylene glycol), poly(propylene glycol)-poly(ethylene glycol) copolymer, trimethylene carbonate, or a polypeptide comprising the tripeptide Arg-Gly-Asp.

In certain embodiments, the present invention relates to any one of the aforementioned methods, wherein the first material is a hydrogel.

In certain embodiments, the present invention relates to any one of the aforementioned methods, wherein the first material is a pre-formed hydrogel.

In certain embodiments, the present invention relates to any one of the aforementioned methods, wherein the first material is a polyalkyleneimine-containing hydrogel.

In certain embodiments, the present invention relates to any one of the aforementioned methods, wherein the first material is a hydrogel; and the hydrogel has pores in the range of about 1 micron to about 100 microns in diameter.

In certain embodiments, the present invention relates to any one of the aforementioned methods, wherein the first material comprises a medicament, a colorant, a flavoring, a scent, a fibrous additive, a thickener or a plasticizer.

In certain embodiments, the present invention relates to any one of the aforementioned methods, wherein the first material has a sterility assurance level of at least about 10⁻³.

In certain embodiments, the present invention relates to any one of the aforementioned methods, wherein the first material has a sterility assurance level of at least about 10⁻⁴.

In certain embodiments, the present invention relates to any one of the aforementioned methods, wherein the first material has a sterility assurance level of at least about 10⁻⁵.

In certain embodiments, the present invention relates to any one of the aforementioned methods, wherein the first material has a sterility assurance level of at least about 10⁻⁶.

In certain embodiments, the present invention relates to any one of the aforementioned methods, wherein the second material is brushed onto the defect, wound, or void.

In certain embodiments, the present invention relates to any one of the aforementioned methods, wherein the second material is sprayed onto the defect, wound, or void.

In certain embodiments, the present invention relates to any one of the aforementioned methods, wherein the second material is applied via a cannula onto the defect, wound, or void.

In certain embodiments, the present invention relates to any one of the aforementioned methods, wherein the second material is biodegradable.

In certain embodiments, the present invention relates to any one of the aforementioned methods, wherein the second material is a hydrogel.

In certain embodiments, the present invention relates to any one of the aforementioned methods, wherein the second material is a pre-formed hydrogel.

In certain embodiments, the present invention relates to any one of the aforementioned methods, wherein the second material is a polyalkyleneimine-containing hydrogel.

In certain embodiments, the present invention relates to any one of the aforementioned methods, wherein said the second material is a hydrogel; and said hydrogel has pores in the range of about 1 micron to about 100 microns in diameter.

In certain embodiments, the present invention relates to any one of the aforementioned methods, wherein the second material comprises a medicament, a colorant, a flavoring, a scent, a fibrous additive, a thickener or a plasticizer.

In certain embodiments, the present invention relates to any one of the aforementioned methods, wherein the second material has a sterility assurance level of at least about 10⁻³.

In certain embodiments, the present invention relates to any one of the aforementioned methods, wherein the second material has a sterility assurance level of at least about 10⁻⁴.

In certain embodiments, the present invention relates to any one of the aforementioned methods, wherein the second material has a sterility assurance level of at least about 10⁻⁵.

In certain embodiments, the present invention relates to any one of the aforementioned methods, wherein the second material has a sterility assurance level of at least about 10⁻⁶.

In certain embodiments, the present invention relates to any one of the aforementioned methods, further comprising the step of placing a piece of sterile material around the defect, wound, or void, so that only the defect, wound, or void is exposed, prior to placing the first material over the defect, wound, or void.

In certain embodiments, the present invention relates to any one of the aforementioned methods, further comprising the step of applying a dissolvable polymer or inorganic salt to the wound, void, or tissue of a patient.

In certain embodiments, the present invention relates to any one of the aforementioned methods, further comprising the step of applying a mesh to the wound, void, or tissue of a patient.

In certain embodiments, the present invention relates to any one of the aforementioned methods, further comprising the step of applying a mesh to the wound, void, or tissue of a patient; wherein the mesh comprises methylmethacrylate, mersilene, silicone, Teflon®, Dacron®, polyethylene, polyester, titanium-Dacron®, hydroxylapatite, or combinations thereof.

In certain embodiments, the present invention relates to any one of the aforementioned methods, further comprising the step of applying a mesh to the wound, void, or tissue of a patient; wherein the mesh comprises polypropylene or polyester.

In certain embodiments, the present invention relates to any one of the aforementioned methods, further comprising the step of applying a mesh to the wound, void, or tissue of a patient; wherein the mesh comprises a biodegradable polymer.

In certain embodiments, the present invention relates to any one of the aforementioned methods, further comprising the step of applying a mesh to the wound, void, or tissue of a patient; wherein the mesh comprises poly(glycolic acid), poly(lactic acid), or copolymers thereof.

In certain embodiments, the present invention relates to any one of the aforementioned methods, wherein the first material comprises a mesh.

In certain embodiments, the present invention relates to any one of the aforementioned methods, wherein the first material comprises a mesh; and the mesh comprises methylmethacrylate, mersilene, silicone, Teflon®, Dacron®, polyethylene, polyester, titanium-Dacron®, hydroxylapatite, or combinations thereof.

In certain embodiments, the present invention relates to any one of the aforementioned methods, wherein the first material comprises a mesh; and the mesh comprises polypropylene or polyester.

In certain embodiments, the present invention relates to any one of the aforementioned methods, wherein the first material comprises a mesh; and the mesh comprises a biodegradable polymer.

In certain embodiments, the present invention relates to any one of the aforementioned methods, wherein the first material comprises a mesh; and the mesh comprises poly(glycolic acid), poly(lactic acid), or copolymers thereof.

In certain embodiments, the present invention relates to any one of the aforementioned methods, wherein the second material comprises a mesh.

In certain embodiments, the present invention relates to any one of the aforementioned methods, wherein the second material comprises a mesh; and the mesh comprises methylmethacrylate, mersilene, silicone, Teflon®, Dacron®, polyethylene, polyester, titanium-Dacron®, hydroxylapatite, or combinations thereof.

In certain embodiments, the present invention relates to any one of the aforementioned methods, wherein the second material comprises a mesh; and the mesh comprises polypropylene or polyester.

In certain embodiments, the present invention relates to any one of the aforementioned methods, wherein the second material comprises a mesh; and the mesh comprises a biodegradable polymer.

In certain embodiments, the present invention relates to any one of the aforementioned methods, wherein the second material comprises a mesh; and the mesh comprises poly(glycolic acid), poly(lactic acid), or copolymers thereof.

### Use of a Polymerization Agent to Form a Hydrogel

In certain embodiments, the present invention relates to any one of the aforementioned methods, wherein the first material is a hydrogel; and the step of covering the defect, wound, or void with a first material comprises the steps of:

applying a first composition to the defect, wound or void; and

applying a second composition to the defect, wound or void,

wherein, after a first amount of time, application of the first composition and application of the second composition results in the formation of the first material.

In certain embodiments, the present invention relates to any one of the aforementioned methods, wherein the first material is a hydrogel; and the step of covering the defect, wound, or void with a first material comprises the step of:

applying a pre-hydrogel mixture to the defect, wound, or void;

wherein said pre-hydrogel mixture comprises a first composition and a second composition; and, after a first amount of time, the pre-hydrogel mixture gels, thereby forming the first material.

In certain embodiments, the present invention relates to any one of the aforementioned methods, further comprising the steps of:

combining a first composition and a second composition; and

waiting a second amount of time, thereby forming a pre-hydrogel mixture.

In certain embodiments, the present invention relates to any one of the aforementioned methods, wherein the second material is a hydrogel; and the step of covering the first material with a second material comprises the steps of:

applying a first composition over the first material; and

applying a second composition over the first material;

wherein, after a first amount of time, application of the first composition and the application of the second composition results in the formation of the second material.

In certain embodiments, the present invention relates to any one of the aforementioned methods, wherein the second material is a hydrogel; and the step of covering the first material with a second material comprises the step of:

applying a pre-hydrogel mixture over the first material;

wherein said pre-hydrogel mixture comprises a first composition and a second composition; and, after a first amount of time, the pre-hydrogel mixture gels, thereby forming the second material.

In certain embodiments, the present invention relates to any one of the aforementioned methods, further comprising the steps of:

combining a first composition and a second composition; and

waiting a second amount of time, thereby forming a pre-hydrogel mixture.

In certain embodiments, the present invention relates to any one of the aforementioned methods, wherein the first composition comprises a compound of formula Ia:

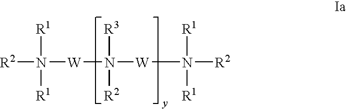

wherein, independently for each occurrence,

W is —CR₂—, —CR₂CR₂—, —CR₂CR₂CR₂—, —CR₂CR₂CR₂CR₂—, or —CR₂CR₂CR₂CR₂CR₂—;

R is hydrogen, methyl, ethyl, n-propyl, or i-propyl;

R¹ is hydrogen, alkyl, alkenyl, alkynyl,

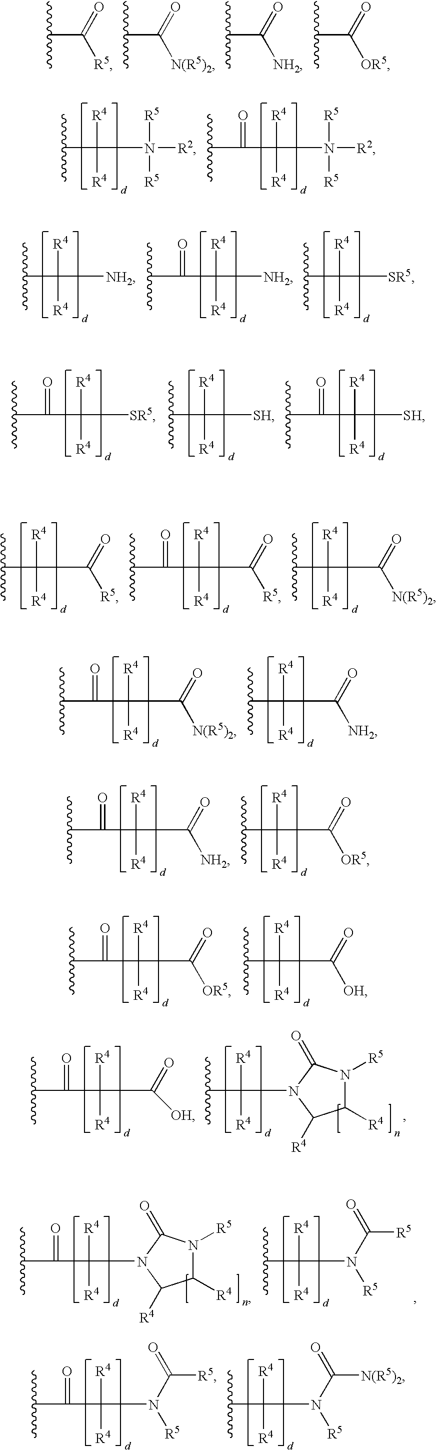

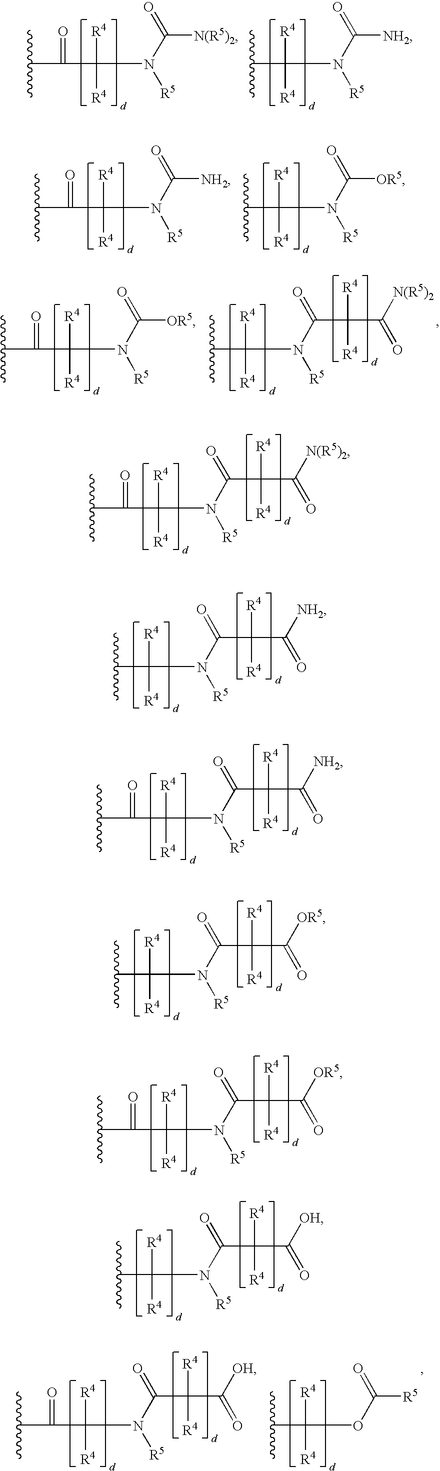

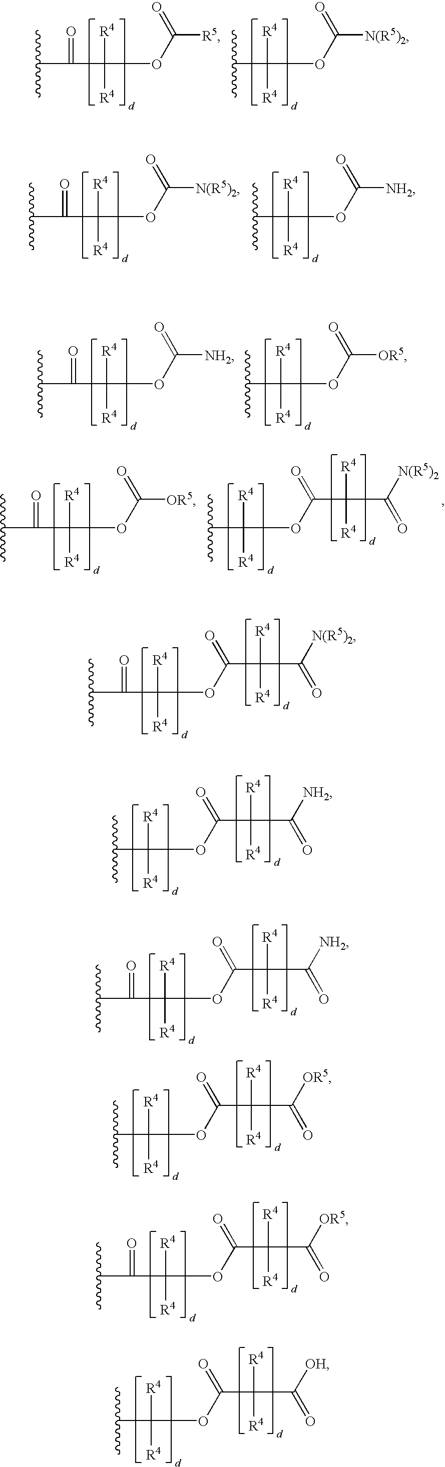

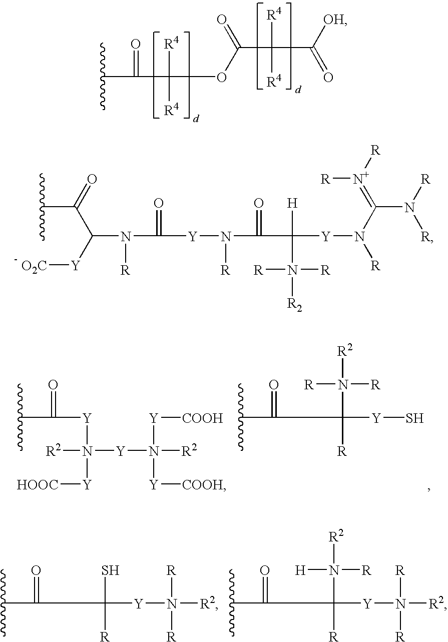

or a carbohydrate radical;

Y is —CR₂—, —CR₂CR₂—, —CR₂CR₂CR₂—, —CR₂CR₂CR₂CR₂—, or —CR₂CR₂CR₂CR₂CR₂—;

R² is an electron pair, hydrogen, alkyl, or aralkyl, provided that a pharmaceutically acceptable counter ion is present when R² is not an electron pair;

R³ is hydrogen, or

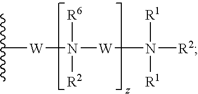

R⁴ is hydrogen, alkyl, alkoxyl, halogen, or aralkyl;

R⁵ is hydrogen, alkyl, aryl, or aralkyl;

R⁶ is hydrogen, or

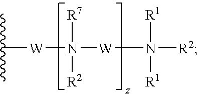

R⁷ is hydrogen, or

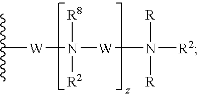

R⁸ is hydrogen, or

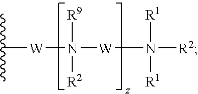

R⁹ is hydrogen, or

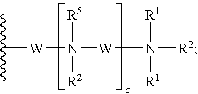

d is an integer from 1 to 10 inclusive;

n is an integer from 1 to 4 inclusive;

y is an integer from 5 to 40,000 inclusive; and

z is an integer from 0 to 20,000 inclusive;

provided that the sum of y and z is less than about 50,000.

In certain embodiments, the present invention relates to any one of the aforementioned methods, wherein at least about 5% of R¹ is hydrogen,

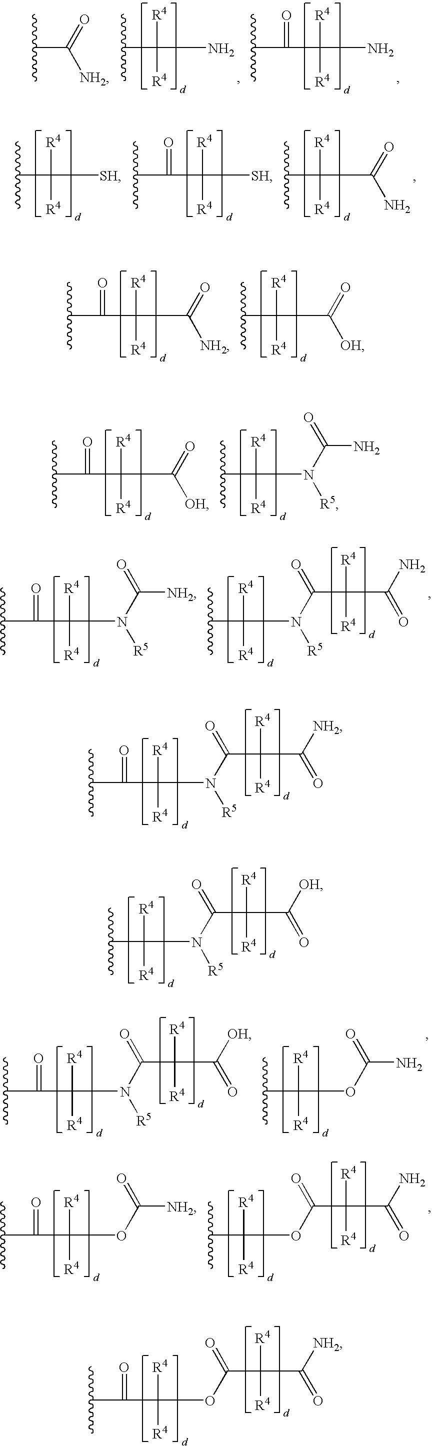

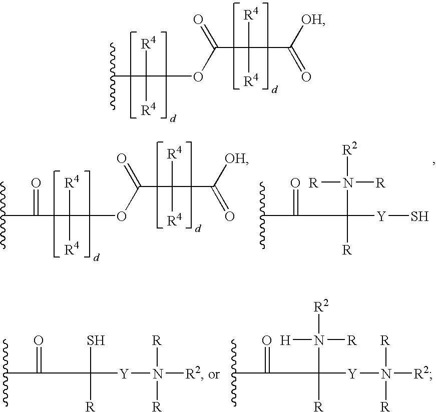

In certain embodiments, the present invention relates to any one of the aforementioned methods, wherein W is —CR₂CR₂—, —CR₂CR₂CR₂—, or —CR₂CR₂CR₂—.

In certain embodiments, the present invention relates to any one of the aforementioned methods, wherein W is —CH₂CH₂—, —CH₂CH₂CH₂—, or —CH₂CH₂CH₂—.

In certain embodiments, the present invention relates to any one of the aforementioned methods, wherein W is —CH₂CH₂—.

In certain embodiments, the present invention relates to any one of the aforementioned methods, wherein at least about 50% of R¹ is hydrogen.

In certain embodiments, the present invention relates to any one of the aforementioned methods, wherein at least about 70% of R¹ is hydrogen.

In certain embodiments, the present invention relates to any one of the aforementioned methods, wherein at least about 90% of R¹ is hydrogen.

In certain embodiments, the present invention relates to any one of the aforementioned methods, wherein at least about 95% of R¹ is hydrogen.

In certain embodiments, the present invention relates to any one of the aforementioned methods, wherein R¹ is

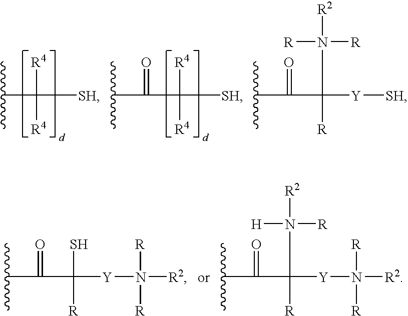

In certain embodiments, the present invention relates to any one of the aforementioned methods, wherein R¹ is

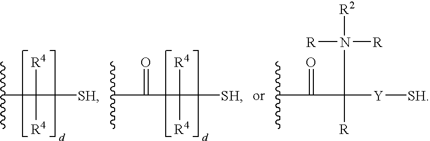

In certain embodiments, the present invention relates to any one of the aforementioned methods, wherein R² is hydrogen.

In certain embodiments, the present invention relates to any one of the aforementioned methods, wherein R² is an electron pair.

In certain embodiments, the present invention relates to any one of the aforementioned methods, wherein R⁶ is hydrogen.

In certain embodiments, the present invention relates to any one of the aforementioned methods, wherein R⁷ is hydrogen.

In certain embodiments, the present invention relates to any one of the aforementioned methods, wherein R⁸ is hydrogen.

In certain embodiments, the present invention relates to any one of the aforementioned methods, wherein R⁹ is hydrogen.

In certain embodiments, the present invention relates to any one of the aforementioned methods, wherein d is 1-8 inclusive.

In certain embodiments, the present invention relates to any one of the aforementioned methods, wherein n is 1 or 2.

In certain embodiments, the present invention relates to any one of the aforementioned methods, wherein the sum or y and z is an integer from about 50 to about 200.

In certain embodiments, the present invention relates to any one of the aforementioned methods, wherein said compound of formula Ia is NH₂(CH₂)₂N(H)(CH₂)₄N(H)(CH₂)₂NH₂ or NH₂(CH₂)₃N(H)(CH₂)₄N(H)(CH₂)₃NH₂.

In certain embodiments, the present invention relates to any one of the aforementioned methods, wherein said compound of formula Ia has a weight average molecular weight of about 600 Daltons to about 10,000 Daltons.

In certain embodiments, the present invention relates to any one of the aforementioned methods, wherein the first composition comprises a compound of formula Ib selected from the group consisting of

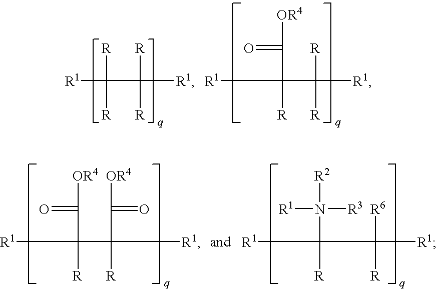

wherein, independently for each occurrence,

R is hydrogen, alkyl, aryl, or aralkyl;

R¹ is hydrogen, alkyl, or a polymerization inhibitor;

R² is an electron pair, hydrogen, alkyl, or aralkyl, provided that a pharmaceutically acceptable counter ion is present when R² is not an electron pair;

R³ is hydrogen, alkyl, aryl, aralkyl, acyl,

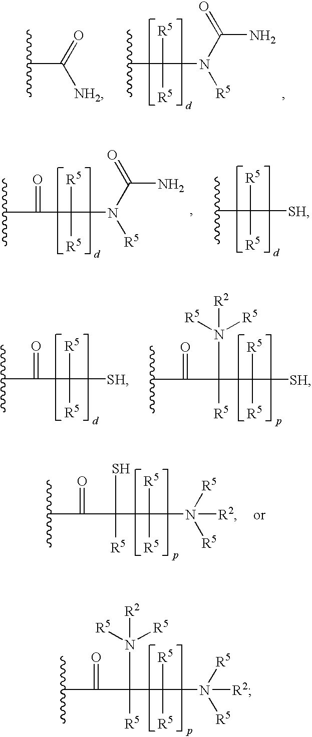

R⁴ is hydrogen, alkyl, aryl, aralkyl,

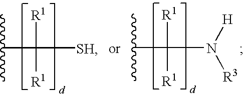

R⁵ is hydrogen, or alkyl;

R⁶ is hydrogen, alkyl, aryl, —C(O)OR⁴, or —OC(O)R⁴;

d is an integer from 1 to 8 inclusive;

p is an integer from 1 to 5 inclusive; and

q is an integer from 50 to 100,000 inclusive.

In certain embodiments, the present invention relates to any one of the aforementioned methods, wherein the compound of formula Ib is

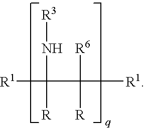

In certain embodiments, the present invention relates to any one of the aforementioned methods, wherein the compound of formula Ib is

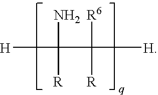

In certain embodiments, the present invention relates to any one of the aforementioned methods, wherein the compound of formula Ib is

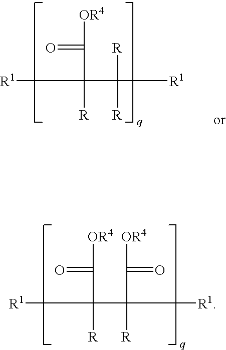

In certain embodiments, the present invention relates to any one of the aforementioned methods, wherein the compound of formula Ib is

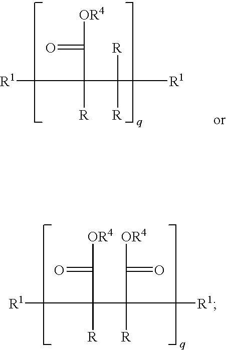

and R⁴ is —(CH₂)_{d}NH₂.

In certain embodiments, the present invention relates to any one of the aforementioned methods, wherein the first composition comprises a polymer having one or more monomeric units represented by formula Ie:

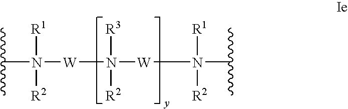

wherein, independently for each occurrence,

W is —CR₂—, —CR₂CR₂—, —CR₂CR₂CR₂—, —CR₂CR₂CR₂CR₂—, or —CR₂CR₂CR₂CR₂CR₂—;

R is hydrogen, methyl, ethyl, n-propyl, or i-propyl;

R¹ is hydrogen, alkyl, alkenyl, alkynyl,

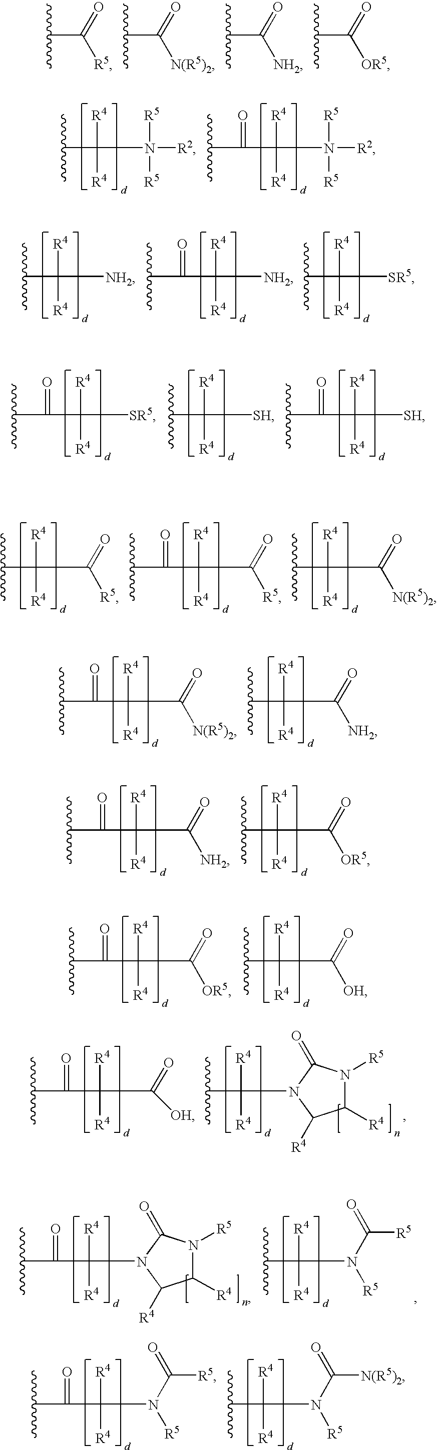

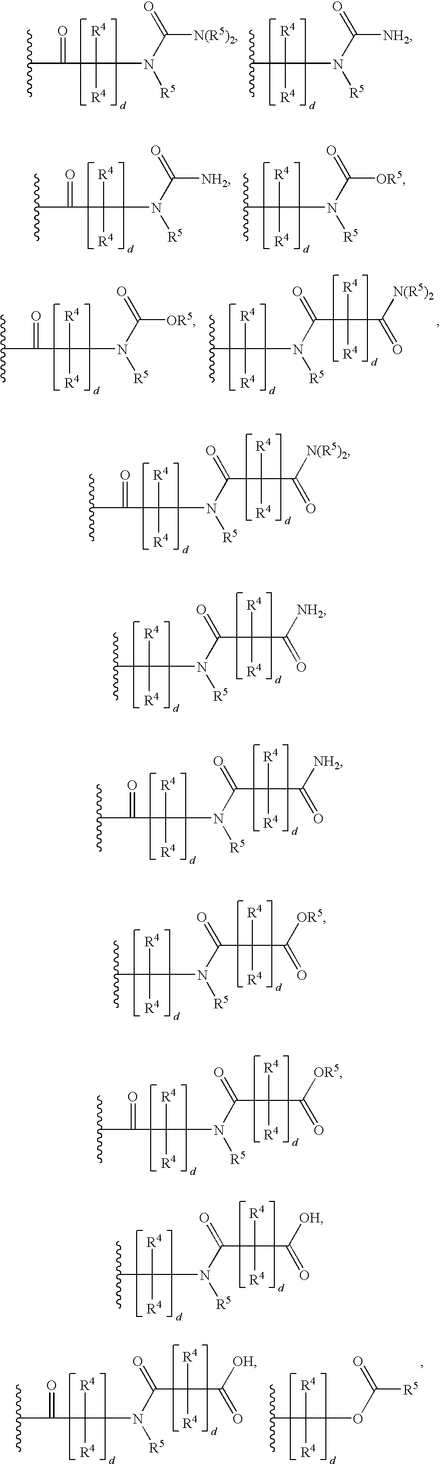

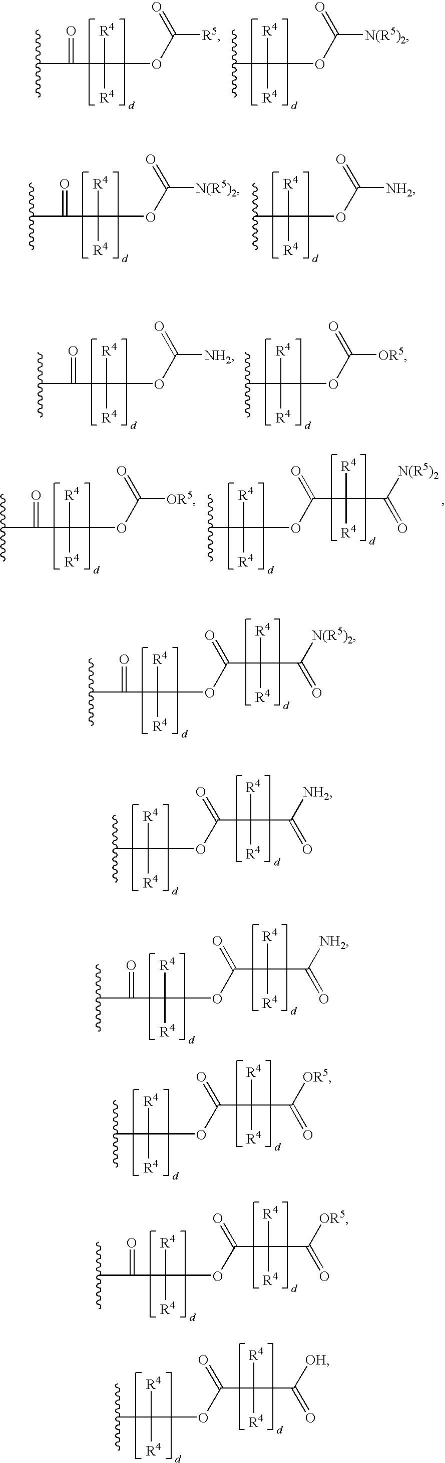

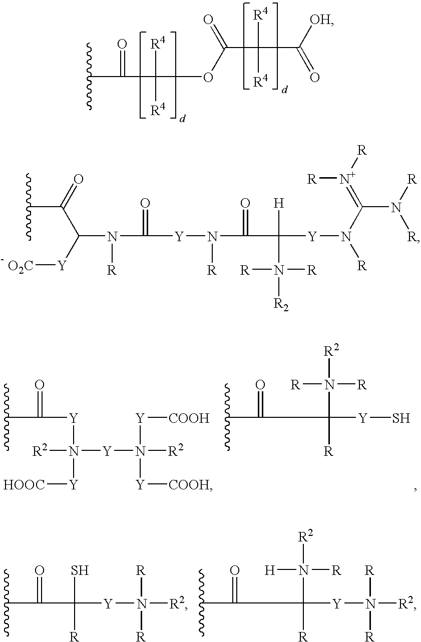

or a carbohydrate radical;

Y is —CR₂—, —CR₂CR₂—, —CR₂CR₂CR₂—, —CR₂CR₂CR₂CR₂—, or —CR₂CR₂CR₂CR₂CR₂—;

R² is an electron pair, hydrogen, alkyl, or aralkyl, provided that a pharmaceutically acceptable counter ion is present when R² is not an electron pair;

R³ is hydrogen, or

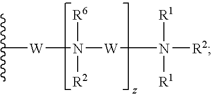

R⁴ is hydrogen, alkyl, alkoxyl, halogen, or aralkyl;

R⁵ is hydrogen, alkyl, aryl, or aralkyl;

R⁶ is hydrogen, or

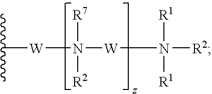

R⁷ is hydrogen, or

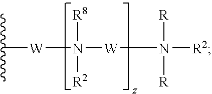

R⁸ is hydrogen, or

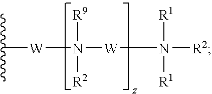

R⁹ is hydrogen, or

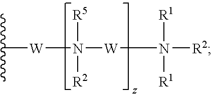

d is an integer from 1 to 10 inclusive;

n is an integer from 1 to 4 inclusive;

y is an integer from 5 to 40,000 inclusive; and

z is an integer from 0 to 20,000 inclusive;

provided that the sum of y and z is less than about 50,000.

In certain embodiments, the present invention relates to any one of the aforementioned methods, wherein at least about 5% of R¹ is hydrogen,

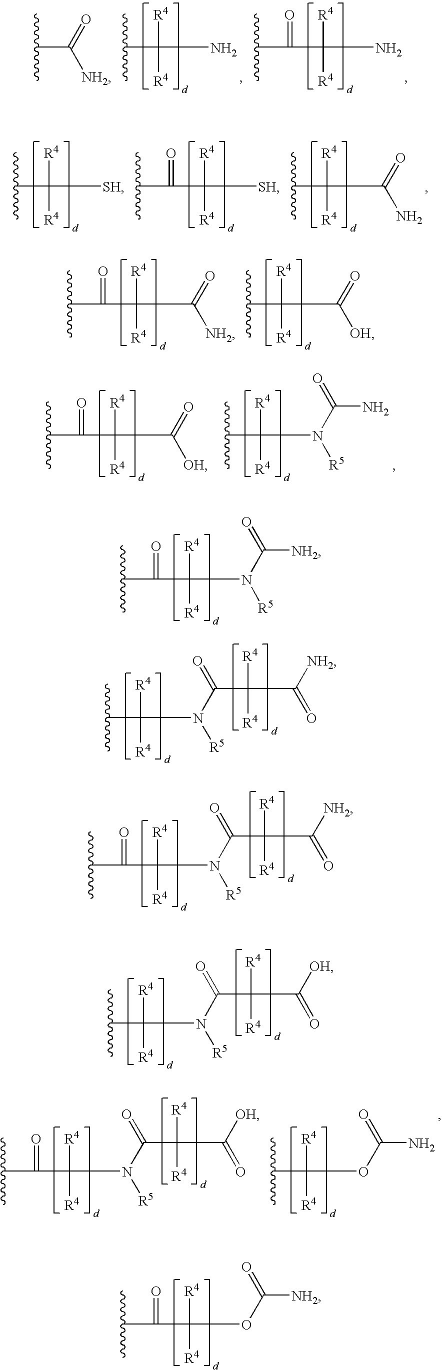

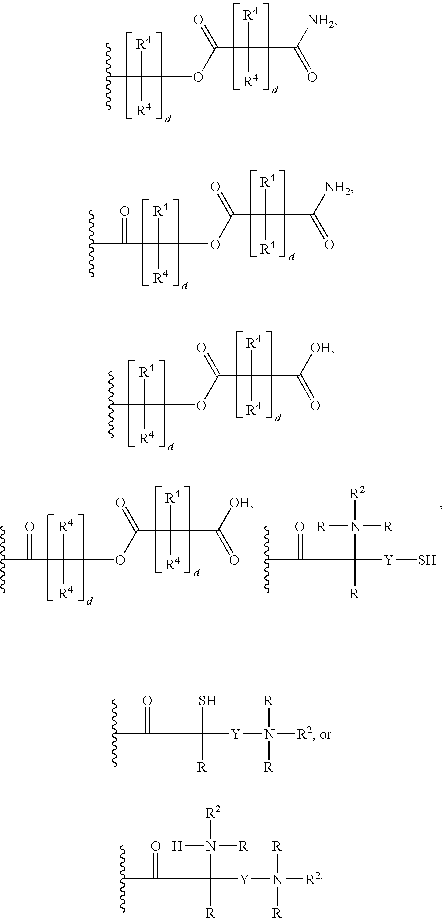

In certain embodiments, the present invention relates to any one of the aforementioned methods, wherein W is —CR₂CR₂—, —CR₂CR₂CR₂—, or —CR₂CR₂CR₂—.

In certain embodiments, the present invention relates to any one of the aforementioned methods, W is —CH₂CH₂—, —CH₂CH₂CH₂—, or —CH₂CH₂CH₂—.

In certain embodiments, the present invention relates to any one of the aforementioned methods, wherein at least about 50% of R¹ is hydrogen.

In certain embodiments, the present invention relates to any one of the aforementioned methods, wherein at least about 70% of R¹ is hydrogen.

In certain embodiments, the present invention relates to any one of the aforementioned methods, wherein at least about 90% of R¹ is hydrogen.

In certain embodiments, the present invention relates to any one of the aforementioned methods, wherein at least about 95% of R¹ is hydrogen.

In certain embodiments, the present invention relates to any one of the aforementioned methods, wherein R¹ is

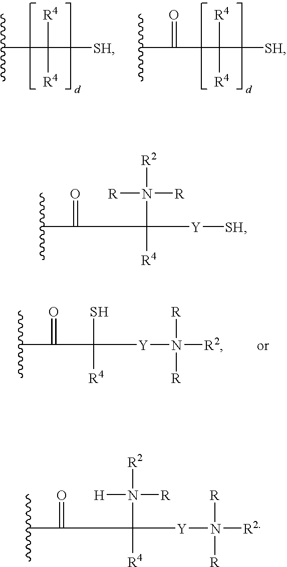

In certain embodiments, the present invention relates to any one of the aforementioned methods, wherein R¹ is

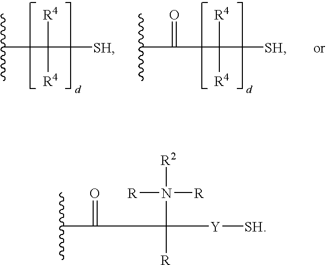

In certain embodiments, the present invention relates to any one of the aforementioned methods, wherein R is hydrogen.

In certain embodiments, the present invention relates to any one of the aforementioned methods, wherein R² is an electron pair.

In certain embodiments, the present invention relates to any one of the aforementioned methods, wherein R⁶ is hydrogen.

In certain embodiments, the present invention relates to any one of the aforementioned methods, wherein R⁷ is hydrogen.

In certain embodiments, the present invention relates to any one of the aforementioned methods, wherein R⁸ is hydrogen.

In certain embodiments, the present invention relates to any one of the aforementioned methods, wherein R⁹ is hydrogen.

In certain embodiments, the present invention relates to any one of the aforementioned methods, wherein d is 1-8 inclusive.

In certain embodiments, the present invention relates to any one of the aforementioned methods, wherein n is 1 or 2.

In certain embodiments, the present invention relates to any one of the aforementioned methods, wherein the sum or y and z is an integer from about 50 to about 200.

In certain embodiments, the present invention relates to any one of the aforementioned methods, wherein the first composition further comprises a compound of formula IV selected from the group consisting of

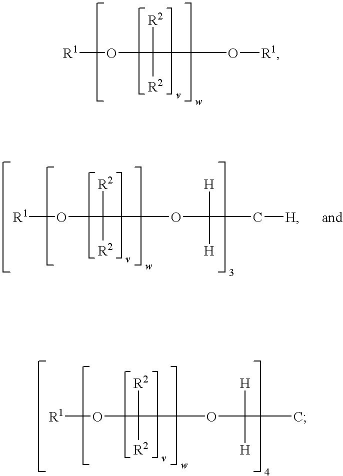

wherein, independently for each occurrence,

R¹ is

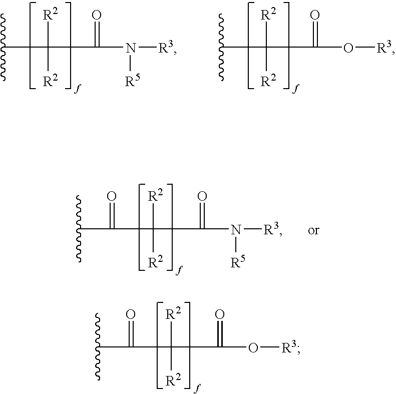

R² is hydrogen, alkyl, or halogen;

R³ is

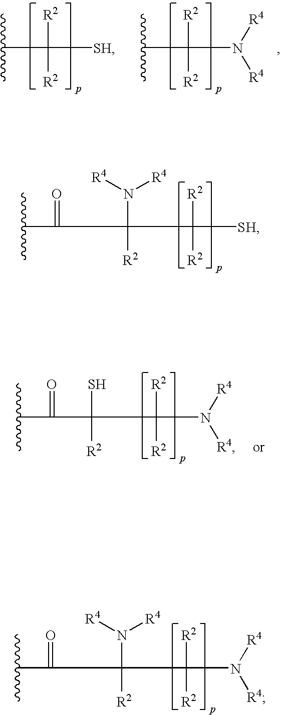

R⁴ is hydrogen, alkyl, aryl, aralkyl,

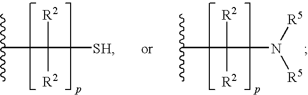

R⁵ is hydrogen, alkyl, or aralkyl;

f is an integer from 1 to 25 inclusive;

k is an integer from 1 to 25 inclusive;

p is an integer from 1 to 5 inclusive;

v is an integer from 2 to 4 inclusive; and

w is an integer from 5 to 1,000 inclusive.

In certain embodiments, the present invention relates to any one of the aforementioned methods, wherein the second composition comprises a compound of formula III selected from the group consisting of R¹-(alkyl diradical)-R¹, R¹-(heteroalkyl diradical)-R¹,

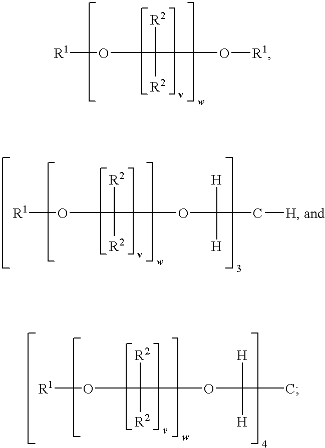

wherein, independently for each occurrence,

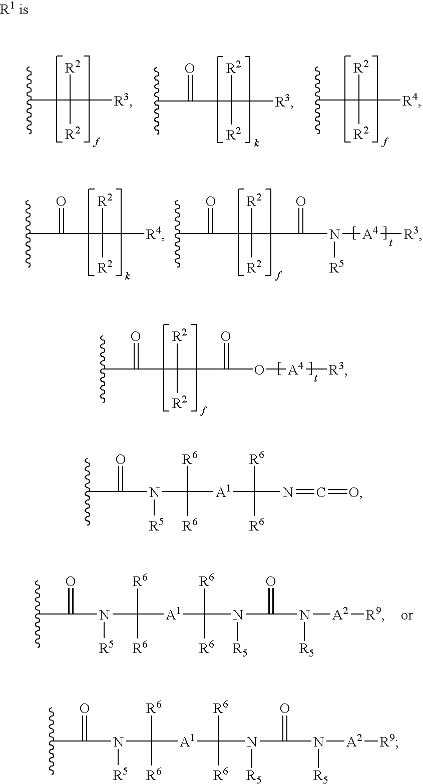

R² is hydrogen, alkyl, or halogen;

R³ is —C(O)H, —C(O)alkyl, —C(O)fluoroalkyl, —C(O)chloroalkyl, —C(O)CH₂NO₂,

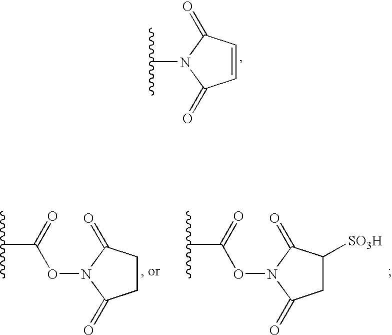

R⁴ is —N═C═O, —N═C═S,

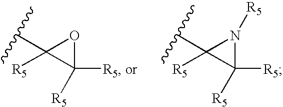

R⁵ is hydrogen, alkyl, or aralkyl;

R⁶ is hydrogen, or C₁-C₆ alkyl;

R⁷ is —CO₂H, —(CR⁶₂)ₚN═C═O,

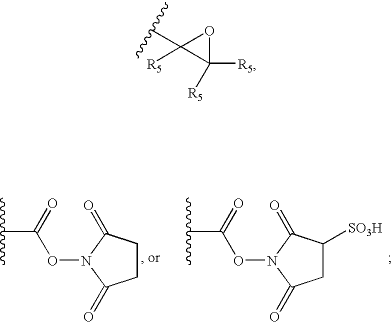

R⁸ is

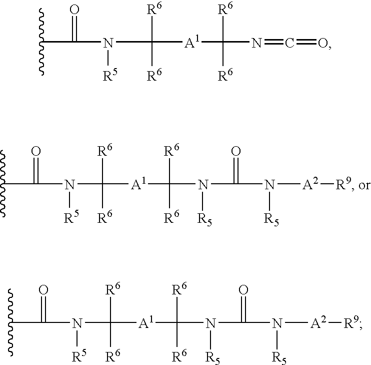

R⁹ is

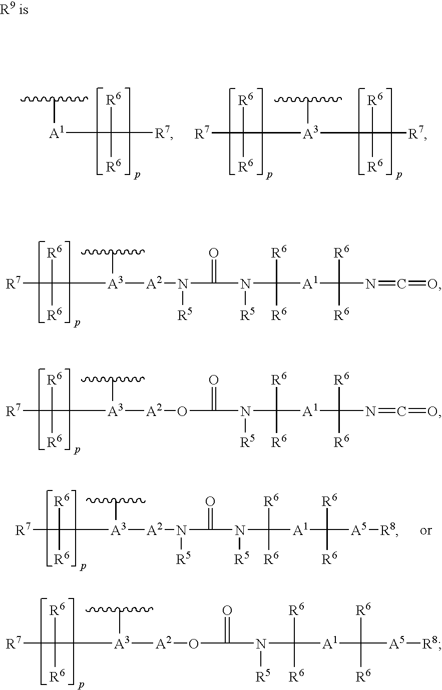

A¹ is an alkyl diradical, a heteroalkyl diradical, a cycloalkyl diradical, a heterocycloalkyl diradical, an alkenyl diradical, an alkynyl diradical, an aryl diradical, a heteroaryl diradical, an aralkyl diradical, or a heteroaralkyl diradical;

A² is a bond, an alkyl diradical, a heteroalkyl diradical, a cycloalkyl diradical, a heterocycloalkyl diradical, an alkenyl diradical, an alkynyl diradical, an aryl diradical, a heteroaryl diradical, an aralkyl diradical, or a heteroaralkyl diradical;

A³ is an alkyl triradical, a heteroalkyl triradical, a cycloalkyl triradical, a heterocycloalkyl triradical, an alkenyl triradical, an aryl triradical, a heteroaryl triradical, an aralkyl triradical, or a heteroaralkyl triradical;

A⁴ is an alkyl diradical, a cycloalkyl diradical, an aryl diradical, or anaralkyl diradical;

A⁵ is an alkyl diradical, a heteroalkyl diradical, or

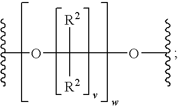

f is an integer from 1 to 25 inclusive;

k is an integer from 1 to 25 inclusive;

p is an integer from 0 to 5 inclusive;

t is an integer from 1 to 4 inclusive;

v is an integer from 2 to 4 inclusive; and

w is an integer from 5 to 1,000 inclusive.

In certain embodiments, the present invention relates to any one of the aforementioned methods, wherein w is an integer in the range of about 50 to about 250.

In certain embodiments, the present invention relates to any one of the aforementioned methods, wherein w is an integer in the range of about 60 to about 90.

In certain embodiments, the present invention relates to any one of the aforementioned methods, wherein w is an integer in the range of about 15 to about 90.

In certain embodiments, the present invention relates to any one of the aforementioned methods, wherein the compound of formula III is

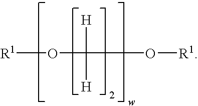

In certain embodiments, the present invention relates to any one of the aforementioned methods, wherein R¹ is

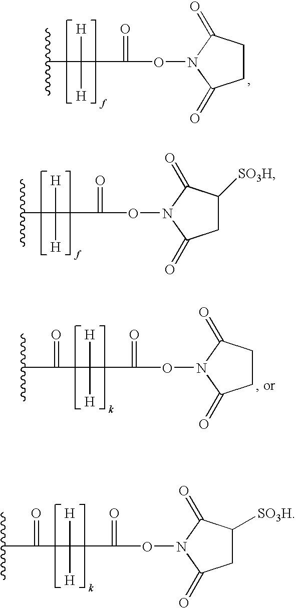

In certain embodiments, the present invention relates to any one of the aforementioned methods, wherein the compound of formula III is

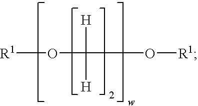

### and R¹ is

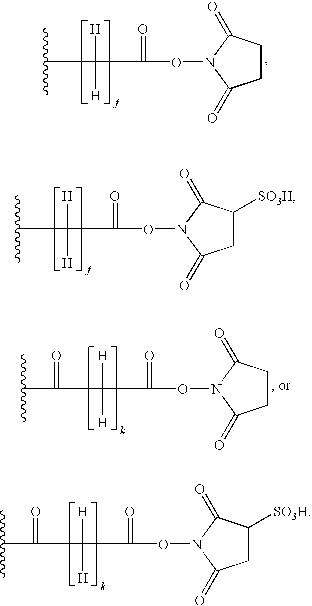

In certain embodiments, the present invention relates to any one of the aforementioned methods, wherein the compound of formula III is

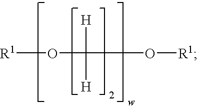

### R¹ is

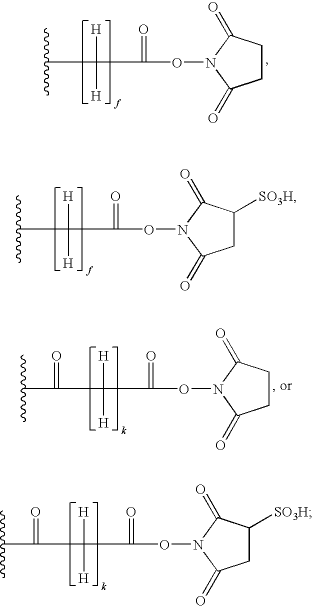

and w is an integer in the range of about 15 to about 90.

In certain embodiments, the present invention relates to any one of the aforementioned methods, wherein the compound of formula III is

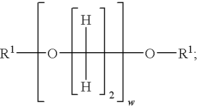

### R¹ is

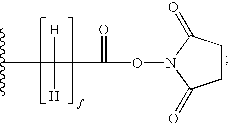

and f is 3.

In certain embodiments, the present invention relates to any one of the aforementioned methods, wherein the compound of formula III is

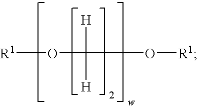

### R¹ is

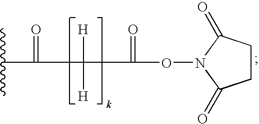

and k is 2, or 3.

In certain embodiments, the present invention relates to any one of the aforementioned methods, wherein the compound of formula III is

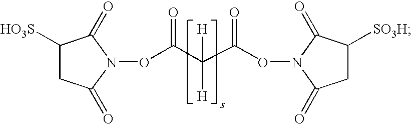

and s is an integer in the range of 1 to 20 inclusive.

In certain embodiments, the present invention relates to any one of the aforementioned methods, wherein R⁹ is

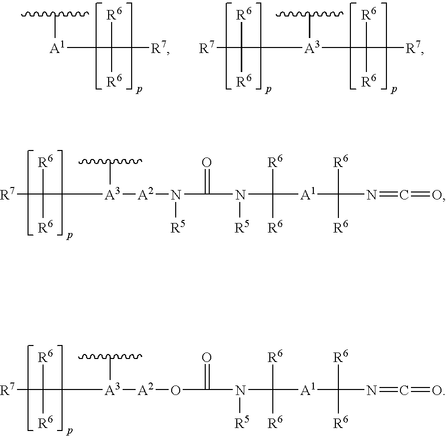

In certain embodiments, the present invention relates to any one of the aforementioned methods, wherein the compound of formula III is

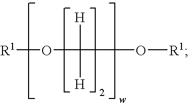

### A⁵ is

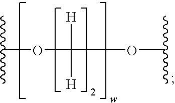

and A¹ is an aryl diradical.

In certain embodiments, the present invention relates to any one of the aforementioned methods, wherein the compound of formula III is

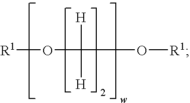

### A⁵ is

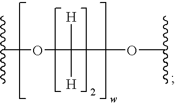

and A¹ is an optionally substituted phenyl diradical.

In certain embodiments, the present invention relates to any one of the aforementioned methods, wherein the compound of formula III is

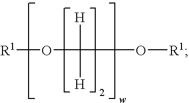

### A⁵ is

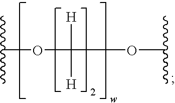

A² is a bond; and A¹ is an alkyl diradical.

In certain embodiments, the present invention relates to any one of the aforementioned methods, wherein the compound of formula III is

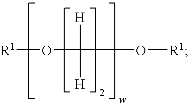

### A⁵ is

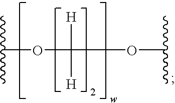

A² is a bond; and A³ is an alkyl triradical.

In certain embodiments, the present invention relates to any one of the aforementioned methods, wherein the compound of formula III is

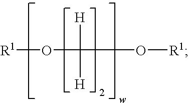

### A⁵ is

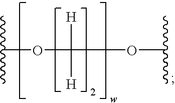

A² is a bond; A¹ is an alkyl diradical; and R⁷ is

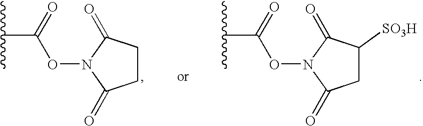

In certain embodiments, the present invention relates to any one of the aforementioned methods, wherein the compound of formula III is

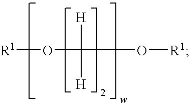

### A⁵ is

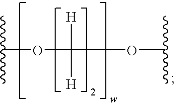

A² is a bond; A³ is an alkyl triradical; and R⁷ is

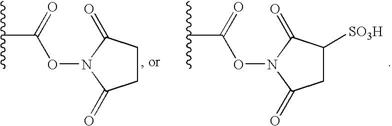

In certain embodiments, the present invention relates to any one of the aforementioned methods, wherein the compound of formula III is

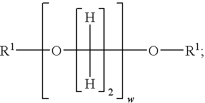

### A⁵ is

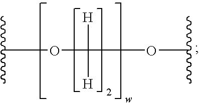

A² is an aryl diradical; A¹ is an aralkyl diradical; and R⁷ is

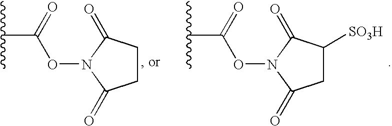

In certain embodiments, the present invention relates to any one of the aforementioned methods, wherein the compound of formula III is

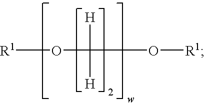

### A⁵ is

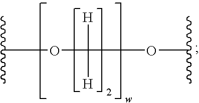

A² is an aryl diradical; A³ is an aralkyl triradical; and R⁷ is

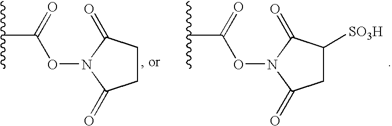

In certain embodiments, the present invention relates to any one of the aforementioned methods, wherein the compound of formula III is

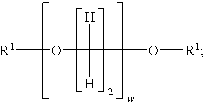

### A⁵ is

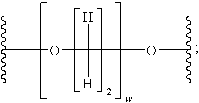

A² is an optionally substituted phenyl diradical; A¹ is an optionally substituted benzyl diradical; and R⁷ is

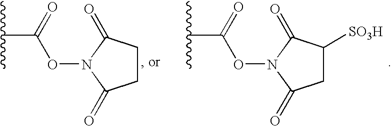

In certain embodiments, the present invention relates to any one of the aforementioned methods, wherein the compound of formula III is

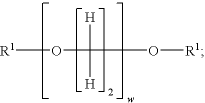

### A⁵ is

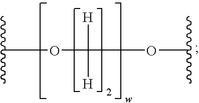

A² is an optionally substituted phenyl diradical; A³ is an optionally substituted benzyl triradical; and R⁷ is

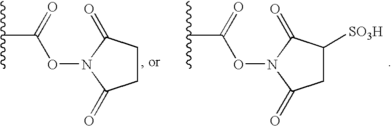

In certain embodiments, the present invention relates to any one of the aforementioned methods, wherein the compound of formula III is

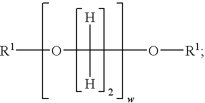

### and R¹ is

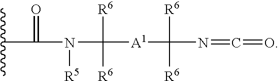

In certain embodiments, the present invention relates to any one of the aforementioned methods, wherein the compound of formula III is

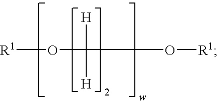

### R¹ is

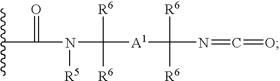

R⁶ is C₁-C₆ alkyl; and A¹ is an optionally substituted phenyl diradical.

In certain embodiments, the present invention relates to any one of the aforementioned methods, wherein the compound of formula III is

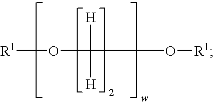

### R¹ is

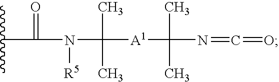

and A¹ is a phenyl diradical.

In certain embodiments, the present invention relates to any one of the aforementioned methods, wherein R¹ is —CH₂C(O)N(H)-A⁴-R³; A⁴ is an alkyl diradical; and R³ is

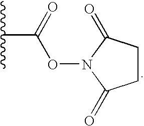

In certain embodiments, the present invention relates to any one of the aforementioned methods, wherein f is an integer from 1 to 9 inclusive.

In certain embodiments, the present invention relates to any one of the aforementioned methods, wherein k is an integer from 1 to 9 inclusive.

In certain embodiments, the present invention relates to any one of the aforementioned methods, wherein said compound of formula III has a weight average molecular weight of about 500 Daltons to about 20,000 Daltons.

In certain embodiments, the present invention relates to any one of the aforementioned methods, wherein the compound of formula III is

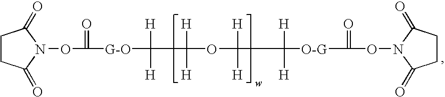

wherein, independently for each occurrence,

G is —CR₂CR₂—, —CR₂CR₂CR₂—, —CR₂CR₂CR₂CR₂—, —CR₂CR₂CR₂CR₂CR₂—, —CR₂CR₂CR₂CR₂CR₂CR₂—, —CR₂CR₂CR₂CR₂CR₂CR₂CR₂—, —CR₂CR₂CR₂CR₂CR₂CR₂CR₂CR₂—, —CR₂CR₂CR₂CR₂CR₂CR₂CR₂CR₂CR₂—, or —CR₂CR₂CR₂CR₂CR₂CR₂CR₂CR₂CR₂—; and

R is hydrogen or methyl.

In certain embodiments, the present invention relates to any one of the aforementioned methods, wherein G is —CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, —CH₂CH₂CH₂CH₂CH₂CH₂CH₂CH₂—, —CH₂CH(CH₃)CH₂—, —CH₂C(CH₃)₂CH₂—, or —C(CH₃)₂CH₂CH₂—.

In certain embodiments, the present invention relates to any one of the aforementioned methods, wherein the second composition further comprises a compound of formula IV selected from the group consisting of

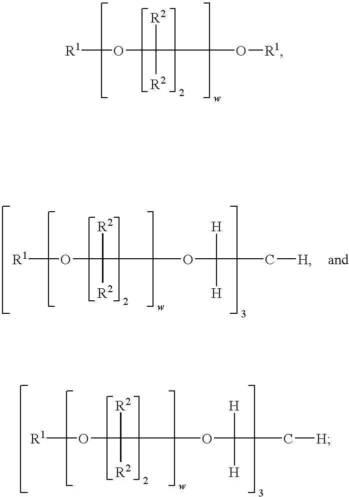

wherein, independently for each occurrence,

R¹ is

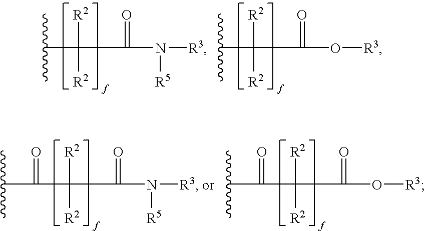

R² is hydrogen, alkyl, or halogen;

R³ is

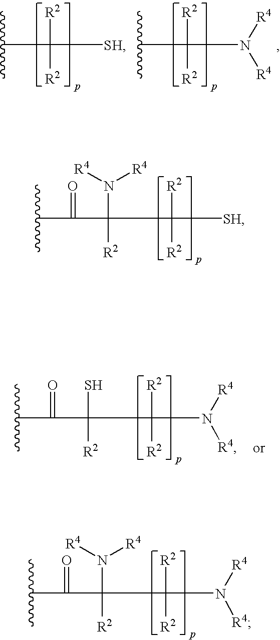

R⁴ is hydrogen, alkyl, aryl, aralkyl,

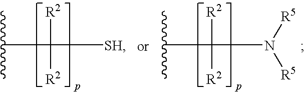

R⁵ is hydrogen, or alkyl;

f is an integer from 1 to 25 inclusive;

k is an integer from 1 to 25 inclusive;

p is an integer from 1 to 5 inclusive;

v is an integer from 2 to 4 inclusive; and

w is an integer from 5 to 1,000 inclusive.

In certain embodiments, the present invention relates to any one of the aforementioned methods, wherein the first composition comprises a compound of formula Ia:

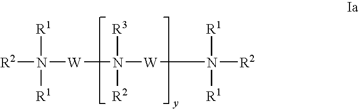

wherein, independently for each occurrence,

W is —CR₂—, —CR₂CR₂—, —CR₂CR₂CR₂—, —CR₂CR₂CR₂CR₂—, or —CR₂CR₂CR₂CR₂CR₂—;

R¹ is hydrogen, alkyl, alkenyl, alkynyl,

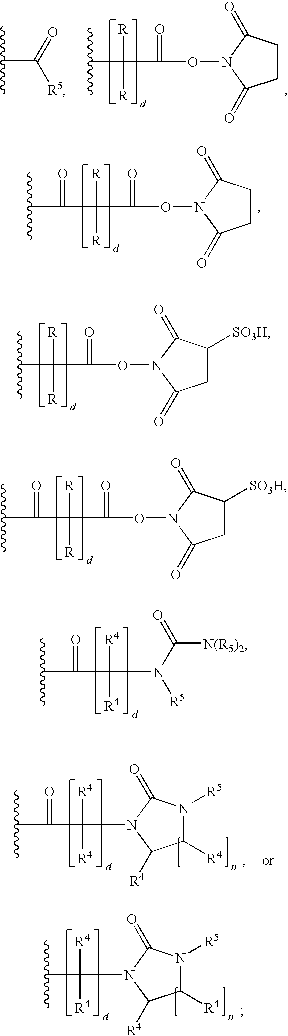

R² is an electron pair, hydrogen, alkyl, or aralkyl, provided that a pharmaceutically acceptable counter ion is present when R² is not an electron pair;

R³ is hydrogen, or

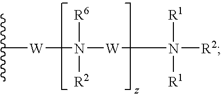

R⁴ is hydrogen, alkyl, alkoxyl, halogen, or aralkyl;

R⁵ is hydrogen, alkyl, aryl, or aralkyl;

R⁶ is hydrogen, or

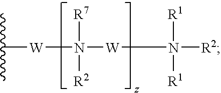

R⁷ is hydrogen, or

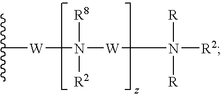

R⁸ is hydrogen, or

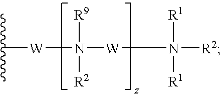

R⁹ is hydrogen, or

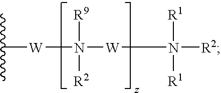

d is an integer from 1 to 8 inclusive;

n is an integer from 1 to 4 inclusive;

y is an integer from 5 to 40,000 inclusive; and

z is an integer from 0 to 20,000 inclusive;

provided that the sum of y and z is less than about 50,000.

In certain embodiments, the present invention relates to any one of the aforementioned methods, wherein 5% of R¹ is

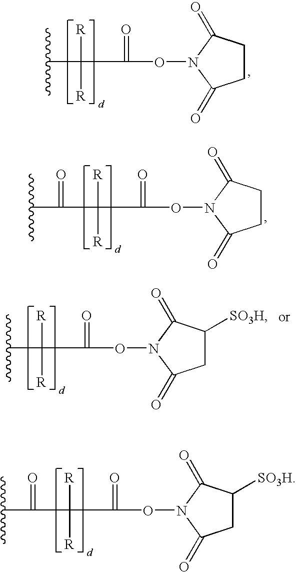

In certain embodiments, the present invention relates to any one of the aforementioned methods, wherein at least about 10% of R¹ is

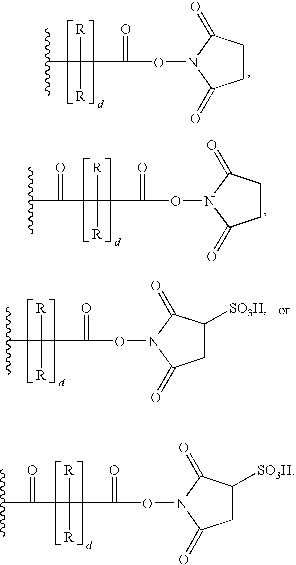

In certain embodiments, the present invention relates to any one of the aforementioned methods, wherein at least about 25% of R¹ is

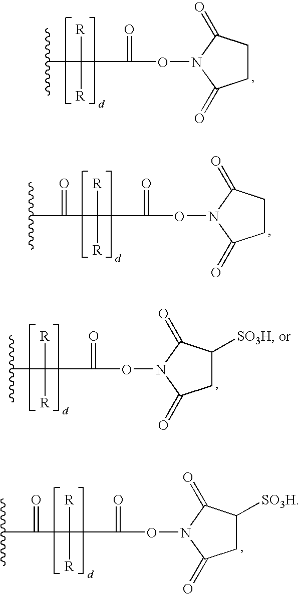

In certain embodiments, the present invention relates to any one of the aforementioned methods, wherein at least about 50% R¹ is

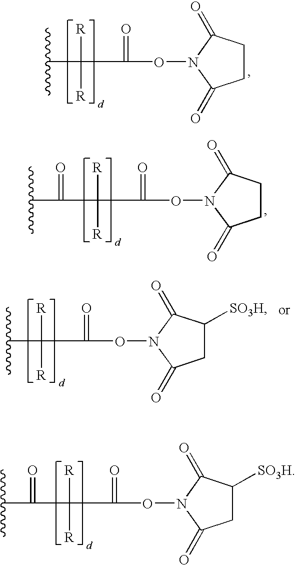

In certain embodiments, the present invention relates to any one of the aforementioned methods, wherein R is hydrogen.

In certain embodiments, the present invention relates to any one of the aforementioned methods, wherein W is —CR₂CR₂—, —CR₂CR₂CR₂—, or —CR₂CR₂CR₂—.

In certain embodiments, the present invention relates to any one of the aforementioned methods, wherein W is —CH₂CH₂—, —CH₂CH₂CH₂—, or —CH₂CH₂CH₂—.

In certain embodiments, the present invention relates to any one of the aforementioned methods, wherein W is —CH₂CH₂—.

In certain embodiments, the present invention relates to any one of the aforementioned methods, wherein R² is an electron pair.

In certain embodiments, the present invention relates to any one of the aforementioned methods, wherein R⁶ is hydrogen.

In certain embodiments, the present invention relates to any one of the aforementioned methods, wherein R⁷ is hydrogen.

In certain embodiments, the present invention relates to any one of the aforementioned methods, wherein R⁸ is hydrogen.

In certain embodiments, the present invention relates to any one of the aforementioned methods, wherein R⁹ is hydrogen.

In certain embodiments, the present invention relates to any one of the aforementioned methods, wherein d is 1-8 inclusive.

In certain embodiments, the present invention relates to any one of the aforementioned methods, wherein n is 1 or 2.

In certain embodiments, the present invention relates to any one of the aforementioned methods, wherein the sum or y and z is an integer from about 50 to about 200.

In certain embodiments, the present invention relates to any one of the aforementioned methods, wherein the first composition comprises a compound of formula Ib selected from the

group consisting of

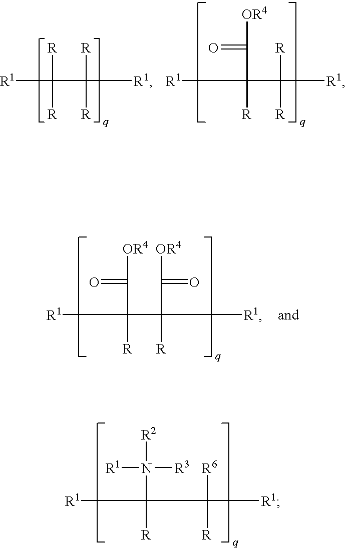

wherein, independently for each occurrence,

R¹ is hydrogen, alkyl, aryl, or aralkyl;

R¹ is hydrogen, alkyl,

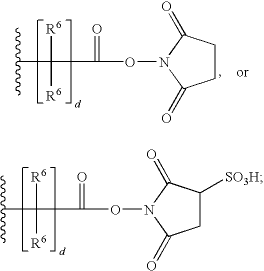

R² is an electron pair, hydrogen, alkyl, or aralkyl, provided that a pharmaceutically acceptable counter ion is present when R² is not an electron pair;

R³ is hydrogen, alkyl, aryl, aralkyl, acyl,

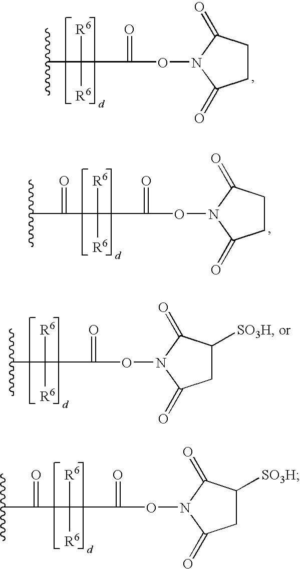

R⁴ is hydrogen, alkyl, aryl, aralkyl,

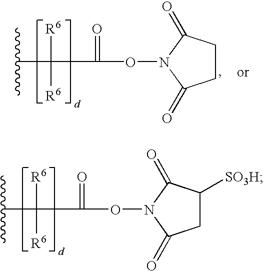

R⁵ is hydrogen, or alkyl;

R⁶ is hydrogen, or C₁-C₃ alkyl;

d is an integer from 1 to 8 inclusive;

p is an integer from 1 to 5 inclusive; and

q is an integer from 50 to 100,000 inclusive.

In certain embodiments, the present invention relates to any one of the aforementioned methods, wherein the compound of formula Ib is

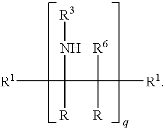

In certain embodiments, the present invention relates to any one of the aforementioned methods, wherein the compound of formula Ib is

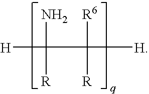

In certain embodiments, the present invention relates to any one of the aforementioned methods, wherein the compound of formula Ib is

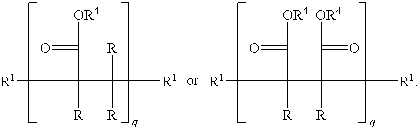

In certain embodiments, the present invention relates to any one of the aforementioned methods, wherein R¹ is

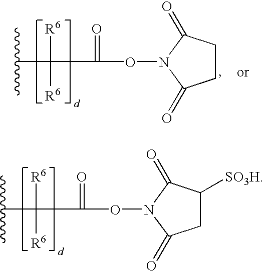

In certain embodiments, the present invention relates to any one of the aforementioned methods, wherein R³ is

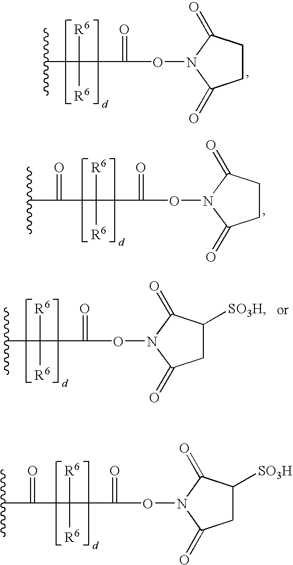

In certain embodiments, the present invention relates to any one of the aforementioned methods, wherein R⁴ is

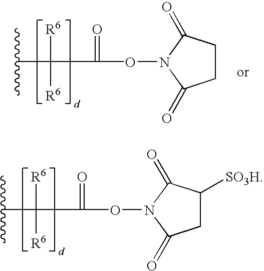

In certain embodiments, the present invention relates to any one of the aforementioned methods, wherein the first composition comprises a compound of formula Ic:

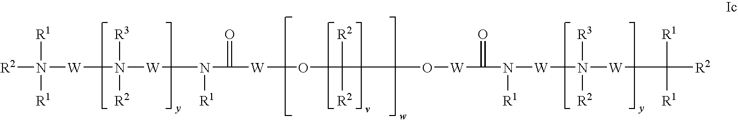

wherein, independently for each occurrence,

W is —CR₂—, —CR₂CR₂—, —CR₂CR₂CR₂—, —CR₂CR₂CR₂CR₂—, or —CR₂CR₂CR₂CR₂CR₂—;

R is hydrogen, methyl, ethyl, n-propyl, or i-propyl;

R¹ is hydrogen, alkyl, alkenyl, alkynyl,

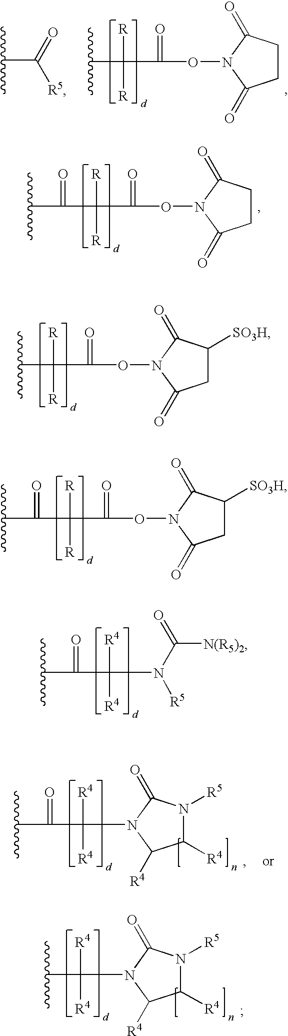

R² is an electron pair, hydrogen, alkyl, or aralkyl, provided that a pharmaceutically acceptable counter ion is present when R² is not an electron pair;

R³ is hydrogen, or

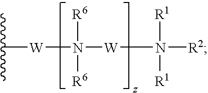

R⁴ is hydrogen, alkyl, alkoxyl, halogen, or aralkyl;

R⁵ is hydrogen, alkyl, aryl, or aralkyl;

R⁶ is hydrogen, or

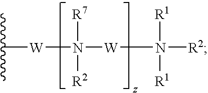

R⁷ is hydrogen, or

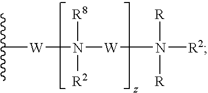

R⁸ is hydrogen, or

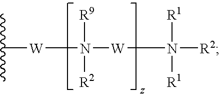

R⁹ is hydrogen, or

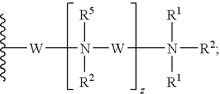

d is an integer from 1 to 8 inclusive;

n is an integer from 1 to 4 inclusive;

p is an integer from 1 to 5 inclusive; and

v is an integer from 2 to 4 inclusive;

w is an integer from 5 to 1,000;

y is an integer from 5 to 40,000 inclusive; and

z is an integer from 0 to 20,000 inclusive;

provided that the sum of y and z is less than about 50,000.

In certain embodiments, the present invention relates to any one of the aforementioned methods, wherein R is hydrogen.

In certain embodiments, the present invention relates to any one of the aforementioned methods, wherein W is —CR₂CR₂—, —CR₂CR₂CR₂—, or —CR₂CR₂CR₂—.

In certain embodiments, the present invention relates to any one of the aforementioned methods, wherein W is —CH₂CH₂—, —CH₂CH₂CH₂—, or —CH₂CH₂CH₂—.

In certain embodiments, the present invention relates to any one of the aforementioned methods, wherein at least about 50% of R¹ is hydrogen.

In certain embodiments, the present invention relates to any one of the aforementioned methods, wherein at least about 70% of R¹ is hydrogen.

In certain embodiments, the present invention relates to any one of the aforementioned methods, wherein at least about 90% of R¹ is hydrogen.

In certain embodiments, the present invention relates to any one of the aforementioned methods, wherein at least about 95% of R¹ is hydrogen.

In certain embodiments, the present invention relates to any one of the aforementioned methods, wherein at least about 5% of R¹ is

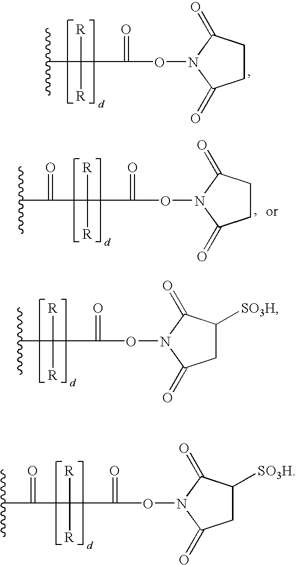

In certain embodiments, the present invention relates to any one of the aforementioned methods, wherein at least about 10% of R¹ is

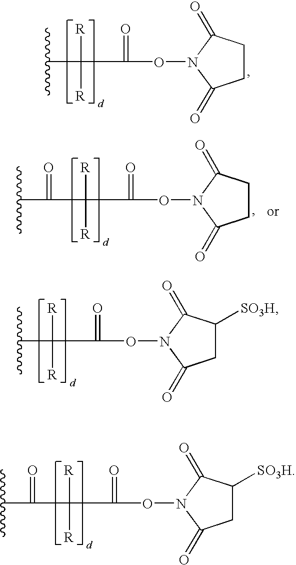

In certain embodiments, the present invention relates to any one of the aforementioned methods, wherein at least about 25% of R¹ is

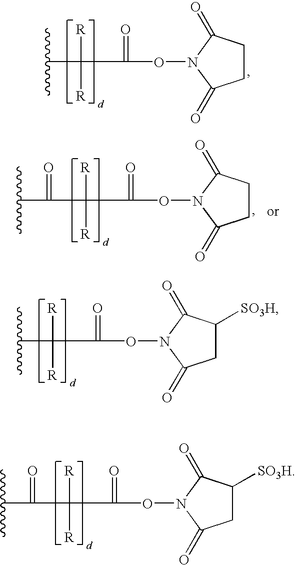

In certain embodiments, the present invention relates to any one of the aforementioned methods, wherein at least about 50% of R¹ is

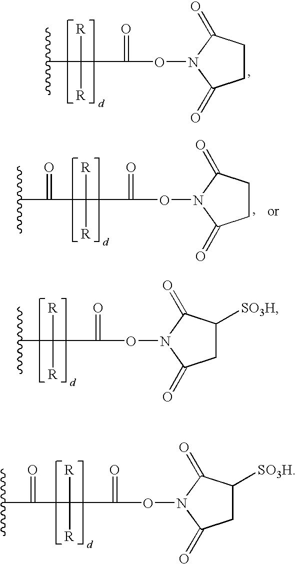

In certain embodiments, the present invention relates to any one of the aforementioned methods, wherein R² is an electron pair.

In certain embodiments, the present invention relates to any one of the aforementioned methods, wherein R⁶ is hydrogen.

In certain embodiments, the present invention relates to any one of the aforementioned methods, wherein R⁷ is hydrogen.

In certain embodiments, the present invention relates to any one of the aforementioned methods, wherein R⁸ is hydrogen.

In certain embodiments, the present invention relates to any one of the aforementioned methods, wherein R⁹ is hydrogen.

In certain embodiments, the present invention relates to any one of the aforementioned methods, wherein d is 1-8 inclusive.

In certain embodiments, the present invention relates to any one of the aforementioned methods, wherein n is 1 or 2.

In certain embodiments, the present invention relates to any one of the aforementioned methods, wherein the sum or y and z is an integer from about 50 to about 200.

In certain embodiments, the present invention relates to any one of the aforementioned methods, wherein y is an integer in the range of about 2 to about 100.

In certain embodiments, the present invention relates to any one of the aforementioned methods, wherein the second composition comprises a compound of formula III selected from the group consisting

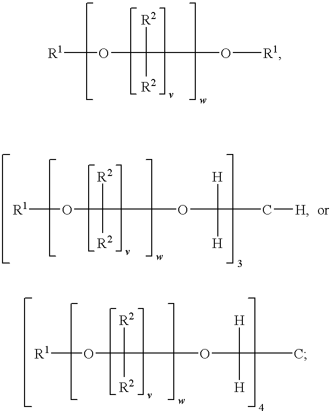

wherein, independently for each occurrence,

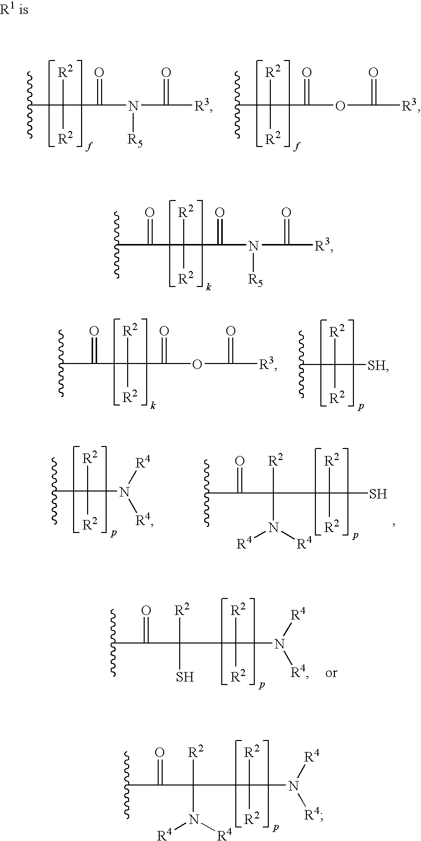

R² is hydrogen, alkyl, or halogen;

R³ is

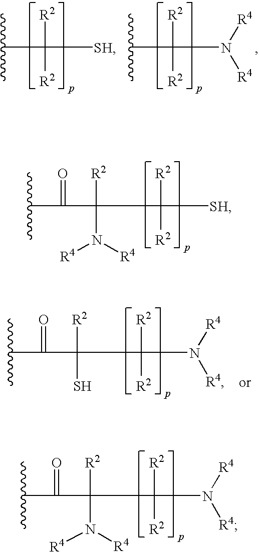

R⁴ is hydrogen, alkyl, aryl, aralkyl,

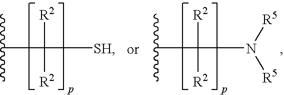

R⁵ is hydrogen, or alkyl;

f is an integer from 1 to 25 inclusive;

k is an integer from 1 to 25 inclusive;

p is an integer from 1 to 5 inclusive;

v is an integer from 2 to 4 inclusive; and

w is an integer from 5 to 1,000 inclusive.

In certain embodiments, the present invention relates to any one of the aforementioned methods, wherein the compound of formula III is

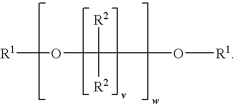

In certain embodiments, the present invention relates to any one of the aforementioned methods, wherein R² is hydrogen.

In certain embodiments, the present invention relates to any one of the aforementioned methods, wherein R⁴ is hydrogen.

In certain embodiments, the present invention relates to any one of the aforementioned methods, wherein R⁵ is hydrogen.

In certain embodiments, the present invention relates to any one of the aforementioned methods, wherein f is an integer from 1 to 9 inclusive.

In certain embodiments, the present invention relates to any one of the aforementioned methods, wherein k is an integer from 1 to 9 inclusive.

In certain embodiments, the present invention relates to any one of the aforementioned methods, wherein the first amount of time is about 2 minutes.

In certain embodiments, the present invention relates to any one of the aforementioned methods, wherein the first amount of time is about 1 minute.

In certain embodiments, the present invention relates to any one of the aforementioned methods, wherein the first amount of time is about 30 seconds.

In certain embodiments, the present invention relates to any one of the aforementioned methods, wherein the first amount of time is about 10 seconds.

### Use of a Polymerization Agent, and Visible or Ultraviolet Light, to Form a Hydrogel

In certain embodiments, the present invention relates to any one of the aforementioned methods, further comprising the steps of:

exposing the first material, the second material, or both the first material and the second material, to a third composition, thereby forming a photo-polymerizable material; and exposing the photo-polymerizable material to ultraviolet or visible light, thereby polymerizing the photo-polymerizable material;

wherein said third composition comprises a compound of formula V:

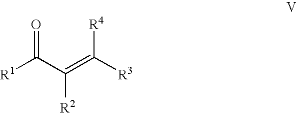

wherein, independently for each occurrence,

R¹ is a halogen,

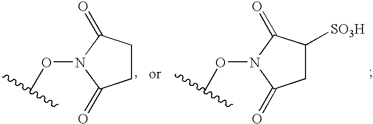

R² hydrogen, alkyl, aryl, or aralkyl;

R³ hydrogen, alkyl, aryl, or aralkyl; and

R⁴ is hydrogen, alkyl, aryl, or aralkyl.

In certain embodiments, the present invention relates to any one of the aforementioned methods, wherein said compound of formula V is

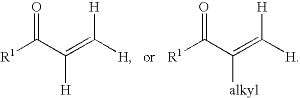

In certain embodiments, the present invention relates to any one of the aforementioned methods, wherein said compound of formula V is

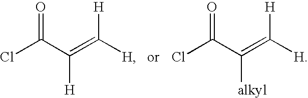

In certain embodiments, the present invention relates to any one of the aforementioned methods, wherein said photo-polymerizable material is treated with ultraviolet light only.

In certain embodiments, the present invention relates to any one of the aforementioned methods, wherein said photo-polymerizable material is treated with visible light only; and said method further comprises the step of exposing said photo-polymerizable material to a photoinitiator.

In certain embodiments, the present invention relates to any one of the aforementioned methods, wherein said photoinitiator is eosin y.

In certain embodiments, the present invention relates to any one of the aforementioned methods, wherein the step of covering the defect, wound, or void with a first material comprises the steps of:

applying a composition to the defect, wound or void; and

treating the composition with ultraviolet light or visible light sufficient to polymerize said the composition, thereby forming the first material.

In certain embodiments, the present invention relates to any one of the aforementioned methods, wherein the step of covering the first material with a second material comprises the steps of:

applying a composition to cover the first material; and

treating the composition with ultraviolet light or visible light sufficient to polymerize said the composition, thereby forming the second material.

In certain embodiments, the present invention relates to any one of the aforementioned methods, wherein said composition comprises a compound of formula I:

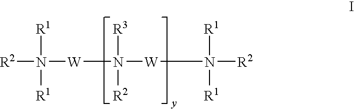

wherein, independently for each occurrence,

W is —CR₂—, —CR₂CR₂—, —CR₂CR₂CR₂—, —CR₂CR₂CR₂CR₂—, or —CR₂CR₂CR₂CR₂CR₂—;

R is hydrogen, methyl, ethyl, n-propyl, or i-propyl;

R¹ is hydrogen, alkyl, alkenyl, alkynyl,

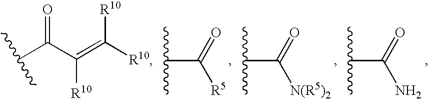

or a carbohydrate radical;

Y is —CR₂—, —CR₂CR₂—, —CR₂CR₂CR₂—, —CR₂CR₂CR₂CR₂—, or —CR₂CR₂CR₂CR₂CR₂—;

R² is an electron pair, hydrogen, alkyl, or aralkyl, provided that a pharmaceutically acceptable counter ion is present when R² is not an electron pair;

R³ is hydrogen, or

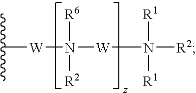

R⁴ is hydrogen, alkyl, alkoxyl, halogen, or aralkyl;

R⁵ is hydrogen, alkyl, aryl, or aralkyl;

R⁶ is hydrogen, or

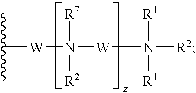

R⁷ is hydrogen, or

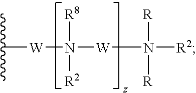

R⁸ is hydrogen, or

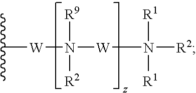

R⁹ is hydrogen, or

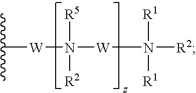

R¹⁰ is hydrogen, alkyl, aryl, or aralkyl;

d is an integer from 1 to 10 inclusive;

n is an integer from 1 to 4 inclusive;

y is an integer from 5 to 40,000 inclusive; and

z is an integer from 0 to 20,000 inclusive;

provided that at least about 5% of R¹ is

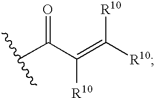

and the sum of y and z is less than about 50,000; and

In certain embodiments, the present invention relates to any one of the aforementioned methods, wherein said composition comprises a compound of formula III selected from the group consisting of

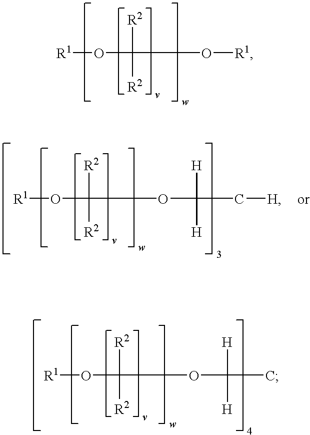

wherein, independently for each occurrence,

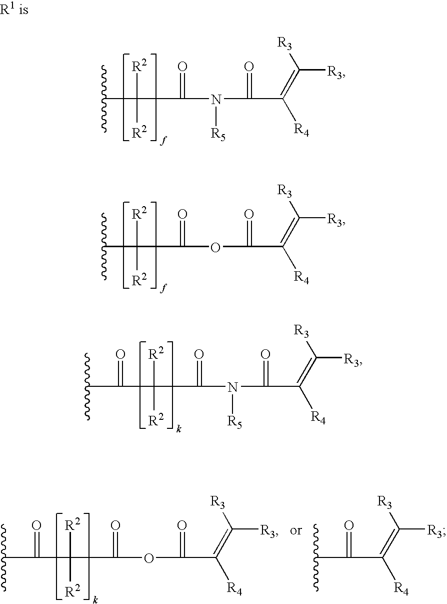

R² is hydrogen, alkyl, or halogen;

R³ is hydrogen, alkyl, aryl, or aralkyl;

R⁴ is hydrogen, alkyl, aryl, or aralkyl;

R⁵ is hydrogen, alkyl, aryl, or aralkyl;

f is an integer from 1 to 25 inclusive;

k is an integer from 1 to 25 inclusive;

v is an integer from 2 to 4 inclusive; and

w is an integer from 5 to 1,000 inclusive.

In certain embodiments, the present invention relates to any one of the aforementioned methods, wherein R¹ is

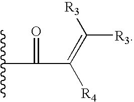

In certain embodiments, the present invention relates to any one of the aforementioned methods, wherein R¹ is

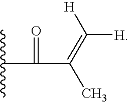

In certain embodiments, the present invention relates to any one of the aforementioned methods, wherein said composition is treated with ultraviolet light only.

In certain embodiments, the present invention relates to any one of the aforementioned methods, wherein said composition is treated with visible light only; and said method further comprises the step of exposing said composition to a photoinitiator.

In certain embodiments, the present invention relates to any one of the aforementioned methods, wherein said photoinitiator is eosin y.

### Examples of Types of Patients and Wounds

In certain embodiments, the present invention relates to the aforementioned method, wherein said patient is a primate, bovine, equine, feline, or canine.

In certain embodiments, the present invention relates to the aforementioned method, wherein said patient is a human.

In certain embodiments, the present invention relates to the aforementioned method, wherein said wound is a located in the dura.

In certain embodiments, the present invention relates to the aforementioned method, wherein said wound is a located in the lung tissue.

In certain embodiments, the present invention relates to the aforementioned method, wherein said wound is a tissue plane.

In certain embodiments, the present invention relates to the aforementioned method, wherein said wound is in a vein or artery.

In certain embodiments, the present invention relates to any one of the aforementioned methods, wherein said wound is an ophthalmic wound.

In certain embodiments, the present invention relates to any one of the aforementioned methods, wherein said wound is an epithelial defect, corneal incision, corneal laceration, corneal perforation, corneal ulceration, retinal hole, filtering bleb, corneal transplant, trabeculectomy incision, sclerotomy incision, blepharoplasty, or skin incision.

In certain embodiments, the present invention relates to any one of the aforementioned methods, wherein said wound is in the liver.

In certain embodiments, the present invention relates to any one of the aforementioned methods, wherein said wound is in the lung.

In certain embodiments, the present invention relates to any one of the aforementioned methods, wherein said wound is in the heart.

In certain embodiments, the present invention relates to any one of the aforementioned methods, wherein said wound is the pancreas.

In certain embodiments, the present invention relates to any one of the aforementioned methods, wherein said wound is in the dura matter.

In certain embodiments, the present invention relates to any one of the aforementioned methods, wherein said wound is in an artery or vein.

In certain embodiments, the present invention relates to any one of the aforementioned methods, wherein said wound is associated with a mastectomy.

In certain embodiments, the present invention relates to any one of the aforementioned methods, wherein said wound is associated with a lumpectomy.

In certain embodiments, the present invention relates to any one of the aforementioned methods, wherein said wound is associated with abdominoplasty.

In certain embodiments, the present invention relates to any one of the aforementioned methods, wherein said wound is associated with rhytidectomy or rhinoplasty.

In certain embodiments, the present invention relates to any one of the aforementioned methods, wherein said wound is associated with mammaplasty.

In certain embodiments, the present invention relates to any one of the aforementioned methods, wherein said wound is associated with a forehead or buttocks lift.

In certain embodiments, the present invention relates to any one of the aforementioned methods, wherein said wound is associated with a skin graft.

In certain embodiments, the present invention relates to any one of the aforementioned methods, wherein said wound is associated with a biopsy closure.

In certain embodiments, the present invention relates to any one of the aforementioned methods, wherein said wound is associated with a cleft-palate reconstruction.

In certain embodiments, the present invention relates to any one of the aforementioned methods, wherein said wound is associated with hernia or groin repair.

In certain embodiments, the present invention relates to any one of the aforementioned methods, wherein said wound is associated with a Caesarean section.

In certain embodiments, the present invention relates to any one of the aforementioned methods, wherein said wound is associated with a laparoscopic trocar repair.

In certain embodiments, the present invention relates to any one of the aforementioned methods, wherein said wound is associated with a vaginal tear repair.

In certain embodiments, the present invention relates to any one of the aforementioned methods, wherein said wound is associated with gastrointestinal anastomosis.

In certain embodiments, the present invention relates to any one of the aforementioned methods, wherein said wound is associated with prostatectomy urethral-bladder anastomosis.

In certain embodiments, the present invention relates to any one of the aforementioned methods, wherein said wound is associated with a myocardial infarction.

In certain embodiments, the present invention relates to any one of the aforementioned methods, wherein said wound is associated with a perforated eardrum.

In certain embodiments, the present invention relates to any one of the aforementioned methods, wherein said wound is associated with a partially penetrating keratoplasty procedure.

In certain embodiments, the present invention relates to any one of the aforementioned methods, wherein said wound is in the dura mater of the nervous system.

In certain embodiments, the present invention relates to any one of the aforementioned methods, wherein said wound is in a cardiac artery or cardiac vein.

In certain embodiments, the present invention relates to any one of the aforementioned methods, wherein said wound is in a parenchymal organ.

In certain embodiments, the present invention relates to any one of the aforementioned methods, wherein said wound is in the spleen.

In certain embodiments, the present invention relates to any one of the aforementioned methods, wherein said wound is in the gastrointestinal system.

In certain embodiments, the present invention relates to any one of the aforementioned methods, wherein said wound is in the genitourinary system.

In certain embodiments, the present invention relates to any one of the aforementioned methods, wherein said wound is associated with mentoplasty.

In certain embodiments, the present invention relates to any one of the aforementioned methods, wherein said wound is associated with brachioplasty.

In certain embodiments, the present invention relates to any one of the aforementioned methods, wherein said wound is associated with gynecomastia reduction.

Use of a Mesh or Scaffolding in the Masking and/or Covering Material

As mentioned above, masking and covering materials of the present invention could be used in combination with a degradable or nondegradable mesh to secure a tissue site. The combination of the mesh and the adhesive provides for improved strength. This protocol is particular useful when the area of tissue repair is large.

In certain embodiments a polymer mesh impregnated with a suitable anti-adhesion masking material (i.e., one which will dissolve in a short period of time after the application of the covering material) may be used. In such an embodiment, the masking material would be cut to size and then applied to the wound, void, or damaged tissue and an area which extends around the edge of the wound, void, or damaged tissue. The covering material would then be applied over the solid polymer material and onto the uncovered area around the wound, void, or damaged tissue site to form a homogeneous hydrogel film over the entire site. Over several hours, the under lying anti-adhesive film would dissolve leaving only the covering material and the polymer mesh as the protective barrier over the wound, void, or damaged tissue site. At this point the covering material would be connected to the periphery as an adhesive, but not be adhered to the wound, void, or damaged tissue site itself.

In yet other embodiments, a polymer mesh which may be applied over a suitable anti-adhesion masking material (i.e., a masking material which will dissolve in a short period of time after the application of the covering material). In such embodiments, the material would be cut to size and then applied to the wound, void, or damaged tissue site and an area which extends around the edge of the wound, void, or damaged tissue site. The covering would then be applied over the solid polymer material and onto the uncovered area around the wound, void, or damaged tissue site to form a homogeneous covering film over the entire site. Over several hours, the under lying anti-adhesive masking material would dissolve leaving only the cover material and the polymer mesh as the protective barrier over the wound, void, or damaged tissue site. In this case, the polymer mesh would be incorporated into the in situ polymerized covering material and would become an integral part of the material. Alternatively, the polymer mesh may be biodegradable, leaving only the covering material over the wound, void, or damaged tissue. In either case, at this point the covering material would be connected to the periphery as an adhesive, but not be adhered to the wound, void, or damaged tissue site itself.

In certain instances, the scaffold is placed in the wound, void, or damaged tissue site and the masking material is then applied to the wound, void, or damaged tissue; a covering composition is then subsequently applied. This approach provides that the tissue and the scaffold are secure in the wound, void, or damaged tissue site. Alternatively, the masking material is added to a wound, void, or damaged tissue and a covering composition comprising a scaffold is placed over the masking material.

In certain embodiments, the mesh may be formed from methylmethacrylate, mersilene, silicone, Teflon®, Dacron®, polyethylene, polyester, titanium-Dacron®, hydroxylapatite, or combinations thereof. In particular, polypropylene mesh has been used with good results in general surgery, as well as in plastic reconstructive, urological, gynecological, and thoracic surgeries. In certain embodiments, the mesh comprises polypropylene or polyester.

As mentioned above, one aspect of the invention relates to a masking or covering composition comprising a degradable scaffold. In certain instances, the degradable scaffold comprises a biodegradable polymer. In certain instances, the degradable scaffold comprises poly(glycolic acid), poly(lactic acid), or copolymers thereof. In certain instances, the degradable scaffold comprises poly(lactic acid). In certain instances, the biodegradable polymer has a weight average molecular weight of about 500 g/mol to about 500,000 g/mol. In certain instances, the biodegradable polymer has a weight average molecular weight of about 500 g/mol to about 100,000 g/mol.

In still other embodiments, the polymer meshes as described above, in which the surface of the polymer mesh has been modified such that it contains nucleophilic sites, namely amine functional groups, which can react with the electrophilic portion of the in situ polymerizing polymer material, can be used. In effect, covalently bonding the mesh to the polymerizing material.

Use of Pre-Formed Masking and/or Covering Materials

As mentioned above, another aspect of the invention relates to a method of repairing a wound, void, or tissue in a patient, comprising the steps of applying a preformed hydrogel of the invention to the wound, void, or tissue of a patient. Either the masking composition, the covering composition, or both, may be pre-formed hydrogels. In certain instances, the preformed hydrogel is placed in the tissue site and then the covering composition is added. Alternatively, a masking material (e.g., a hydrogel) is applied to the wound, void, or damaged tissue site, the preformed hydrogel is added as the covering composition, and then the wound, void, or damaged tissue site is thereby closed.

In certain embodiments, the preformed masking material can either be the same material as the covering material used for the secondary bandaging process or it may be a gel which degrades faster than the covering material. For example, a preformed gel can be cut to size and placed over a wound. Since there will be no overspray or “over painting”, the doctor can be less precise about applying this preformed masking material. In certain embodiments, the same material is used for both parts of the wound coverage (the masking material and the covering material).

In certain embodiments, a polymer film which will dissolve in a short period of time after the application of the covering material (e.g., a hydrogel), is used as the masking material. It is well known that certain materials can be used as air solid barrier, and these materials, upon exposure to water the material, dissolve. In this case, the masking material would be cut to size and then applied to the wound site and an area which extends around the edge of the wound site. The covering material would then be applied over the solid polymer material and onto the uncovered area around the wound site to form a homogeneous covering film over the entire site. Over several hours, the under lying polymer film would dissolve leaving only the covering material as the protective barrier over the wound site. At this point the covering material would be connected to the periphery as an adhesive, but not be adhered to the wound site itself.

### Pharmaceutically Acceptable Salts

As set out herein, certain embodiments of the present masking materials and covering materials may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. See, for example, J. Pharm. Sci. 1977, 66, 1-19.

The pharmaceutically acceptable salts of the subject compounds include the conventional nontoxic salts or ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

### Biologically Active Agents and Pharmaceutically Active Agents

In certain instances, biologically active agents may be incorporated in the masking material, the covering material, or both. Active agents amenable for use in the compositions of the present invention include growth factors, such as transforming growth factors (TGFs), fibroblast growth factors (FGFs), platelet derived growth factors (PDGFs), epidermal growth factors (EGFs), connective tissue ctivated peptides (CTAPs), osteogenic factors, and biologically active analogs, fragments, and derivatives of such growth factors. Members of the transforming growth factor (TGF) supergene family, which are multifunctional regulatory proteins, are particularly preferred. Members of the TGF supergene family include the beta transforming growth factors (for example, TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

In addition to the biological active agents discussed above, a large number of pharmaceutical agents are known in the art and are amenable for use in the compositions of the invention. The term “pharmaceutical agent” includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Non-limiting examples of broad categories of useful pharmaceutical agents include the following therapeutic categories: anabolic agents, antacids, anti-asthmatic agents, anti-cholesterolemic and anti-lipid agents, anti-coagulants, anti-convulsants, anti-diarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives, hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, and prodrugs.

More specifically, non-limiting examples of useful pharmaceutical agents include the following therapeutic categories: analgesics, such as nonsteroidal anti-inflammatory drugs, opiate agonists and salicylates; antihistamines, such as H1-blockers and H2-blockers; anti-infective agents, such as anthelmintics, antianaerobics, antibiotics, aminoglycoside antibiotics, antifungal antibiotics, cephalosporin antibiotics, macrolide antibiotics, miscellaneous beta-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, and urinary anti-infectives; antineoplastic agents, such as alkylating agents, nitrogen mustard alkylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics; autonomic agents, such as anticholinergics, antimuscarinic anticholinergics, ergot alkaloids, parasympathomimetics, cholinergic agonist parasympathomimetics, cholinesterase inhibitor para-sympathomimetics, sympatholytics, alpha-blocker sympatholytics, beta-blocker sympatholytics, sympathomimetics, and adrenergic agonist sympathomimetics; cardiovascular agents, such as antianginals, beta-blocker antianginals, calcium-channel blocker antianginals, nitrate antianginals, antiarrhythmics, cardiac glycoside antiarrhythmics, class I antiarrhythmics, class II antiarrhythmics, class III antiarrhythmics, class IV antiarrhythmics, antihypertensive agents, alpha-blocker antihypertensives, angiotensin-converting enzyme inhibitor (ACE inhibitor) antihypertensives, beta-blocker antihypertensives, calcium-channel blocker antihypertensives, central-acting adrenergic antihypertensives, diuretic antihypertensive agents, peripheral vasodilator antihypertensives, antilipemics, bile acid sequestrant antilipemics, HMG-CoA reductase inhibitor antilipemics, inotropes, cardiac glycoside inotropes, and thrombolytic agents; dermatological agents, such as antihistamines, anti-inflammatory agents, corticosteroid anti-inflammatory agents, antipruritics/local anesthetics, topical anti-infectives, antifungal topical anti-infectives, antiviral topical anti-infectives, and topical antineoplastics; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H₂-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hematological agents, such as antianemia agents, hematopoietic antianemia agents, coagulation agents, anticoagulants, hemostatic coagulation agents, platelet inhibitor coagulation agents, thrombolytic enzyme coagulation agents, and plasma volume expanders; hormones and hormone modifiers, such as abortifacients, adrenal agents, corticosteroid adrenal agents, androgens, anti-androgens, antidiabetic agents, sulfonylurea antidiabetic agents, antihypoglycemic agents, oral contraceptives, progestin contraceptives, estrogens, fertility agents, oxytocics, parathyroid agents, pituitary hormones, progestins, antithyroid agents, thyroid hormones, and tocolytics; immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immuno-suppressive anti-inflammatory agents, nonsteroidal anti-inflammatory drugs (NSAIDs), salicylate anti-inflammatory agents, skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists; ophthalmic agents, such as anti-glaucoma agents, beta-blocker anti-gluacoma agents, miotic anti-glaucoma agents, mydriatics, adrenergic agonist mydriatics, antimuscarinic mydriatics, ophthalmic anesthetics, ophthalmic anti-infectives, ophthalmic aminoglycoside anti-infectives, ophthalmic macrolide anti-infectives, ophthalmic quinolone anti-infectives, ophthalmic sulfonamide anti-infectives, ophthalmic tetracycline anti-infectives, ophthalmic anti-inflammatory agents, ophthalmic corticosteroid anti-inflammatory agents, and ophthalmic nonsteroidal anti-inflammatory drugs (NSAIDs); psychotropic agents, such as antidepressants, heterocyclic antidepressants, monoamine oxidase inhibitors (MAOIs), selective serotonin re-uptake inhibitors (SSRIs), tricyclic antidepressants, antimanics, antipsychotics, phenothiazine antipsychotics, anxiolytics, sedatives, and hypnotics, barbiturate sedatives and hypnotics, benzodiazepine anxiolytics, sedatives, and hypnotics, and psychostimulants; respiratory agents, such as antitussives, bronchodilators, adrenergic agonist bronchodilators, antimuscarinic bronchodilators, expectorants, mucolytic agents, respiratory anti-inflammatory agents, and respiratory corticosteroid anti-inflammatory agents; toxicology agents, such as antidotes, heavy metal antagonists/chelating agents, substance abuse agents, deterrent substance abuse agents, and withdrawal substance abuse agents; minerals; and vitamins, such as vitamin A, vitamin B, vitamin C, vitamin D, vitamin E, and vitamin K.

Preferred classes of useful pharmaceutical agents from the above categories include: (1) nonsteroidal anti-inflammatory drugs (NSAIDs) analgesics, such as diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H₁-blocker antihistamines, such as clemastine and terfenadine; (5) H₂-blocker antihistamines, such as cimetidine, famotidine, nizadine, and ranitidine; (6) anti-infective agents, such as mupirocin; (7) antianaerobic anti-infectives, such as chloramphenicol and clindamycin; (8) antifungal antibiotic anti-infectives, such as amphotericin b, clotrimazole, fluconazole, and ketoconazole; (9) macrolide antibiotic anti-infectives, such as azithromycin and erythromycin; (10) miscellaneous beta-lactam antibiotic anti-infectives, such as aztreonam and imipenem; (11) penicillin antibiotic anti-infectives, such as nafcillin, oxacillin, penicillin G, and penicillin V; (12) quinolone antibiotic anti-infectives, such as ciprofloxacin and norfloxacin; (13) tetracycline antibiotic anti-infectives, such as doxycycline, minocycline, and tetracycline; (14) antituberculosis antimycobacterial anti-infectives such as isoniazid (INH), and rifampin; (15) antiprotozoal anti-infectives, such as atovaquone and dapsone; (16) antimalarial antiprotozoal anti-infectives, such as chloroquine and pyrimethamine; (17) anti-retroviral anti-infectives, such as ritonavir and zidovudine; (18) antiviral anti-infective agents, such as acyclovir, ganciclovir, interferon alfa, and rimantadine; (19) alkylating antineoplastic agents, such as carboplatin and cisplatin; (20) nitrosourea alkylating antineoplastic agents, such as carmustine (BCNU); (21) antimetabolite antineoplastic agents, such as methotrexate; (22) pyrimidine analog antimetabolite antineoplastic agents, such as fluorouracil (5-FU) and gemcitabine; (23) hormonal antineoplastics, such as goserelin, leuprolide, and tamoxifen; (24) natural antineoplastics, such as aldesleukin, interleukin-2, docetaxel, etoposide (VP-16), interferon alfa, paclitaxel, and tretinoin (ATRA); (25) antibiotic natural antineoplastics, such as bleomycin, dactinomycin, daunorubicin, doxorubicin, and mitomycin; (26) vinca alkaloid natural antineoplastics, such as vinblastine and vincristine; (27) autonomic agents, such as nicotine; (28) anticholinergic autonomic agents, such as benztropine and trihexyphenidyl; (29) antimuscarinic anticholinergic autonomic agents, such as atropine and oxybutynin; (30) ergot alkaloid autonomic agents, such as bromocriptine; (31) cholinergic agonist parasympathomimetics, such as pilocarpine; (32) cholinesterase inhibitor parasympathomimetics, such as pyridostigmine; (33) alpha-blocker sympatholytics, such as prazosin; (34) beta-blocker sympatholytics, such as atenolol; (35) adrenergic agonist sympathomimetics, such as albuterol and dobutamine; (36) cardiovascular agents, such as aspirin (ASA) (enteric coated ASA); (37) beta-blocker antianginals, such as atenolol and propranolol; (38) calcium-channel blocker antianginals, such as nifedipine and verapamil; (39) nitrate antianginals, such as isosorbide dinitrate (ISDN); (40) cardiac glycoside antiarrhythmics, such as digoxin; (41) class I anti-arrhythmics, such as lidocaine, mexiletine, phenyloin, procainamide, and quinidine; (42) class II antiarrhythmics, such as atenolol, metoprolol, propranolol, and timolol; (43) class III antiarrhythmics, such as amiodarone; (44) class IV antiarrhythmics, such as diltiazem and verapamil; (45) α-blocker antihypertensives, such as prazosin; (46) angiotensin-converting enzyme inhibitor (ACE inhibitor) antihypertensives, such as captopril and enalapril; (47) β-blocker antihypertensives, such as atenolol, metoprolol, nadolol, and propanolol; (48) calcium-channel blocker antihypertensive agents, such as diltiazem and nifedipine; (49) central-acting adrenergic antihypertensives, such as clonidine and methyldopa; (50) diurectic antihypertensive agents, such as amiloride, furosemide, hydrochlorothiazide (HCTZ), and spironolactone; (51) peripheral vasodilator antihypertensives, such as hydralazine and minoxidil; (52) antilipemics, such as gemfibrozil and probucol; (53) bile acid sequestrant antilipemics, such as cholestyramine; (54) HMG-CoA reductase inhibitor antilipemics, such as lovastatin and pravastatin; (55) inotropes, such as aminone, dobutamine, and dopamine; (56) cardiac glycoside inotropes, such as digoxin; (57) thrombolytic agents, such as alteplase (TPA), anistreplase, streptokinase, and urokinase; (58) dermatological agents, such as colchicine, isotretinoin, methotrexate, minoxidil, tretinoin (ATRA); (59) dermatological corticosteroid anti-inflammatory agents, such as betamethasone and dexamethasone; (60) antifungal topical anti-infectives, such as amphotericin B, clotrimazole, miconazole, and nystatin; (61) antiviral topical anti-infectives, such as acyclovir; (62) topical antineoplastics, such as fluorouracil (5-FU); (63) electrolytic and renal agents, such as lactulose; (64) loop diuretics, such as furosemide; (65) potassium-sparing diuretics, such as triamterene; (66) thiazide diuretics, such as hydro-chlorothiazide (HCTZ); (67) uricosuric agents, such as probenecid; (68) enzymes such as RNase and DNase; (69) thrombolytic enzymes, such as alteplase, anistreplase, streptokinase and urokinase; (70) antiemetics, such as prochlorperazine; (71) salicylate gastrointestinal anti-inflammatory agents, such as sulfasalazine; (72) gastric acid-pump inhibitor anti-ulcer agents, such as omeprazole; (73) H₂-blocker anti-ulcer agents, such as cimetidine, famotidine, nizatidine, and ranitidine; (74) digestants, such as pancrelipase; (75) prokinetic agents, such as erythromycin; (76) opiate agonist intravenous anesthetics such as fentanyl; (77) hematopoietic antianemia agents, such as erythropoietin, filgrastim (G-CSF), and sargramostim (GM-CSF); (78) coagulation agents, such as antihemophilic factors 1-10 (AHF 1-10); (79) anticoagulants, such as warfarin; (80) thrombolytic enzyme coagulation agents, such as alteplase, anistreplase, streptokinase and urokinase; (81) hormones and hormone modifiers, such as bromocriptine; (82) abortifacients, such as methotrexate; (83) antidiabetic agents, such as insulin; (84) oral contraceptives, such as estrogen and progestin; (85) progestin contraceptives, such as levonorgestrel and norgestrel; (86) estrogens such as conjugated estrogens, diethylstilbestrol (DES), estrogen (estradiol, estrone, and estropipate); (87) fertility agents, such as clomiphene, human chorionic gonadatropin (HCG), and menotropins; (88) parathyroid agents such as calcitonin; (89) pituitary hormones, such as desmopressin, goserelin, oxytocin, and vasopressin (ADH); (90) progestins, such as medroxyprogesterone, norethindrone, and progesterone; (91) thyroid hormones, such as levothyroxine; (92) immunobiologic agents, such as interferon beta-1b and interferon gamma-1b; (93) immunoglobulins, such as immune globulin IM, IMIG, IGIM and immune globulin IV, IVIG, IGIV; (94) amide local anesthetics, such as lidocaine; (95) ester local anesthetics, such as benzocaine and procaine; (96) musculoskeletal corticosteroid anti-inflammatory agents, such as beclomethasone, betamethasone, cortisone, dexamethasone, hydrocortisone, and prednisone; (97) musculoskeletal anti-inflammatory immunosuppressives, such as azathioprine, cyclophosphamide, and methotrexate; (98) musculoskeletal nonsteroidal anti-inflammatory drugs (NSAIDs), such as diclofenac, ibuprofen, ketoprofen, ketorlac, and naproxen; (99) skeletal muscle relaxants, such as baclofen, cyclobenzaprine, and diazepam; (100) reverse neuromuscular blocker skeletal muscle relaxants, such as pyridostigmine; (101) neurological agents, such as nimodipine, riluzole, tacrine and ticlopidine; (102) anticonvulsants, such as carbamazepine, gabapentin, lamotrigine, phenyloin, and valproic acid; (103) barbiturate anticonvulsants, such as phenobarbital and primidone; (104) benzodiazepine anticonvulsants, such as clonazepam, diazepam, and lorazepam; (105) anti-parkisonian agents, such as bromocriptine, levodopa, carbidopa, and pergolide; (106) anti-vertigo agents, such as meclizine; (107) opiate agonists, such as codeine, fentanyl, hydromorphone, methadone, and morphine; (108) opiate antagonists, such as naloxone; (109) β-blocker anti-glaucoma agents, such as timolol; (110) miotic anti-glaucoma agents, such as pilocarpine; (111) ophthalmic aminoglycoside antiinfectives, such as gentamicin, neomycin, and tobramycin; (112) ophthalmic quinolone anti-infectives, such as ciprofloxacin, norfloxacin, and ofloxacin; (113) ophthalmic corticosteroid anti-inflammatory agents, such as dexamethasone and prednisolone; (114) ophthalmic nonsteroidal anti-inflammatory drugs (NSAIDs), such as diclofenac; (115) antipsychotics, such as clozapine, haloperidol, and risperidone; (116) benzodiazepine anxiolytics, sedatives and hypnotics, such as clonazepam, diazepam, lorazepam, oxazepam, and prazepam; (117) psychostimulants, such as methylphenidate and pemoline; (118) antitussives, such as codeine; (119) bronchodilators, such as theophylline; (120) adrenergic agonist bronchodilators, such as albuterol; (121) respiratory corticosteroid anti-inflammatory agents, such as dexamethasone; (122) antidotes, such as flumazenil and naloxone; (123) heavy metal antagonists/chelating agents, such as penicillamine; (124) deterrent substance abuse agents, such as disulfuram, naltrexone, and nicotine; (125) withdrawal substance abuse agents, such as bromocriptine; (126) minerals, such as iron, calcium, and magnesium; (127) vitamin B compounds, such as cyanocobalamin (vitamin B12) and niacin (vitamin B3); (128) vitamin C compounds, such as ascorbic acid; and (129) vitamin D compounds, such as calcitriol.

In addition to the foregoing, the following less common drugs may also be used: chlorhexidine; estradiol cypionate in oil; estradiol valerate in oil; flurbiprofen; flurbiprofen sodium; ivermectin; levodopa; nafarelin; and somatropin. Further, the following drugs may also be used: recombinant beta-glucan; bovine immunoglobulin concentrate; bovine superoxide dismutase; the formulation comprising fluorouracil, epinephrine, and bovine collagen; recombinant hirudin (r-Hir), HIV-1 immunogen; human anti-TAC antibody; recombinant human growth hormone (r-hGH); recombinant human hemoglobin (r-Hb); recombinant human mecasermin (r-IGF-1); recombinant interferon β-1a; lenograstim (G-CSF); olanzapine; recombinant thyroid stimulating hormone (r-TSH); and topotecan.

Further still, the following intravenous products may be used: acyclovir sodium; aldesleukin; atenolol; bleomycin sulfate, human calcitonin; salmon calcitonin; carboplatin; carmustine; dactinomycin, daunorubicin HCl; docetaxel; doxorubicin HCl; epoetin alfa; etoposide (VP-16); fluorouracil (5-FU); ganciclovir sodium; gentamicin sulfate; interferon alfa; leuprolide acetate; meperidine HCl; methadone HCl; methotrexate sodium; paclitaxel; ranitidine HCl; vinblastin sulfate; and zidovudine (AZT).

Further specific examples of useful pharmaceutical agents from the above categories include: (a) anti-neoplastics such as androgen inhibitors, antimetabolites, cytotoxic agents, and immunomodulators; (b) anti-tussives such as dextromethorphan, dextromethorphan hydrobromide, noscapine, carbetapentane citrate, and chlorphedianol hydrochloride; (c) antihistamines such as chlorpheniramine maleate, phenindamine tartrate, pyrilamine maleate, doxylamine succinate, and phenyltoloxamine citrate; (d) decongestants such as phenylephrine hydrochloride, phenylpropanolamine hydrochloride, pseudoephedrine hydrochloride, and ephedrine; (e) various alkaloids such as codeine phosphate, codeine sulfate and morphine; (f) mineral supplements such as potassium chloride, zinc chloride, calcium carbonates, magnesium oxide, and other alkali metal and alkaline earth metal salts; (g) ion exchange resins such as cholestryramine; (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; (k) expectorants such as guaifenesin; (l) antacids such as aluminum hydroxide and magnesium hydroxide; (m) biologicals such as peptides, polypeptides, proteins and amino acids, hormones, interferons or cytokines, and other bioactive peptidic compounds, such as interleukins 1-18 including mutants and analogues, RNase, DNase, luteinizing hormone releasing hormone (LHRH) and analogues, gonadotropin releasing hormone (GnRH), transforming growth factor-.beta. (TGF-beta), fibroblast growth factor (FGF), tumor necrosis factor-alpha & beta (TNF-alpha & beta), nerve growth factor (NGF), growth hormone releasing factor (GHRF), epidermal growth factor (EGF), fibroblast growth factor homologous factor (FGFHF), hepatocyte growth factor (HGF), insulin growth factor (IGF), invasion inhibiting factor-2 (IIF-2), bone morphogenetic proteins 1-7 (BMP 1-7), somatostatin, thymosin-alpha-1, gamma-globulin, superoxide dismutase (SOD), complement factors, hGH, tPA, calcitonin, ANF, EPO and insulin; and (n) anti-infective agents such as antifungals, anti-virals, antiseptics and antibiotics.

Alternatively, the pharmaceutical agent may be a radiosensitizer, such as metoclopramide, sensamide or neusensamide (manufactured by Oxigene); profiromycin (made by Vion); RSR13 (made by Allos); Thymitaq (made by Agouron), etanidazole or lobenguane (manufactured by Nycomed); gadolinium texaphrin (made by Pharmacyclics); BuDR/Broxine (made by NeoPharm); IPdR (made by Sparta); CR2412 (made by Cell Therapeutic); L1X (made by Terrapin); or the like. Preferably, the biologically active substance is selected from the group consisting of peptides, poly-peptides, proteins, amino acids, polysaccharides, growth factors, hormones, anti-angiogenesis factors, interferons or cytokines, and pro-drugs. In a particularly preferred embodiment, the biologically active substance is a therapeutic drug or pro-drug, most preferably a drug selected from the group consisting of chemotherapeutic agents and other anti-neoplastics such as paclitaxel, antibiotics, anti-virals, antifungals, anti-inflammatories, and anticoagulants.

The biologically active substances are used in amounts that are therapeutically effective. While the effective amount of a biologically active substance will depend on the particular material being used, amounts of the biologically active substance from about 1% to about 65% may be desirable. Lesser amounts may be used to achieve efficacious levels of treatment for certain biologically active substances.

### Selected Examples of Other Additives

The masking and covering compositions of the invention may also be mixed with natural polymers such as collagen, hyaluronic acid, gelatin, heparin, fibrin and/or heparin sulfate. In certain instances, a synthetic or natural polymer which may or may not be involved in the crosslinking reaction is added either before, during, and/or after mixing of the polalkyleneimine and the polymerization agent. The synthetic or natural polymers can enhance the mechanical properties, affect adhesion, alter the degradation rates, alter viscosity, and/or provide signaling to specific cells. Representative examples of natural polymers which can be added to the masking material and/or covering material include collagen, hyaluronic acid, albumin, cellulose, elastin, fibrin, fibronectin, polylysine, and RGD containing peptides. Examples of synthetic polymers include poly(vinyl acetate), polyvinylpyrrolidone, poly(acrylic acid), poly(ethylene glycol), poly(propylene glycol)-poly(ethylene glycol) copolymer, and trimethylene carbonate. The synthetic or natural polymers to be added can be soluble in aqueous solution or can be insoluble in aqueous solution and dispersed throughout the masking material and/or covering material to create a composite material.

In certain instances, a polyalkylene glycol containing nucleophilic groups is added to the polyalkyleneimine prior to mixing the polyalkyleneimine with a polyalkylene glycol containing electrophilic groups. In certain instances, a PEG is modified to contain amine groups and/or thiol groups. The modified PEG is mixed with the polyalkyleneimine, and then the polyalkyleneimine/modified-PEG solution is added to the PEG-electrophile solution to form the hydrogel. Incorporation of this third active component into the hydrogel can affect hydrogel properties. For example, the resultant hydrogel may swell more, be less mechanically strong, and/or degrade faster compared to a hydrogel prepared without a PEG containing nucleophilic groups.

In certain instances, a polyalkylene glycol containing nucleophilic groups is added to the polyalkyleneimine containing electrophilic groups. In certain instances, the polyalkylene glycol contains amino and/or thiol groups. In certain instances, the polyalkyleneimine contains an N-hydroxysuccinimide group optionally substituted with a sulfonic acid group.

In certain instances, the hydrogel formed by reaction of a polalkyleneimine and a polymerization agent is treated with an acrylate to form a photo-polymerization agent. Then, the photo-polymerization agent is treated with visible or ultra-violet light sufficient to polymerize the photo-polymerization agent.

In certain instances, a polyalkyleneimine containing a plurality of photopolymerizable groups, optionally in the presence of a polyalkylene glycol containing a plurality of photopolymerizable groups, is treated with visible light or ultraviolent light sufficient to polyermize the polalkyleneimine. In certain instances, the photopolymerizable group is an acrylate, such as methacrylate. In certain instances when visible light is used to polymerize the polyalkyleneimine, a photoinitiator is admixed with the polyalkyleneimine. A large number of photoinitiators are known in the art and are amenable to the present invention. For example, eosin y is a photoinitiator that may be used with the polalkyleneimines described herein.

### Sterilization Procedures

A variety of procedures are known in the art for sterilizing a chemical composition. Sterilization may be accomplished by chemical, physical, or irradiation techniques. Examples of chemical methods include exposure to ethylene oxide or hydrogen peroxide vapor. Examples of physical methods include sterilization by heat (dry or moist), retort canning, and filtration. The British Pharmacopoeia recommends heating at a minimum of 160° C. for not less than 2 hours, a minimum of 170° C. for not less than 1 hour and a minimum of 180° C. for not less than 30 minutes for effective sterilization. For examples of heat sterilization, see U.S. Pat. No. 6,136,326, which is hereby incorporated by reference. Passing the chemical composition through a membrane can be used to sterilize a composition. For example, the composition is filtered through a small pore filter such as a 0.22 micron filter which comprises material inert to the composition being filtered. In certain instances, the filtration is conducted in a Class 100,000 or better clean room. Examples of irradiation methods include gamma irradiation, electron beam irradiation, microwave irradiation, and irradiation using visible light. One preferred method is electron beam irradiation, as described in U.S. Pat. Nos. 6,743,858; 6,248,800; and 6,143,805, each of which is hereby incorporated by reference.

There are several sources for electron beam irradiation. The two main groups of electron beam accelerators are: (1) a Dynamitron, which uses an insulated core transformer, and (2) radio frequency (RF) linear accelerators (linacs). The Dynamitron is a particle accelerator (4.5 MeV) designed to impart energy to electrons. The high energy electrons are generated and accelerated by the electrostatic fields of the accelerator electrodes arranged within the length of the glass-insulated beam tube (acceleration tube). These electrons, traveling through an extension of the evacuation beam tube and beam transport (drift pipe) are subjected to a magnet deflection system in order to produce a “scanned” beam, prior to leaving the vacuum enclosure through a beam window. The dose can be adjusted with the control of the percent scan, the beam current, and the conveyor speed. In certain instances, the electron-beam radiation employed may be maintained at an initial fluence of at least about 2 μCurie/cm², at least about 5 μCurie/cm², at least about 8 μCurie/cm², or at least about 10 μCurie/cm². In certain instances, the electron-beam radiation employed has an initial fluence of from about 2 to about 25 μCurie/cm². In certain instances, the electron-beam dosage is from about 5 to 50 kGray, or from about 15 to about 20 kGray with the specific dosage being selected relative to the density of material being subjected to electron-beam radiation as well as the amount of bioburden estimated to be therein. Such factors are well within the skill of the art.

The composition to be sterilized may be in any type of at least partially electron beam permeable container such as glass or plastic. In embodiments of the present invention, the container may be sealed or have an opening. Examples of glass containers include ampules, vials, syringes, pipettes, applicators, and the like. The penetration of electron beam irradiation is a function of the packaging. If there is not enough penetration from the side of a stationary electron beam, the container may be flipped or rotated to achieve adequate penetration. Alternatively, the electron beam source can be moved about a stationary package. In order to determine the dose distribution and dose penetration in product load, a dose map can be performed. This will identify the minimum and maximum dose zone within a product.

Procedures for sterilization using visible light are described in U.S. Pat. No. 6,579,916, which is hereby incorporated by reference. The visible light for sterilization can be generated using any conventional generator of sufficient power and breadth of wavelength to effect sterilization. Generators are commercially available under the tradename PureBright® in-line sterilization systems from PurePulse Technologies, Inc. 4241 Ponderosa Ave, San Diego, Calif. 92123, USA. The PureBright® in-line sterilization system employs visible light to sterilize clear liquids at an intensity approximately 90000 times greater than surface sunlight. If the amount of UV light penetration is of concern, conventional UV absorbing materials can be used to filter out the UV light.

In a preferred embodiment, the composition is sterilized to provide a Sterility Assurance Level (SAL) of at least about 10⁻³. The Sterility Assurance Level measurement standard is described, for example, in ISO/CD 14937, the entire disclosure of which is incorporated herein by reference. In certain embodiments, the Sterility Assurance Level may be at least about 10⁻⁴, at least about 10⁻⁵, or at least about 10⁻⁶.

As discussed above, in certain embodiments of the present invention, one or more of the compositions, reagents, or components of a kit has been sterilized. The sterilization may be achieved using gamma radiation, e-beam radiation, dry heat sterilization, ethylene oxide sterilization, or a combination of any of them. The compositions, reagents, or components of the kits can be sterilized in an aqueous solution or neat.

In certain embodiments a compound of the invention (e.g., a compound of formula Ia, formula Ib, or formula III, as described herein) has been sterilized by e-beam radiation between 2 and 40 kGy.

In certain embodiments a compound of the invention (e.g., a compound of formula Ia, formula Ib, or formula III, as described herein) has been sterilized by e-beam radiation between 3-20 kGy.

In certain embodiments a compound of the invention (e.g., a compound of formula Ia, formula Ib, or formula III, as described herein) has been sterilized by e-beam radiation between 5-12 kGy.

In certain embodiments a compound of the invention (e.g., a compound of formula Ia, formula Ib, or formula III, as described herein) has been diluted in aqueous solution, optionally buffered; and said aqueous solution has been sterilized by e-beam radiation between 2 and 40 kGy.

In certain embodiments a compound of the invention (e.g., a compound of formula Ia, formula Ib, or formula III, as described herein) has been diluted in aqueous solution, optionally buffered; and said aqueous solution has been sterilized by e-beam radiation between 3-20 kGy.

In certain embodiments a compound of the invention (e.g., a compound of formula Ia, formula Ib, or formula III, as described herein) has been diluted in aqueous solution, optionally buffered; and said aqueous solution has been sterilized by e-beam radiation between 5-12 kGy.

In certain embodiments a compound of the invention (e.g., a compound of formula Ia, formula Ib, or formula III, as described herein) has been sterilized with e-beam radiation. In certain embodiments, said e-beam radiation is between 2 and 40 kGy. In certain embodiments, said e-beam radiation is between 3 and 20 kGy. In certain embodiments, said e-beam radiation is between 5 and 12 kGy. In certain embodiments, said sterilization is carried out below 30° C. In certain embodiments, said sterilization is carried out below 20° C. In certain embodiments, said sterilization is carried out below 10° C. In certain embodiments, said sterilization is carried out below 0° C.

In certain embodiments, the present invention relates to the aforementioned method, further comprising the step of sterilizing said polymerization agent.

In certain embodiments, the present invention relates to the aforementioned method, further comprising the step of sterilizing said compound of formula III.

In certain embodiments, the present invention relates to the aforementioned method, wherein said sterilizing is performed by treatment with ethylene oxide, hydrogen peroxide, heat, gamma irradiation, electron beam irradiation, microwave irradiation, or visible light irradiation.

In certain embodiments, the present invention relates to the aforementioned method, wherein said polymerization agent and said compound of formula III have a sterility assurance level of at least about 10⁻³.

In certain embodiments, the present invention relates to the aforementioned method, wherein said polymerization agent and said compound of formula III have a sterility assurance level of at least about 10⁻⁶.

In certain embodiments, the present invention relates to any one of the aforementioned methods, further comprising the step of dissolving in an optionally buffered sterile aqueous solution a compound of formula Ia, Ib, or III to produce a mixture.

In certain embodiments, the present invention relates to any one of the aforementioned methods, further comprising the step of dissolving in an optionally buffered sterile aqueous solution a polymer having one or more monomeric units represented by formula Ie.

In certain embodiments, the present invention relates to the aforementioned method, further comprising the step of sterilizing said mixture using e-beam radiation; and wherein said e-beam radiation is from about 2-40 kGy.

In certain embodiments, the present invention relates to the aforementioned method, further comprising the step of sterilizing said mixture using e-beam radiation; and wherein said e-beam radiation is from about 3-20 kGy.

In certain embodiments, the present invention relates to the aforementioned method, further comprising the step of sterilizing said mixture using e-beam radiation; and wherein said e-beam radiation is from about 5-12 kGy.

In certain embodiments, the present invention relates to any one of the aforementioned methods, further comprising the step of dissolving in an optionally buffered sterile aqueous solution an antioxidant, and a compound of formula Ia, Ib, or III.

In certain embodiments, the present invention relates to any one of the aforementioned methods, further comprising the step of dissolving in an optionally buffered sterile aqueous solution an antioxidant, and a polymer having one or more monomeric units represented by formula Ie.

In certain embodiments, the present invention relates to the aforementioned method, further comprising the step of sterilizing said mixture using e-beam radiation; and wherein said e-beam radiation is from about 2-40 kGy.

In certain embodiments, the present invention relates to the aforementioned method, further comprising the step of sterilizing said mixture using e-beam radiation; and wherein said e-beam radiation is from about 3-20 kGy.

In certain embodiments, the present invention relates to the aforementioned method, further comprising the step of sterilizing said mixture using e-beam radiation; and wherein said e-beam radiation is from about 5-12 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 50 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 50 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 25 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 25 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 10 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 10 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 5 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 5 wt % of said mixture; and wherein said e-beam radiation is from about 2-40 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 50 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 50 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 25 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 25 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 10 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 10 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 5 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 5 wt % of said mixture; and wherein said e-beam radiation is from about 3-20 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 50 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 50 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 25 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 25 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 10 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 10 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a compound of formula Ia or Ib to produce a mixture; and sterilizing said mixture using e-beam radiation; wherein said compound of formula Ia or Ib constitutes from about 0.01 wt % to about 5 wt % of said mixture; and wherein said e-beam radiation is from about 5-12 kGy.

In certain embodiments, the present invention relates to any one of the aforementioned methods, further comprising the steps of dissolving in an optionally buffered sterile aqueous solution a polymer having one or more monomeric units represented by formula Ie to produce a mixture; and sterilizing said mixture using e-beam radiation; wherein said polymer constitutes from about 0.01 wt % to about 5 wt % of said mixture; and wherein said e-beam radiation is from about 5-12 kGy.

### Delivery Systems

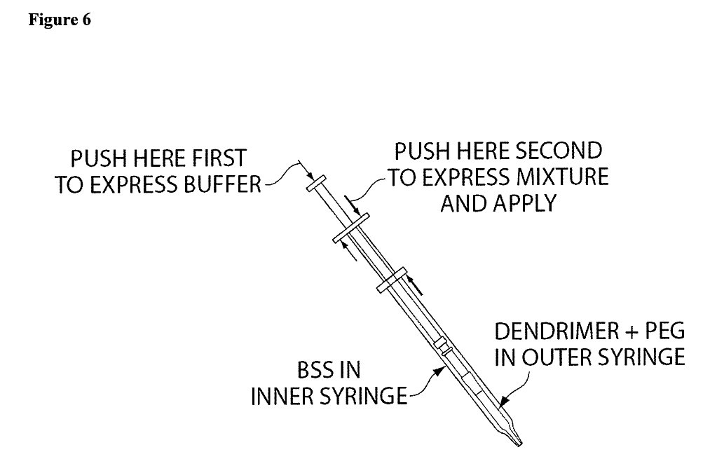

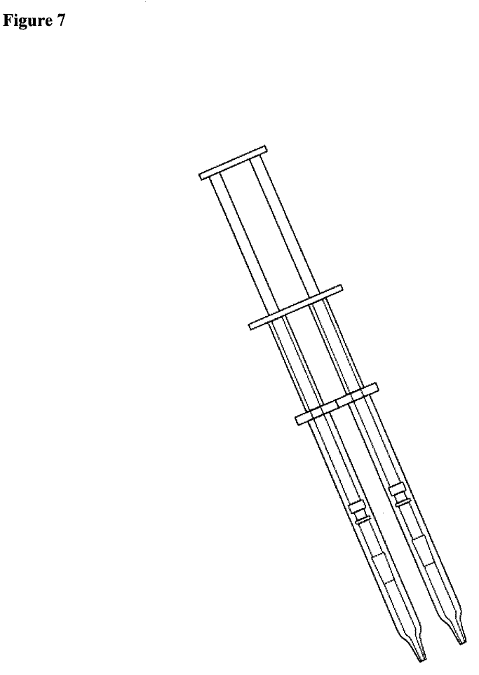

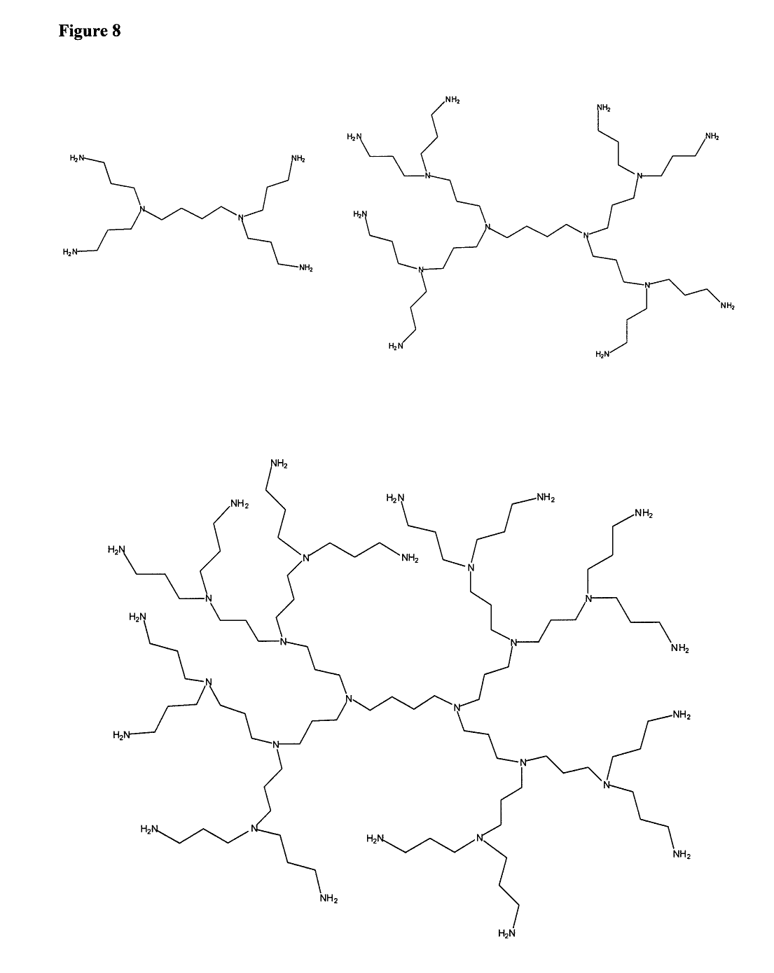

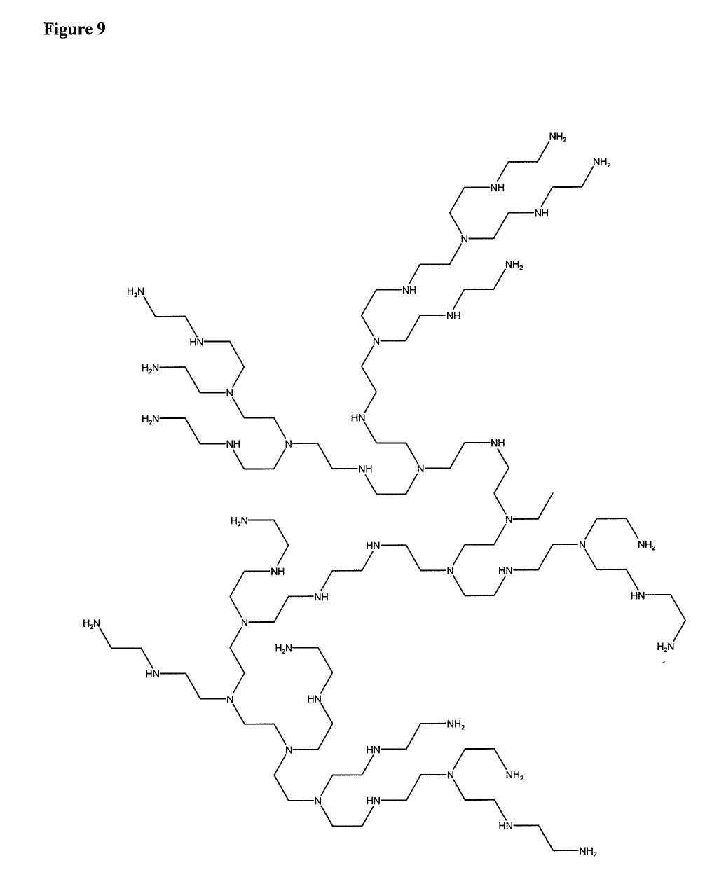

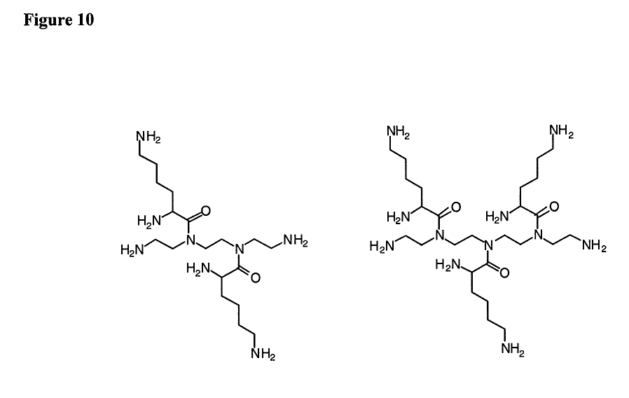

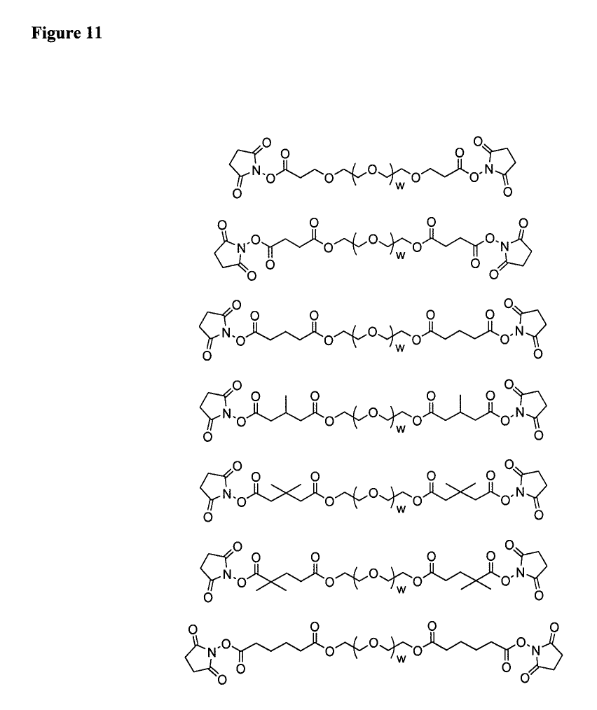

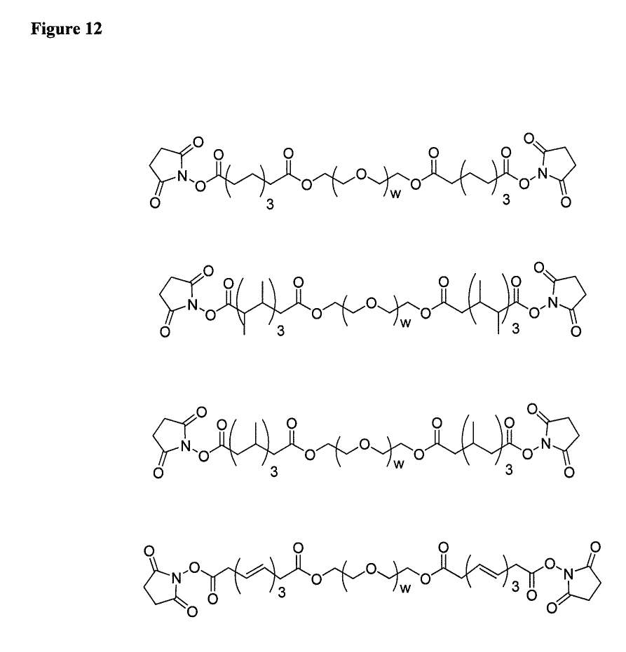

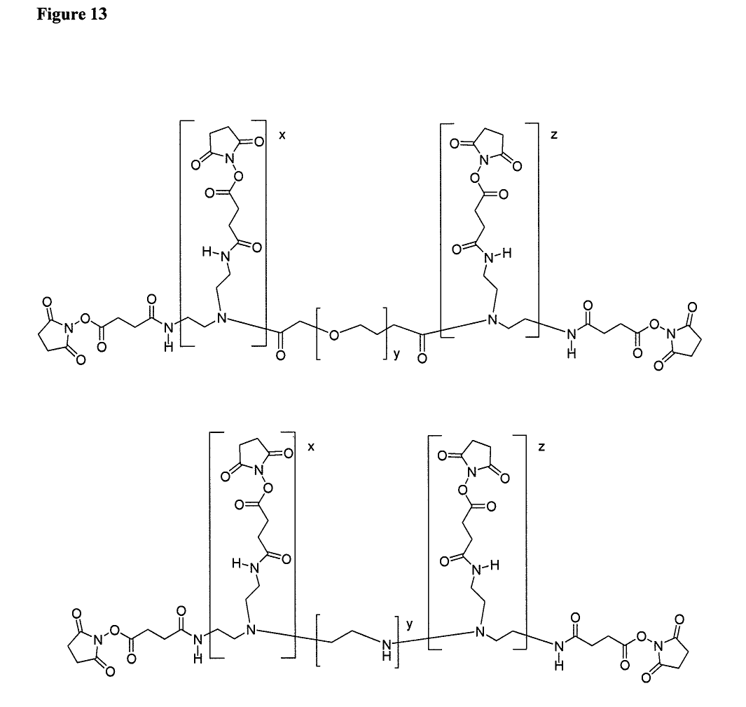

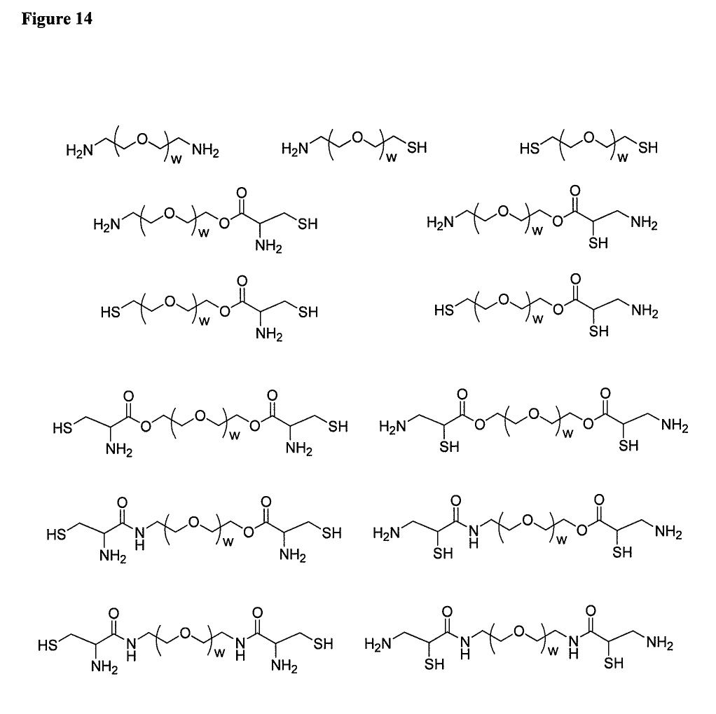

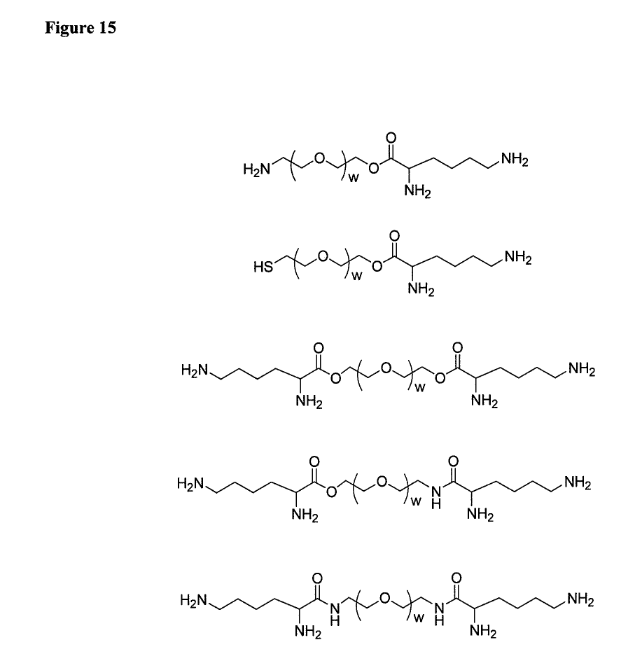

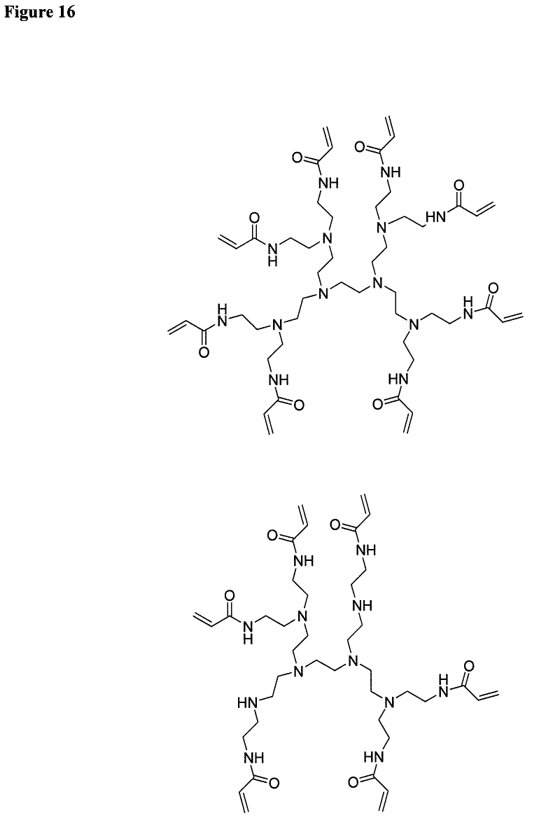

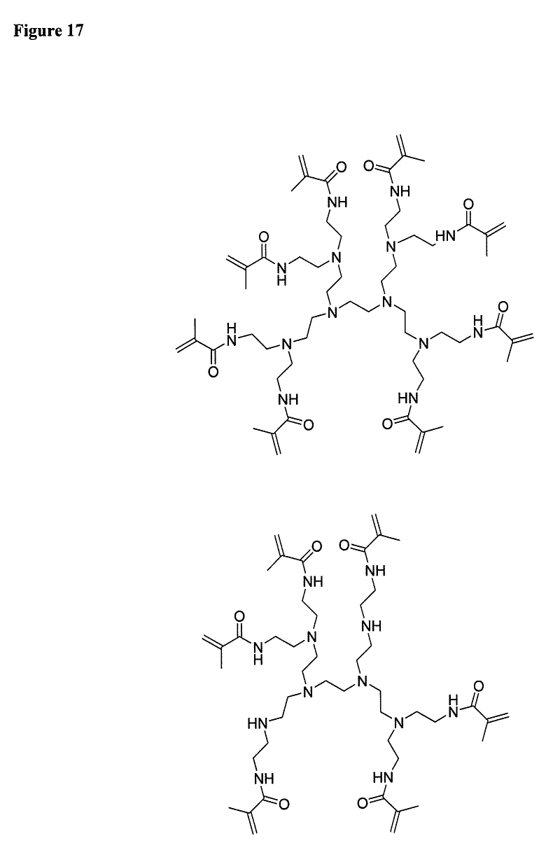

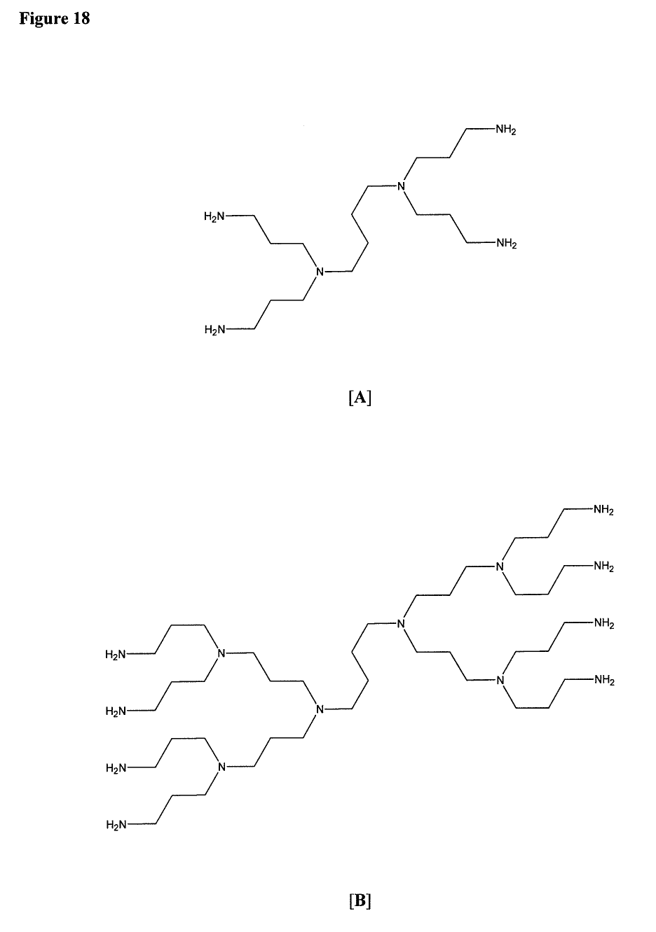

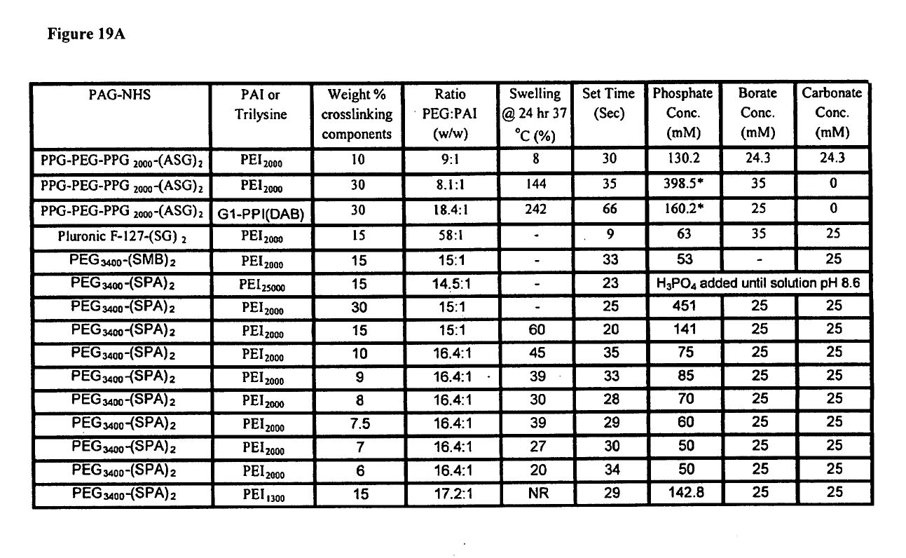

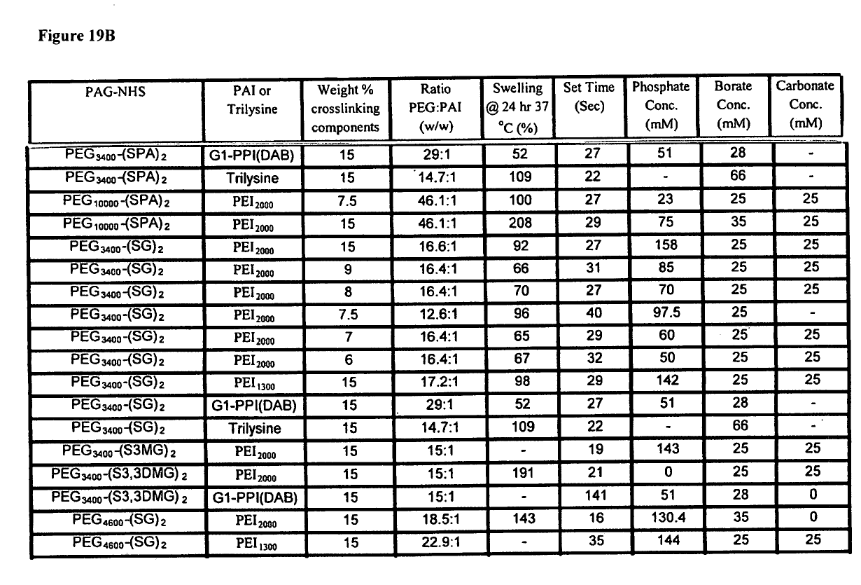

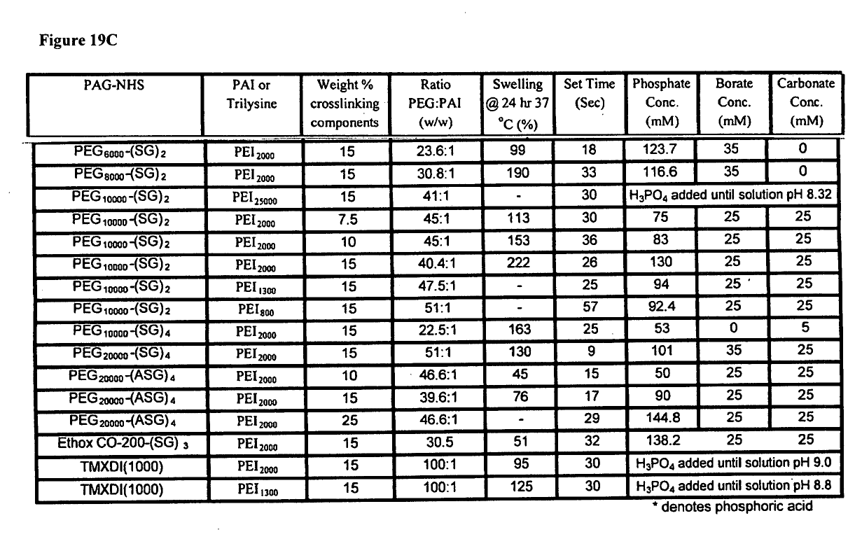

The materials used to form the masking material or the covering material of the present invention may be delivered to the wound, void, or damaged tissue of a patient using a large number of known delivery devices. For example, the delivery system may be a single-barrel syringe system. In certain instances, the single-barrel syringe is a double acting, single-barrel syringe system as displayed in FIG. 6. In certain situations, a double- or multi-barrel syringe system, as displayed in FIG. 7, may be preferable. In instances where the polymerizable polyalkyleneimine is mixed with a polymerization agent prior to delivering the solution to the wound, void, or damaged tissue of a patient, a delivery device that flows two or more streams of liquid in a mixing chamber may be preferable. Alternatively, a delivery device that mixes two solids and two liquids and then separately flows these streams of liquid to a mixing chamber may be advantageous. In certain instances, delivery may be assisted with machines, compressed air or gases, and the like. Of course, variations may be made in the size of the delivery device, the length of the delivery device, and/or the use of machines to aid in delivery.

In certain instances, a delivery system is used to deliver the materials to the wound, void, or damaged tissue of a patient, wherein at least two dry, reactive components are stored together in a dry state and introduced into a liquid component(s) at the time of use to form a mixture that forms a hydrogel.

In certain instances, it may be advantageous the mix the components used to form the hydrogel by static mixing device such as a tortuous path mixing element. As an example, both components could be dissolved in aqueous solution prior to use. Once mixed, the solutions would polymerize in a predetermined amount of time.

Another aspect of the invention relates to a method of preparing a hydrogel, comprising the steps of combining an aqueous solution of a first component, and a neat form of a second component to give a mixture; and applying the mixture to a tissue site. In certain embodiments, the present invention relates to the aforementioned method, wherein said step of combining to give said mixture occurs shortly before said step of applying. In certain embodiments, the present invention relates to the aforementioned method, wherein said step of combining to give said mixture occurs less than about 30 minutes before said step of applying. In certain embodiments, the present invention relates to the aforementioned method, wherein said step of combining to give said mixture occurs less than about 20 minutes before said step of applying. In certain embodiments, the present invention relates to the aforementioned method, wherein said step of combining to give said mixture occurs less than about 10 minutes before said step of applying. In certain embodiments, the present invention relates to the aforementioned method, wherein said step of combining to give said mixture occurs less than about 5 minutes before said step of applying.

Another aspect of the invention relates to a method of controlling the polymerization of a two component hydrogel system through combining the two components in an aqueous solution in one container with a final solution pH in a range unsuitable for crosslinking, and expressing the solution through an ion exchange resin to either lower or raise the pH of the solution to a range suitable for crosslinking. For example, the two components could be mixed (without gelation) prior to applying the mixture to a patient. The pH of the mixing solution may be adjusted in order to slow or prevent crosslinking of hydrogel components. Once the components used to form the hydrogel are mixed, the resultant solution may be contacted with a fit or resin designed to raise or lower the pH to a level suitable for crosslinking.

Another aspect of the invention relates to a method of controlling the polymerization of a two component hydrogel system through combining an aqueous solution of the first component with a neat form of the second component with a final solution pH in a range unsuitable for crosslinking, and expressing the solution through an ion exchange resin to either lower or raise the pH of the solution to a range suitable for crosslinking.

For example, PEG-NHS and a PEI could be mixed during packaging and dissolved prior to use in a buffer designed to provide a solution with a pH of about 6. The solution is mixed, and then the solution is contacted with a resin embedded in the delivery device. The resin would raise the pH to about 7 or 8 for initiate crosslinking.

Another aspect of the invention relates to one the methods described herein for sealing a wound, void, or damaged tissue wherein the components are PEG-NHS and PEI Mw2000, the initial pH of the solution containing the combined components is below approximately pH 7, and the ion exchange resin is an anion exchange resin (including but not limited to MTO-Dowex M43, Dowex 66, or Dowex 1×2-200).

Another aspect of the invention relates to a method of controlling the polymerization of a two component hydrogel system through combining the two components in an aqueous solution in one container with a final solution pH in a range unsuitable for crosslinking, and expressing the solution through an frit/resin coated/loaded with an acidic or basic media to lower or raise the pH of the solution to a range suitable for crosslinking.

Another aspect of the invention relates to a method of controlling the polymerization of a two component hydrogel system through combining the two components in an aqueous solution in one container with a final solution pH in a range unsuitable for crosslinking, and contacting the solution with an applicator loaded with either an acidic or basic media to lower or raise the pH of the solution to a range suitable for crosslinking.

It is appreciated that the above methods may be optimized by modifying, inter alia, the size and shape of the instrument that that delivers the solution suitable for crosslinking. For example, the diameter and/or length of the crosslinking-solution holding chamber can be altered, or the diameter and/or length of the chamber housing the frit/resin loaded with an acidic or basic media can be altered. Similarly, the applicator tip of the delivery instrument can be permanent or disposable. The delivery instrument may be constructed so that the masking material and/or covering material is applied as a spray, mist, or liquid. In certain instances, the delivery instrument is a single or double barrel syringe. Further, it is appreciated that the above methods may involve air-assisted delivery the crosslinking solution. In certain instances, the above methods may employ a brush or sponge to delivery the hydrogel to the tissue.

### Kits of the Invention

Another aspect of the present invention relates to a kit for the preparation of a first material and/or a second material comprising: a hydrogel material for use as a masking material; and instructions describing the uses of a hydrogel as a temporary patch, the use of a preformed hydrogels, which can have a wide range of degradation rates, the use of a dissolvable film, or the use of a more permanent patch, such as an existing co-masking material (e.g., commercial dural patch materials).

In certain embodiments, the kit would also contain a device to deliver the material to the surgical site, if one is necessary for the specific material.

Another aspect of the present invention relates to a kit, comprising: a hydrogel outer layer (covering layer), which will cover the anti-adhesion layer (masking material); and a device to deliver the hydrogel to the surgical site.

In certain embodiments, the kit would be sterilized, either together or as separate components, prior to final assembly of the kit.

Another aspect of the present invention relates to a kit, comprising: a current duraplasty material and an in situ polymeric sealant.

Another aspect of the present invention relates to a kit for the preparation of a first material and/or a second material comprising: a polymerization agent selected from the group consisting of a compound of formula Ia or formula Ib, wherein formulae Ia or Ib are as defined above; and instructions for preparing said gel.

Another aspect of the present invention relates to a kit for the preparation of a first material and/or a second material comprising: a polymerization agent selected from the group consisting of a compound of formula Ia, formula Ib, and formula Ic, wherein formulae Ia, Ib, and Ic are as defined above; and instructions for preparing said gel

Another aspect of the present invention relates to a kit for the preparation of a first material and/or a second material comprising: a compound of formula I and formula III, wherein formulae I and III are as defined above; and instructions for preparing said gel.

Another aspect of the present invention relates to a kit for the preparation a first material and/or a second material comprising: a compound of formula Ic, wherein formula Ic is as defined above; and instructions for preparing said gel.

In certain embodiments, the present invention relates to the aforementioned kit, further comprising a compound of formula III, wherein formula III is as defined above.

In certain embodiments, the present invention relates to the aforementioned kit, further comprising a desiccant.

In certain embodiments, the present invention relates to the aforementioned kit, further comprising an antioxidant.

In certain embodiments, the present invention relates to the aforementioned kit, wherein said antioxidant is selected from the group consisting of sodium metabisulfite, citric acid, and ascorbic acid.

In certain embodiments, the present invention relates to the aforementioned kit, further comprising an inert atmosphere.

In certain embodiments, the present invention relates to the aforementioned kit, wherein said kit has a sterility assurance level of at least about 10⁻³.

In certain embodiments, the present invention relates to the aforementioned kit, wherein said kit has a sterility assurance level of at least about 10⁻⁶.

In certain embodiments, the present invention relates to the aforementioned kit, wherein said kit was sterilized using E-beam or gamma radiation.

In certain embodiments, the present invention relates to the aforementioned kit, wherein said kit was sterilized using E-beam radiation.

In certain embodiments, the present invention relates to the aforementioned kit, wherein said e-beam radiation is between 2 and 100 kGy.

In certain embodiments, the present invention relates to the aforementioned kit, wherein said e-beam radiation is between 10 and 80 kGy.

In certain embodiments, the present invention relates to the aforementioned kit, wherein said e-beam radiation is between 15 and 40 kGy.

In certain embodiments, the present invention relates to the aforementioned kit, wherein said e-beam radiation is between 2 and 40 kGy.

In certain embodiments, the present invention relates to the aforementioned kit, wherein said e-beam radiation is between 3 and 20 kGy.

In certain embodiments, the present invention relates to the aforementioned kit, wherein said e-beam radiation is between 5 and 12 kGy.

In certain embodiments, the present invention relates to the aforementioned kit, wherein said kit was sterilized by multiple exposures to E-beam or gamma radiation.

In certain embodiments, the present invention relates to the aforementioned kit, wherein said kit comprises more than one compound of formula III.

In certain embodiments, the present invention relates to the aforementioned kit, wherein said kit comprises more than one compound of formula Ia or Ib.

In certain embodiments, the present invention relates to the aforementioned kit, wherein said kit further comprises a medicament, colorant, flavoring, scent, fibrous additive, thickener or plasticizer.

In certain embodiments, the present invention relates to the aforementioned kit, further comprising a moisture-barrier element. The moisture-barrier element may be conditioned for use in the preparation of a solution to be used in a method according to the present invention. Alternatively, a second component of the kit may be contained within the moisture-barrier element. For example, a water-sensitive reagent, such as a PEG-bis(NHS ester), may be contained in a moisture-barrier element, thereby limiting or preventing hydrolysis of the water-sensitive reagent between the manufacture date and the use date of the kit. Further, a kit may contain a plurality of moisture-barrier elements, each of which may be conditioned for use in the same or distinct ways. For example, for a kit containing a plurality of water-sensitive reagents each of them may be contained in an individual moisture-barrier element. Alternatively, a moisture-barrier element may contain a plurality of water-sensitive reagents.

A moisture-barrier element may be characterized in a number of ways or a combination of them. For example, a moisture-barrier element may be characterized by its shape (e.g., pouch, vial, sachet, ampule); composition (e.g., glass, foil, Teflon®, stainless steel); and/or it may be characterized by a functional quality (e.g., moisture-vapor transmission rate (MVTR)). MVTR is an important means of characterizing a moisture-barrier element because: those of ordinary skill in the art understand how to measure the MVTR of a material; MVTR values for various materials are known; and the MVTR of a moisture-barrier element quantifies its ability to exclude water from it contents.

In certain embodiments, the present invention relates to the aforementioned kit, further comprising a moisture-barrier element with a moisture vapor transmission rate (MVTR) less than or equal to about 0.15 gram per 100 square inches per day.

In certain embodiments, the present invention relates to the aforementioned kit, further comprising a moisture-barrier element with a moisture vapor transmission rate (MVTR) less than or equal to about 0.02 gram per 100 square inches per day.

In certain embodiments, the present invention relates to the aforementioned kit, further comprising a moisture-barrier element with a moisture vapor transmission rate (MVTR) less than or equal to about 0.15 gram per 100 square inches per day; wherein said moisture-barrier element comprises said polymerization agent selected from the group consisting of a compound of formula Ia and formula Ib.

In certain embodiments, the present invention relates to the aforementioned kit, further comprising a moisture-barrier element with a moisture vapor transmission rate (MVTR) less than or equal to about 0.02 gram per 100 square inches per day; wherein said moisture-barrier element comprises said polymerization agent selected from the group consisting of a compound of formula Ia and formula Ib.

In certain embodiments, the present invention relates to the aforementioned kit, further comprising a catheter.

In certain embodiments, the present invention relates to the aforementioned kit, further comprising a syringe.

In certain embodiments, the present invention relates to the aforementioned kit, further comprising a brush.

In certain embodiments, the present invention relates to the aforementioned kit, further comprising a spray container and/or an aerosol container.

In certain embodiments, the present invention relates to the aforementioned kit, further comprising a device for endoscopic delivery. Endoscopy is a surgical technique that involves the use of an endoscope, a special viewing instrument that allows a surgeon to see images of the body's internal structures through very small incisions. Endoscopic surgery has been used for decades in a number of different procedures, including gallbladder removal, tubal ligation, and knee surgery. An endoscope typically consists of two basic parts: A tubular probe fitted with a tiny camera and bright light, which is inserted through a small incision; and a viewing screen, which magnifies the transmitted images of the body's internal structures. During surgery, the surgeon watches the screen while moving the tube of the endoscope through the surgical area.

In certain embodiments, the present invention relates to the aforementioned kit, further comprising a device for laparoscopic delivery. Laparoscopic surgery is a “minimally invasive” surgical technique. Laparoscopy has been used successfully to treat gynecological problems, gallbladder disease, and perform colorectal surgery for many years. The word “laparoscopy” means to look inside the abdominal cavity with a special camera or “scope.” Laparoscopy, also known as “keyhole” surgery, has also been used for many years to diagnose medical conditions inside the abdominal cavity.

In certain embodiments of the kits, a liquid reagent is contained in a vial, and a powdered reagent is contained in a single-barreled syringe. At time of use, the vial and syringe are placed into liquid communication, and the liquid is withdrawn from the vial into the powder-filled syringe, thereby mixing the two reagents.

In another embodiment, the liquid portion is housed within an outer housing into which at least one hollow, inner piston is placed. The at least one hollowed, inner piston is then filled with the powdered portion of the hydrogel formulation. The at least one hollow, inner piston is designed to exclude the liquid portion until it is manually depressed. When depressed, the bottom of the piston passed through a sealing ring in the outer housing and liquid is allowed to pass into the hollowed center of the at least one piston, thereby contacting and dissolving the powder. The powder is thereby dissolved and optionally mixed using an applicator component, such as a brush, swab or syringe canula. The mixture is then applied to the surface of the tissue to be augmented, sealed or bonded.

In another embodiment, the liquid and powder reagents that produce the hydrogel formulation are sealed within two separate, but adjacent, formed wells of a form/fill/seal pouch or sachet. The seal between the two wells is designed to be frangible. At time of use, the user manually pressurizes the liquid-containing well, thus rupturing the frangible seal and allowing the liquid to flow into the powder-containing well. The mixture can then be mixed with a kneading action and liberated from the form/fill/seal pouch either through another frangible seal, a valve, or by tearing or cutting the pouch or sachet.

In another embodiment, the liquid and solid reagents that produce the hydrogel formulation are separate, where the solid reagent is absorbed to a bush and separated from the liquid. At time of use, the user manually pushes the liquid into the brush where the solid and liquid mix to afford the masking material and/or covering material, which is then subsequently applied.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “nucleophile” is recognized in the art, and as used herein means a chemical moiety having a reactive pair of electrons.

The term “electrophile” is art-recognized and refers to chemical moieties which can accept a pair of electrons from a nucleophile as defined above. Electrophilic moieties useful in the method of the present invention include halides and sulfonates.

The term “tissue plane” refers to a tissue having an exposed surface area.

The term “polymerize” as used herein refers to the process of converting a monomer to a chain of monomers, wherein the chain of monomers comprises at least about 5 monomers. In certain instances, the chain of monomers comprises at least about 10 or 15 monomers. In certain instances, the chain of monomers comprises at least about 25 or 40 monomers. In certain instances, the chain of monomers comprises at least about 50 or 75 monomers. In certain instances, the chain of monomers comprises at least about 100 or 150 monomers. In instances wherein the monomeric unit has more than one functional group capable of forming a bond in the polymerization reaction, the term “polymerize” indicates that at least one of functional groups capable of forming a bond in the polymerization reaction forms a bond with another compound, generally speaking, the other compound is another monomer. In certain instances, at least about 10% of the functional groups capable of forming a bond in a polymerization reaction form a bond to another monomer. In certain instances, at least about 25% of the functional groups capable of forming a bond in a polymerization reaction form a bond to another monomer. In certain instances, at least about 50% of the functional groups capable of forming a bond in a polymerization reaction form a bond to another monomer. In certain instances, at least about 75% of the functional groups capable of forming a bond in a polymerization reaction form a bond to another monomer. In certain instances, about 20% to about 50% of the functional groups capable of forming a bond in a polymerization reaction form a bond to another monomer.

The term “seal” as used herein indicates that a protective barrier is formed over the wound. In certain instances, the protective barrier is a continuous layer. In certain instances, the protective barrier is a discontinuous layer, i.e., a layer that has holes or pores in the layer. In certain instances, the discontinuous layer comprises less than about 25% holes. In certain instances, the discontinuous layer comprises about less than 15% holes. In certain instances, the discontinuous layer comprises about less than 5% holes. In the instance where the protective barrier is a continuous layer, in certain embodiments, certain fluids or gases can penetrate through the layer.

For purposes of this invention, the chemical elements are identified in accordance with the Periodic Table of the Elements, CAS version, Handbook of Chemistry and Physics, 67th Ed., 1986-87, inside cover.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, alkylthio, sulfonyl, sulfonamido, ketone, aldehyde, ester, heterocyclyl, aromatic or heteroaromatic moieties, trifluoroalkyl, cyano, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkylthio, sulfonyl, ketone, aldehyde, ester, a heterocyclyl, an aromatic or heteroaromatic moiety, trifluoroalkyl, cyano, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkylthio, sulfonyl, ketone, aldehyde, ester, a heterocyclyl, an aromatic or heteroaromatic moiety, trifluoroalkyl, cyano, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on page 560 of “Advanced Inorganic Chemistry” by Cotton and Wilkinson, that is, for example, monovalent anionic groups sufficiently electronegative to exhibit a positive Hammett sigma value at least equaling that of a halide (e.g., CN, OCN, SCN, SeCN, TeCN, N₃, and C(CN)₃).

The terms “amine” and “amino” are art-recognized and refer to both unsubstituted and substituted amines, e.g., a moiety that may be represented by the general formulas:

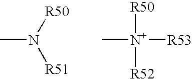

wherein R50, R51, R52 and R53 each independently represent a hydrogen, an alkyl, an alkenyl, —(CH₂)ₘ—R61, or R50 and R51 or R52, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

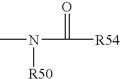

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

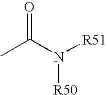

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

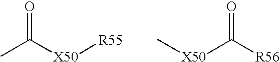

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

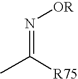

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

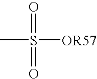

in which R57 is an electron pair, hydrogen, alkyl, cycloalkyl, or aryl.

The term “sulfate” is art recognized and includes a moiety that may be represented by the general formula:

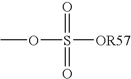

in which R57 is as defined above.

The term “sulfonamido” is art recognized and includes a moiety that may be represented by the general formula:

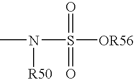

in which R50 and R56 are as defined above.

The term “sulfamoyl” is art-recognized and refers to a moiety that may be represented by the general formula:

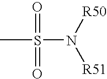

in which R50 and R51 are as defined above.

The term “sulfonyl” is art-recognized and refers to a moiety that may be represented by the general formula:

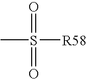

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

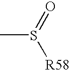

in which R58 is defined above.

The term “phosphoryl” is art-recognized and may in general be represented by the formula:

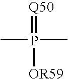

wherein Q50 represents S or O, and R59 represents hydrogen, a lower alkyl or an aryl. When used to substitute, e.g., an alkyl, the phosphoryl group of the phosphorylalkyl may be represented by the general formulas:

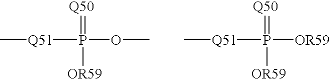

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

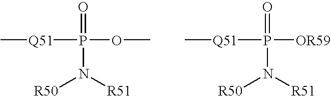

wherein Q51, R50, R51 and R59 are as defined above.

The term “phosphonamidite” is art-recognized and may be represented in the general formulas:

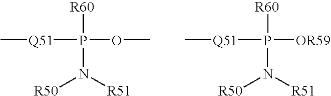

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The term “PEG(NHS)₂” refers to a polyethylene glycol having the following functional group at both ends of the polymer chain:

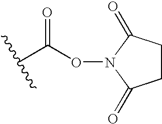

PEG(NHS)₂ can be prepared using either of the following methods. In method 1, a polyethylene glycol is subjected to oxidative conditions in order to oxidize the two termini to the corresponding carboxylic acids [HO₂CCH₂O-PEG-OCH₂CO₂H], followed by transformation to the bis(NHS ester). In method 2, PEG(NHS)₂ is prepared by alkylation of the two termini of a polyethylene glycol with acrylonitrile to give NCCH₂CH₂O-PEG-OCH₂CH₂CN, followed by hydrolysis to the bis(acid) [HO₂CCH₂CH₂O-PEG-OCH₂CH₂CO₂H], and then transformation to the bis(NHS ester).

The term “SS” refers to the following chemical group:

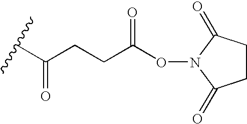

The term “SG” refers to the following chemical group:

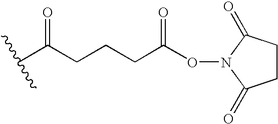

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The definition of each expression, e.g., alkyl, m, n, and the like, when it occurs more than once in any structure, is intended to be independent of its definition elsewhere in the same structure.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

While several embodiments of the present invention are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

### EXEMPLIFICATION

The invention now being generally described, it will be more readily understood by reference to the following examples which are included merely for purposes of illustration of certain aspects and embodiments of the present invention, and are not intended to limit the invention.

### EXAMPLE 1

A piece of oil free collagen (about 4 cm by about 15 cm) was prepared by exhaustively rinsing with acetone and subsequent drying. After cleaning, the collagen was placed into DI water to hydrate just prior to use. An about 1 cm by about 1 cm hole was cut into the collagen such that the collagen sheet could be folded back over upon itself to yield a hole with an underlying collagen substrate. The hole on the top layer would be considered the wound, the area around the hole would be considered the healthy tissue, and the hole area inside the hole would be considered wounded tissue or an underlying substrate to which adhesion to is undesired. 280 mg of PEG-3350 succinimidyl sebacate was weighed into a vial and then dissolved into 680 μL of DI water. A PEI solution was prepared by dissolving 850 mg of PEI 2,000 (Lugalvan G50, 50% solids) and 570 mg of sodium borate into 25 mL of DI water in a 25 mL volumetric flask. 0.85 mL of the PEG solution was charged to a 1 mL syringe and 0.85 mL of the PEI solution was charged to a second syringe. The syringes were then connected to a dual syringe atomizer spray applicator. Just prior to the application of materials, the collagen sheet was laid down onto to a piece of plastic sheet material. A layer of K—Y Liquid was applied to the collagen on the side opposite the hole. The sheet was folded over such that the square hole now laid on top of the section of collagen coated with the K—Y Liquid. At this point the underlying collagen exposed by the hole was coated with K—Y liquid, but none of the collagen top layer around the hole was covered by K—Y Liquid. Just prior to application of the hydrogel, a second coating of K—Y Liquid was applied to the collagen inside the area of the square. The liquids in the dual syringe spray applicator were then expressed out of the device over the hole and surrounding collagen in a smooth sweeping motion. The hydrogel was allowed to cure for about 30-40 seconds. The collagen sheet was then carefully unfolded to yield a sheet of hydrogel which was connected only to the top layer of collagen and fell free from the under lying collagen. A small section of the gel (about 10%) did break free from the main hydrogel during the peeling motion. This section was easily slid across the underlying collagen and was not fixed to the surface. This is an indication that the application of the hydrogel was not completely uniform and led to a weak spot in the gel.

### EXAMPLE 2

A piece of oil free collagen (about 4 cm by about 15 cm) was prepared by exhaustively rinsing with acetone and subsequent drying. After cleaning, the collagen was placed into DI water to hydrate just prior to use. An about 1 cm by about 1 cm hole was cut into the collagen such that the collagen sheet could be folded back over upon itself to yield a hole with an underlying collagen substrate. The hole on the top layer would be considered the wound, the area around the hole would be considered the healthy tissue, and the hole area inside the hole would be considered wounded tissue or an underlying substrate which adhesion to is undesired. 280 mg of PEG-3350 succinimidyl sebacate was weighed into a vial and then dissolved into 680 μL of DI water. A PEI solution was prepared by dissolving 850 mg of PEI 2,000 (Lugalvan G50, 50% solids) and 570 mg of sodium borate into 25 mL of DI water in a 25 mL volumetric flask. 0.85 mL of the PEG solution was charged to a 1 mL syringe and 0.85 mL of the PEI solution was charged to a second syringe. The syringes were then connected to a dual syringe atomizer spray applicator. Just prior to the application of materials, the collagen sheet was laid down onto to a piece of plastic sheet material. A layer of K—Y Jelly was applied to the collagen on the side opposite the hole. The sheet was folded over such that the square hole now laid on top of the section of collagen coated with the K—Y Jelly. At this point the underlying collagen exposed by the hole was coated with K—Y Jelly, but none of the collagen top layer around the hole was covered by K—Y Jelly. Just prior to application of the hydrogel, a second coating of K—Y Jelly was applied to the collagen inside the area of the square. The liquids in the dual syringe spray applicator were then expressed out of the device over the hole and surrounding collagen in a smooth sweeping motion. The hydrogel was allowed to cure for 30-40 seconds. The collagen sheet was then carefully unfolded to yield a sheet of hydrogel which was connected only to the top layer of collagen and fell free from the under lying collagen. In this case, a completely intact hydrogel lifted off the exposed under layer of collagen.

### EXAMPLE 3

A piece of oil free collagen (about 4 cm by about 15 cm) was prepared by exhaustively rinsing with acetone and subsequent drying. After cleaning, the collagen was placed into DI water to hydrate just prior to use. An about 1 cm by about 1 cm hole was cut into the collagen such that the collagen sheet could be folded back over upon itself to yield a hole with an underlying collagen substrate. The hole on the top layer would be considered the wound, the area around the hole would be considered the healthy tissue, and the hole area inside the hole would be considered wounded tissue or an underlying substrate which adhesion to is undesired. 287 mg of PEG-3350 succinyl succinate was weighed into a vial and then dissolved into 690 μL of DI water. This solution was brought up into a 1 mL syringe. A PEI solution was prepared by dissolving 839 mg of PEI 2,000 (Lugalvan G50, 50% solids) and 570 mg of sodium borate into 25 mL of DI water in a 25 mL volumetric flask. 980 μL of the PEI solution was charged to a second 1 ml syringe. The two syringes were then connected to a dual syringe spray applicator. The collagen sheet was then laid down onto a piece of plastic. The succinyl succinate gel was then applied to the side adjacent to the square hole. The hydrogel was allowed to set up for several minutes. During this time the collagen was covered with plastic to prevent dehydration. 280 mg of PEG-3350 Succinimidyl propionic acid (SPA) was weighed into a vial and then dissolved into 680 μL of DI water. A PEI solution was prepared by dissolving 850 mg of PEI 2,000 (Lugalvan G50, 50% solids) and 570 mg of sodium borate into 25 mL of DI water in a 25 mL volumetric flask. 960 μL of the PEG solution was charged to a 1 mL syringe and 960 μL of the PEI solution was charged to a second syringe. The syringes were then connected to a dual syringe atomizer spray applicator. The plastic sheet was removed from the collagen sheet. The sheet was then folded in half such that section of collagen with the hole covered the section sprayed with the first hydrogel. The top layer of collagen was pressed down to make sure that the edges of the hole were flat against the underlying surface. The PEG-SPA hydrogel was then sprayed over the top surface to create a uniform layer of hydrogel over the hole and extending onto the collagen. The gel was allowed to set up for 5 minutes. At this point the top hydrogel was adhered to the underlying hydrogel. The collagen sheet was then placed into a pH 7.4 PBS solution in a plastic jar. The jar was capped and then placed into a 37° C. oven and allowed to stand for 1.5 h. Upon removal from the PBS solution, the SPA hydrogel was shown to have delaminated from the underlying surface, which indicated that the SS gel had decomposed. The SPA hydrogel was a complete gel which spanned the 1 cm by 1 cm hole.

### EXAMPLE 4

A piece of oil free collagen (about 4 cm by about 15 cm) was prepared by exhaustively rinsing with acetone and subsequent drying. After cleaning, the collagen was placed into DI water to hydrate just prior to use. An about 1 cm by about 1 cm hole was cut into the collagen such that the collagen sheet could be folded back over upon itself to yield a hole with an underlying collagen substrate. The hole on the top layer would be considered the wound, the area around the hole would be considered the healthy tissue, and the hole area inside the hole would be considered wounded tissue or an underlying substrate which adhesion to is undesired. 287 mg of PEG-3350 succinyl succinate was weighed into a vial and then dissolved into 690 μL of DI water. This solution was brought up into a 1 mL syringe. A PEI solution was prepared by dissolving 839 mg of PEI 2,000 (Lugalvan G50, 50% solids) and 570 mg of sodium borate into 25 mL of DI water in a 25 mL volumetric flask. 980 μL of the PEI solution was charged to a second 1 ml syringe. The two syringes were then connected to a dual syringe spray applicator. The collagen sheet was then laid down onto a piece of plastic. The PEG succinimidyl succinate gel was then applied to the side adjacent to the square hole. The hydrogel was allowed to set up for several minutes. During this time the collagen was covered with plastic to prevent dehydration. 295 mg of PEG-3350-SPA was weighed into a vial and then dissolved into 710 μL of DI water. A PEI solution was prepared by dissolving 850 mg of PEI 2,000 (Lugalvan G50, 50% solids) and 570 mg of sodium borate into 25 mL of DI water in a 25 mL volumetric flask. The PEG-succinimidyl succinate solution was charged to a 1 mL syringe and 1001 μL of the PEI solution was charged to a second syringe. The syringes were then connected to a dual syringe atomizer spray applicator. The plastic sheet was removed from the collagen sheet. The sheet was then folded in half such that section of collagen with the hole covered the section sprayed with the first hydrogel. The top layer of collagen was pressed down to make sure that the edges of the hole were flat against the underlying surface. The PEG-SPA hydrogel was then sprayed over the top surface to create a uniform layer of hydrogel over the hole and extending onto the collagen. The gel was allowed to set up for 5 minutes. At this point the top hydrogel was adhered to the underlying hydrogel. The collagen sheet was then placed into a pH 7.4 PBS solution in a plastic jar. The jar was capped and then placed into a 37° C. oven and allowed to stand for 1.5 h. Upon removal from the PBS solution, the SPA hydrogel was shown to have delaminated from the underlying surface, which indicated that the SS gel had decomposed. The SPA hydrogel was a complete gel which spanned the 1 cm by 1 cm hole.

### EXAMPLE 5

A piece of collagen was prepared as in Example 4. In the process of preparing the top layer, the PEG succinimidyl succinate hydrogel, in Example 4, the gel was sprayed onto the plastic which the collagen had been placed. A portion of this gel was removed as a sheet from the plastic. This piece of gel was cut to yield a piece of intact hydrogel which was about 2 cm by about 2 cm. This piece of gel was then placed over the square hole in the collagen. In this case, the hydrogel protective covering extended over the square hole and onto the collagen. A PEG-3350-SPA hydrogel, as prepared in Example 3, was then applied over the top of the hole, the hydrogel, and surrounding collagen. The hydrogel was then allowed to set up for about 5 minutes. After curing, the collagen layers were separated easily to yield a uniform hydrogel covering the hole. The collagen was placed into a pH 7.4 PBS solution to swell in a 37° C. oven over night. Upon removal from the solution, it was observed that the gel had puckered and the upper hydrogel layer was only connected to the top collagen layer from the edges of the underlying hydrogel outwards to the edge of the collagen sheet.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of bandaging, covering, or bridging a defect, a wound, or a void of the tissue of a patient, comprising the steps of:
covering the defect, wound, or void with a first material; and
covering the first material with a second material;
wherein the area covered by the first material is greater than or equal to the area of the defect, wound, or void; the second material covers the area covered by the first material;
and the area covered by the second material is greater than the area covered by the first material.

2. The method of claim 1, wherein the defect, wound, or void is located in the dura.

3. The method of claim 1, wherein the defect, wound, or void is in the dura matter.

4. The method of claim 1, wherein the first material is brushed onto the defect, wound, or void.

5. The method of claim 1, wherein the first material is sprayed onto the defect, wound, or void.

6. The method of claim 1, wherein the first material is applied via a cannula onto the defect, wound, or void.

7. The method of claim 1, wherein the first material is biodegradable.

8. The method of claim 1, wherein the first material is substantially degraded, displaced, or diluted faster than the second material.

9. The method of claim 1, wherein the first material is substantially degraded, displaced, or diluted in about 30 minutes.

10. The method of claim 1, wherein the first material is substantially degraded, displaced, or diluted in about 2 hours.

11. The method of claim 1, wherein the first material is substantially degraded, displaced, or diluted in about 12 hours.

12. The method of claim 1, wherein the first material is substantially degraded, displaced, or diluted in about 24 hours.

13. The method of claim 1, wherein the first material is poly(lactic acid), poly(glycolic acid), or a copolymer thereof.

14. The method of claim 1, wherein the first material comprises collagen, hyaluronic acid, albumin, cellulose, elastin, fibrin, fibronectin, gelatine, heparin, heparin sulfate, polylysine, poly(vinyl acetate), polyvinylpyrrolidone, poly(acrylic acid), poly(ethylene glycol), poly(propylene glycol)-poly(ethylene glycol) copolymer, trimethylene carbonate, or a polypeptide comprising the tripeptide Arg-Gly-Asp.

15. The method of claim 1, wherein the first material is a hydrogel.

16. The method of claim 1, wherein the first material is a pre-formed hydrogel.

17. The method of claim 1, wherein the first material is a polyalkyleneimine-containing hydrogel.

18. The method of claim 1, wherein the first material is a hydrogel; and the hydrogel has pores in the range of about 1 micron to about 100 microns in diameter.

19. The method of claim 1, wherein the first material comprises a medicament, a colorant, a flavoring, a scent, a fibrous additive, a thickener or a plasticizer.

20. The method of claim 1, wherein the second material is brushed onto the defect, wound, or void.

21. The method of claim 1, wherein the second material is sprayed onto the defect, wound, or void.

22. The method of claim 1, wherein the second material is applied via a cannula onto the defect, wound, or void.

23. The method of claim 1, wherein the second material is biodegradable.

24. The method of claim 1, wherein the second material is a hydrogel.

25. The method of claim 1, wherein the second material is a pre-formed hydrogel.

26. The method of claim 1, wherein the second material is a polyalkyleneimine-containing hydrogel.

27. The method of claim 1, wherein said the second material is a hydrogel; and said hydrogel has pores in the range of about 1 micron to about 100 microns in diameter.

28. The method of claim 1, wherein the second material comprises a medicament, a colorant, a flavoring, a scent, a fibrous additive, a thickener or a plasticizer.

29. The method of claim 1, further comprising the step of placing a piece of sterile material around the defect, wound, or void, so that only the defect, wound, or void is exposed, prior to placing the first material over the defect, wound, or void.

30. The method of claim 1, further comprising the step of applying a dissolvable polymer or inorganic salt to the wound, void, or tissue of a patient.

31. The method of claim 1, further comprising the step of applying a mesh to the wound, void, or tissue of a patient.

32. The method of claim 1, further comprising the step of applying a mesh to the wound, void, or tissue of a patient; wherein the mesh comprises methylmethacrylate, mersilene, silicone, Teflon®, Dacron®, polyethylene, polyester, titanium-Dacron®, hydroxylapatite, or combinations thereof.

33. The method of claim 1, further comprising the step of applying a mesh to the wound, void, or tissue of a patient; wherein the mesh comprises polypropylene or polyester.

34. The method of claim 1, further comprising the step of applying a mesh to the wound, void, or tissue of a patient; wherein the mesh comprises a biodegradable polymer.

35. The method of claim 1, further comprising the step of applying a mesh to the wound, void, or tissue of a patient; wherein the mesh comprises poly(glycolic acid), poly(lactic acid), or copolymers thereof.

36. The method of claim 1, wherein the first material comprises a mesh.

37. The method of claim 1, wherein the first material comprises a mesh; and the mesh comprises methylmethacrylate, mersilene, silicone, Teflon®, Dacron®, polyethylene, polyester, titanium-Dacron®, hydroxylapatite, or combinations thereof.

38. The method of claim 1, wherein the first material comprises a mesh; and the mesh comprises polypropylene or polyester.

39. The method of claim 1, wherein the first material comprises a mesh; and the mesh comprises a biodegradable polymer.

40. The method of claim 1, wherein the first material comprises a mesh; and the mesh comprises poly(glycolic acid), poly(lactic acid), or copolymers thereof.

41. The method of claim 1, wherein the second material comprises a mesh.

42. The method of claim 1, wherein the second material comprises a mesh; and the mesh comprises methylmethacrylate, mersilene, silicone, Teflon®, Dacron®, polyethylene, polyester, titanium-Dacron®, hydroxylapatite, or combinations thereof.

43. The method of claim 1, wherein the second material comprises a mesh; and the mesh comprises polypropylene or polyester.

44. The method of claim 1, wherein the second material comprises a mesh; and the mesh comprises a biodegradable polymer.

45. The method of claim 1, wherein the second material comprises a mesh; and the mesh comprises poly(glycolic acid), poly(lactic acid), or copolymers thereof.

46. The method of claim 1, wherein the first material is a hydrogel; and the step of covering the defect, wound, or void with a first material comprises the steps of:
applying a first composition to the defect, wound or void; and
applying a second composition to the defect, wound or void,
wherein, after a first amount of time, application of the first composition and application of the second composition results in the formation of the first material.

47. The method of claim 1, wherein the first material is a hydrogel; and the step of covering the defect, wound, or void with a first material comprises the step of:
applying a pre-hydrogel mixture to the defect, wound, or void;
wherein said pre-hydrogel mixture comprises a first composition and a second composition; and, after a first amount of time, the pre-hydrogel mixture gels, thereby forming the first material.

48. The method of claim 1, further comprising the steps of:
exposing the first material, the second material, or both the first material and the second material, to a third composition, thereby forming a photo-polymerizable material; and
exposing the photo-polymerizable material to ultraviolet or visible light, thereby polymerizing the photo-polymerizable material;
wherein said third composition comprises a compound of formula V: 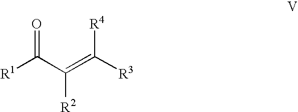
wherein, independently for each occurrence,
R¹ is a halogen, 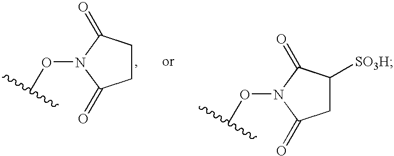
R² hydrogen, alkyl, aryl, or aralkyl;
R³ hydrogen, alkyl, aryl, or aralkyl; and
R⁴ is hydrogen, alkyl, aryl, or aralkyl.

49. The method of claim 1, wherein the step of covering the defect, wound, or void with a first material comprises the steps of:
applying a composition to the defect, wound or void; and
treating the composition with ultraviolet light or visible light sufficient to polymerize said the composition, thereby forming the first material.

50. The method of claim 1, wherein the step of covering the first material with a second material comprises the steps of:
applying a composition to cover the first material; and
treating the composition with ultraviolet light or visible light sufficient to polymerize said the composition, thereby forming the second material.
